SEQUENCE LISTING (7 sequences)

SEQ ID NO: 1 - protein, 1401 aa, Homo sapiens
MAPQMYEFHLPLSPEELLKSGGVNQYVVQEVLSIKHLPPQLRAFQAAFRAQGPLAMLQHFDTIYSILHHFRSIDPGLKEDTLQFLIKVVSRHSQELPAILDDTTLSGSDRNAHLNALKMNCYALIRLLESFETMASQTNLVDLDLGGKGKKARTKAAHGFDWEEERQPILQLLTQLLQLDIRHLWNHSIIEEEFVSLVTGCCYRLLENPTINHQKNRPTREAITHLLGVALTRYNHMLSATVKIIQMLQHFEHLAPVLVAAVSLWATDYGMKSIVGEIVREIGQKCPQELSRDPSGTKGFAAFLTELAERVPAILMSSMCILLDHLDGENYMMRNAVLAAMAEMVLQVLSGDQLEAAARDTRDQFLDTLQAHGHDVNSFVRSRVLQLFTRIVQQKALPLTRFQAVVALAVGRLADKSVLVCKNAIQLLASFLANNPFSCKLSDADLAGPLQKETQKLQEMRAQRRTAAASAVLDPEEEWEAMLPELKSTLQQLLQLPQGEEEIPEQIANTETTEDVKGRIYQLLAKASYKKAIILTREATGHFQESEPFSHIDPEESEETRLLNILGLIFKGPAASTQEKNPRESTGNMVTGQTVCKNKPNMSDPEESRGNDELVKQEMLVQYLQDAYSFSRKITEAIGIISKMMYENTTTVVQEVIEFFVMVFQFGVPQALFGVRRMLPLIWSKEPGVREAVLNAYRQLYLNPKGDSARAKAQALIQNLSLLLVDASVGTIQCLEEILCEFVQKDELKPAVTQLLWERATEKVACCPLERCSSVMLLGMMARGKPEIVGSNLDTLVSIGLDEKFPQDYRLAQQVCHAIANISDRRKPSLGKRHPPFRLPQEHRLFERLRETVTKGFVHPDPLWIPFKEVAVTLIYQLAEGPEVICAQILQGCAKQALEKLEEKRTSQEDPKESPAMLPTFLLMNLLSLAGDVALQQLVHLEQAVSGELCRRRVLREEQEHKTKDPKEKNTSSETTMEEELGLVGATADDTEAELIRGICEMELLDGKQTLAAFVPLLLKVCNNPGLYSNPDLSAAASLALGKFCMISATFCDSQLRLLFTMLEKSPLPIVRSNLMVATGDLAIRFPNLVDPWTPHLYARLRDPAQQVRKTAGLVMTHLILKDMVKVKGQVSEMAVLLIDPEPQIAALAKNFFNELSHKGNAIYNLLPDIISRLSDPELGVEEEPFHTIMKQLLSYITKDKQTESLVEKLCQRFRTSRTERQQRDLAYCVSQLPLTERGLRKMLDNFDCFGDKLSDESIFSAFLSVVGKLRRGAKPEGKAIIDEFEQKLRACHTRGLDGIKELEIGQAGSQRAPSAKKPSTGSRYQPLASTASDNDFVTPEPRRTTRRHPNTQQRASKKKPKVVFSSDESSEEDLSAEMTEDETPKKTTPILRASARRHRS

SEQ ID NO: 2 - protein, 377 aa, Homo sapiens
MCDEDETTALVCDNGSGLVKAGFAGDDAPRAVFPSIVGRPRHQGVMVGMGQKDSYVGDEAQSKRGILTLKYPIEHGIITNWDDMEKIWHHTFYNELRVAPEEHPTLLTEAPLNPKANREKMTQIMFETFNVPAMYVAIQAVLSLYASGRTTGIVLDSGDGVTHNVPIYEGYALPHAIMRLDLAGRDLTDYLMKILTERGYSFVTTAEREIVRDIKEKLCYVALDFENEMATAASSSSLEKSYELPDGQVITIGNERFRCPETLFQPSFIGMESAGIHETTYNSIMKCDIDIRKDLYANNVMSGGTTMYPGIADRMQKEITALAPSTMKIKIIAPPERKYSVWIGGSILASLSTFQQMWITKQEYDEAGPSIVHRKCF

SEQ ID NO: 3 - protein, 467 aa, Homo sapiens
MSSILPFTPPVVKRLLGWKKSAGGSGGAGGGEQNGQEEKWCEKAVKSLVKKLKKTGRLDELEKAITTQNCNTKCVTIPSTCSEIWGLSTPNTIDQWDTTGLYSFSEQTRSLDGRLQVSHRKGLPHVIYCRLWRWPDLHSHHELKAIENCEYAFNLKKDEVCVNPYHYQRVETPVLPPVLVPRHTEILTELPPLDDYTHSIPENTNFPAGIEPQSNYIPETPPPGYISEDGETSDQQLNQSMDTGSPAELSPTTLSPVNHSLDLQPVTYSEPAFWCSIAYYELNQRVGETFHASQPSLTVDGFTDPSNSERFCLGLLSNVNRNATVEMTRRHIGRGVRLYYIGGEVFAECLSDSAIFVQSPNCNQRYGWHPATVCKIPPGCNLKIFNNQEFAALLAQSVNQGFEAVYQLTRMCTIRMSFVKGWGAEYRRQTVTSTPCWIELHLNGPLQWLDKVLTQMGSPSVRCSSMS

SEQ ID NO: 4 - protein, 795 aa, Homo sapiens
MDTKLNMLNEKVDQLLHFQEDVTEKLQSMCRDMGHLERGLHRLEASRAPGPGGADGVPHIDTQAGWPEVLELVRAMQQDAAQHGARLEALFRMVAAVDRAIALVGATFQKSKVADFLMQGRVPWRRGSPGDSPEENKERVEEEGGKPKHVLSTSGVQSDAREPGEESQKADVLEGTAERLPPIRASGLGADPAQAVVSPGQGDGVPGPAQAFPGHLPLPTKVEAKAPETPSENLRTGLELAPAPGRVNVVSPSLEVAPGAGQGASSSRPDPEPLEEGTRLTPGPGPQCPGPPGLPAQARATHSGGETPPRISIHIQEMDTPGEMLMTGRGSLGPTLTTEAPAAAQPGKQGPPGTGRCLQAPGTEPGEQTPEGARELSPLQESSSPGGVKAEEEQRAGAEPGTRPSLARSDDNDHEVGALGLQQGKSPGAGNPEPEQDCAARAPVRAEAVRRMPPGAEAGSVVLDDSPAPPAPFEHRVVSVKETSISAGYEVCQHEVLGGGRFGQVHRCTEKSTGLPLAAKIIKVKSAKDREDVKNEINIMNQLSHVNLIQLYDAFESKHSCTLVMEYVDGGELFDRITDEKYHLTELDVVLFTRQICEGVHYLHQHYILHLDLKPENILCVNQTGHQIKIIDFGLARRYKPREKLKVNFGTPEFLAPEVVNYEFVSFPTDMWSVGVITYMLLSGLSPFLGETDAETMNFIVNCSWDFDADTFEGLSEEAKDFVSRLLVKEKSCRMSATQCLKHEWLNNLPAKASRSKTRLKSQLLLQKYIAQRKWKKHFYVVTAANRLRKFPTSP

SEQ ID NO: 5 - protein, 2477 aa, Homo sapiens
MLRGPGPGLLLLAVQCLGTAVPSTGASKSKRQAQQMVQPQSPVAVSQSKPGCYDNGKHYQINQQWERTYLGNALVCTCYGGSRGFNCESKPEAEETCFDKYTGNTYRVGDTYERPKDSMIWDCTCIGAGRGRISCTIANRCHEGGQSYKIGDTWRRPHETGGYMLECVCLGNGKGEWTCKPIAEKCFDHAAGTSYVVGETWEKPYQGWMMVDCTCLGEGSGRITCTSRNRCNDQDTRTSYRIGDTWSKKDNRGNLLQCICTGNGRGEWKCERHTSVQTTSSGSGPFTDVRAAVYQPQPHPQPPPYGHCVTDSGVVYSVGMQWLKTQGNKQMLCTCLGNGVSCQETAVTQTYGGNSNGEPCVLPFTYNGRTFYSCTTEGRQDGHLWCSTTSNYEQDQKYSFCTDHTVLVQTRGGNSNGALCHFPFLYNNHNYTDCTSEGRRDNMKWCGTTQNYDADQKFGFCPMAAHEEICTTNEGVMYRIGDQWDKQHDMGHMMRCTCVGNGRGEWTCIAYSQLRDQCIVDDITYNVNDTFHKRHEEGHMLNCTCFGQGRGRWKCDPVDQCQDSETGTFYQIGDSWEKYVHGVRYQCYCYGRGIGEWHCQPLQTYPSSSGPVEVFITETPSQPNSHPIQWNAPQPSHISKYILRWRPKNSVGRWKEATIPGHLNSYTIKGLKPGVVYEGQLISIQQYGHQEVTRFDFTTTSTSTPVTSNTVTGETTPFSPLVATSESVTEITASSFVVSWVSASDTVSGFRVEYELSEEGDEPQYLDLPSTATSVNIPDLLPGRKYIVNVYQISEDGEQSLILSTSQTTAPDAPPDTTVDQVDDTSIVVRWSRPQAPITGYRIVYSPSVEGSSTELNLPETANSVTLSDLQPGVQYNITIYAVEENQESTPVVIQQETTGTPRSDTVPSPRDLQFVEVTDVKVTIMWTPPESAVTGYRVDVIPVNLPGEHGQRLPISRNTFAEVTGLSPGVTYYFKVFAVSHGRESKPLTAQQTTKLDAPTNLQFVNETDSTVLVRWTPPRAQITGYRLTVGLTRRGQPRQYNVGPSVSKYPLRNLQPASEYTVSLVAIKGNQESPKATGVFTTLQPGSSIPPYNTEVTETTIVITWTPAPRIGFKLGVRPSQGGEAPREVTSDSGSIVVSGLTPGVEYVYTIQVLRDGQERDAPIVNKVVTPLSPPTNLHLEANPDTGVLTVSWERSTTPDITGYRITTTPTNGQQGNSLEEVVHADQSSCTFDNLSPGLEYNVSVYTVKDDKESVPISDTIIPEVPQLTDLSFVDITDSSIGLRWTPLNSSTIIGYRITVVAAGEGIPIFEDFVDSSVGYYTVTGLEPGIDYDISVITLINGGESAPTTLTQQTAVPPPTDLRFTNIGPDTMRVTWAPPPSIDLTNFLVRYSPVKNEEDVAELSISPSDNAVVLTNLLPGTEYVVSVSSVYEQHESTPLRGRQKTGLDSPTGIDFSDITANSFTVHWIAPRATITGYRIRHHPEHFSGRPREDRVPHSRNSITLTNLTPGTEYVVSIVALNGREESPLLIGQQSTVSDVPRDLEVVAATPTSLLISWDAPAVTVRYYRITYGETGGNSPVQEFTVPGSKSTATISGLKPGVDYTITVYAVTGRGDSPASSKPISINYRTEIDKPSQMQVTDVQDNSISVKWLPSSSPVTGYRVTTTPKNGPGPTKTKTAGPDQTEMTIEGLQPTVEYVVSVYAQNPSGESQPLVQTAVTNIDRPKGLAFTDVDVDSIKIAWESPQGQVSRYRVTYSSPEDGIHELFPAPDGEEDTAELQGLRPGSEYTVSVVALHDDMESQPLIGTQSTAIPAPTDLKFTQVTPTSLSAQWTPPNVQLTGYRVRVTPKEKTGPMKEINLAPDSSSVVVSGLMVATKYEVSVYALKDTLTSRPAQGVVTTLENVSPPRRARVTDATETTITISWRTKTETITGFQVDAVPANGQTPIQRTIKPDVRSYTITGLQPGTDYKIYLYTLNDNARSSPVVIDASTAIDAPSNLRFLATTPNSLLVSWQPPRARITGYIIKYEKPGSPPREVVPRPRPGVTEATITGLEPGTEYTIYVIALKNNQKSEPLIGRKKTDELPQLVTLPHPNLHGPEILDVPSTVQKTPFVTHPGYDTGNGIQLPGTSGQQPSVGQQMIFEEHGFRRTTPPTTATPIRHRPRPYPPNVGEEIQIGHIPREDVDYHLYPHGPGLNPNASTGQEALSQTTISWAPFQDTSEYIISCHPVGTDEEPLQFRVPGTSTSATLTGLTRGATYNVIVEALKDQQRHKVREEVVTVGNSVNEGLNQPTDDSCFDPYTVSHYAVGDEWERMSESGFKLLCQCLGFGSGHFRCDSSRWCHDNGVNYKIGEKWDRQGENGQMMSCTCLGNGKGEFKCDPHEATCYDDGKTYHVGEQWQKEYLGAICSCTCFGGQRGWRCDNCRRPGGEPSPEGTTGQSYNQYSQRYHQRTNTNVNCPIECFMPLDVQADREDSRE

SEQ ID NO: 6 - protein, 1464 aa, Homo sapiens
MFSFVDLRLLLLLAATALLTHGQEEGQVEGQDEDIPPITCVQNGLRYHDRDVWKPEPCRICVCDNGKVLCDDVICDETKNCPGAEVPEGECCPVCPDGSESPTDQETTGVEGPKGDTGPRGPRGPAGPPGRDGIPGQPGLPGPPGPPGPPGPPGLGGNFAPQLSYGYDEKSTGGISVPGPMGPSGPRGLPGPPGAPGPQGFQGPPGEPGEPGASGPMGPRGPPGPPGKNGDDGEAGKPGRPGERGPPGPQGARGLPGTAGLPGMKGHRGFSGLDGAKGDAGPAGPKGEPGSPGENGAPGQMGPRGLPGERGRPGAPGPAGARGNDGATGAAGPPGPTGPAGPPGFPGAVGAKGEAGPQGPRGSEGPQGVRGEPGPPGPAGAAGPAGNPGADGQPGAKGANGAPGIAGAPGFPGARGPSGPQGPGGPPGPKGNSGEPGAPGSKGDTGAKGEPGPVGVQGPPGPAGEEGKRGARGEPGPTGLPGPPGERGGPGSRGFPGADGVAGPKGPAGERGSPGPAGPKGSPGEAGRPGEAGLPGAKGLTGSPGSPGPDGKTGPPGPAGQDGRPGPPGPPGARGQAGVMGFPGPKGAAGEPGKAGERGVPGPPGAVGPAGKDGEAGAQGPPGPAGPAGERGEQGPAGSPGFQGLPGPAGPPGEAGKPGEQGVPGDLGAPGPSGARGERGFPGERGVQGPPGPAGPRGANGAPGNDGAKGDAGAPGAPGSQGAPGLQGMPGERGAAGLPGPKGDRGDAGPKGADGSPGKDGVRGLTGPIGPPGPAGAPGDKGESGPSGPAGPTGARGAPGDRGEPGPPGPAGFAGPPGADGQPGAKGEPGDAGAKGDAGPPGPAGPAGPPGPIGNVGAPGAKGARGSAGPPGATGFPGAAGRVGPPGPSGNAGPPGPPGPAGKEGGKGPRGETGPAGRPGEVGPPGPPGPAGEKGSPGADGPAGAPGTPGPQGIAGQRGVVGLPGQRGERGFPGLPGPSGEPGKQGPSGASGERGPPGPMGPPGLAGPPGESGREGAPGAEGSPGRDGSPGAKGDRGETGPAGPPGAPGAPGAPGPVGPAGKSGDRGETGPAGPAGPVGPVGARGPAGPQGPRGDKGETGEQGDRGIKGHRGFSGLQGPPGPPGSPGEQGPSGASGPAGPRGPPGSAGAPGKDGLNGLPGPIGPPGPRGRTGDAGPVGPPGPPGPPGPPGPPSAGFDFSFLPQPPQEKAHDGGRYYRADDANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKDKRHVWFGESMTDGFQFEYGGQGSDPADVAIQLTFLRLMSTEASQNITYHCKNSVAYMDQQTGNLKKALLLQGSNEIEIRAEGNSRFTYSVTVDGCTSHTGAWGKTVIEYKTTKTSRLPIIDVAPLDVGAPDQEFGFDVGPVCFL

SEQ ID NO: 7 - protein, 390 aa, Homo sapiens
MPPSGLRLLPLLLPLLWLLVLTPGRPAAGLSTCKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSRDNTLQVDINGFTTGRRGDLATIHGMNRPFLLLMATPLERAQHLQSSRHRRALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

(19) United States
(12) Patent Application Publication
(10) Pub. No.: US 2024/0402153 A1
(43) Pub. Date: Dec. 5, 2024

(54) SYSTEM, DISEASE MODEL AND METHODS OF USING THE SAME

(71) Applicant: THE ADMINISTRATORS OF THE TULANE EDUCATIONAL FUND, New Orleans, LA (US)

(72) Inventors: Mark Justin MONDRINOS, New Orleans, LA (US); Elisabet OLSEN, Memphis, TN (US)

(21) Appl. No.: 18/699,514

(22) PCT Filed: Oct. 7, 2022
(86) PCT No.: PCT/US22/77820

§ 371 Date: Apr. 8, 2024

Related U.S. Application Data
(60) Provisional application No. 63/253,441, filed on Oct. 7, 2021.

(51) Int. Cl. G01N 33/50; C12N 5/071; C12N 5/09

(52) U.S. Cl. CPC G01N 33/5011; C12N 5/0691; C12N 5/0693; C12N 5/0697; G01N 33/5082; C12N 2513/00; C12N 2533/40

(57) ABSTRACT

Disclosed are designs that enable rapid production of fully vascularized milliscale explant tissues that will enable preservation and expansion of this precious clinical resource and simultaneous investigation of potential biological underpinnings of diseases. Models include triple negative breast cancer or aggressive lung cancer and quiescent lung interstitium integrated in a robust, easily adaptable millifluidic device. Devices will allow any laboratory that use cultures and/or organoids to construct complex multi-tissue/organ systems. Implementation of these devices can model multiple organ pathologies induced by tumor-derived factors.

### SEQUENCE LISTING

The contents of the electronic sequence listing submitted herewith as file 37926.0007P1FINAL.xml; Size: 14,385 bytes; and Date of Creation: Oct. 7, 2022, is herein incorporated by reference in its entirety.

### TECHNOLOGY FIELD

The present disclosure relates to vascularized milliscale organ chip systems for applications in biomedically relevant fields. The disclosure also relates to the methods of making and using the system embodiments for functional bioassays.

### BACKGROUND

Organ chip systems have numerous biomedically relevant applications and have seen an increase in use in biomedically relevant fields, such as disease modeling, tissue engineering, therapeutics research, etc. Organ chip systems are a promising alternative to study disease and disease progression utilizing human-sourced materials and without need for traditional animal models. Organ chip systems have traditionally focused on single-organ systems but are being expanded to include multiple organ systems to improve disease pathology and its progression throughout the body.

There are several key impediments that limit the potential impact of microphysiological systems (MPS) in basic, preclinical, and translational research. Key among these are: i) difficulty implementing MPS platforms that results in high experimental failure rates; ii) incompatibility of miniaturized volumes with standard methods for biochemical analysis of tissue homogenates widely used in biomedical research labs.

MPS technologies that work reliably in the hands of non-expert users and fit their experimental workflows are needed to accelerate biological discovery. Our objectives were to address these needs by: i) establishing a digital manufacturing (DM) workflow that enables rapid prototyping and organ chip design iteration; ii) designing milliscale organ chip systems that accommodate larger tissue volumes; iii) using failure testing to establish reduction to practice of organ chip systems.

Microphysiological systems (MPS) offer the opportunity to model complex pathophysiology, including multiple organ pathologies induced by cancers, in an all-human in vitro system. The impact of MPS in preclinical research will be determined in part by the ability of non-expert labs to adopt the technology.

Triple negative breast cancer (TNBC) is a lethal disease that features a prominent health disparity amongst African American (AA) women. Low survival rates in lung cancer can be attributed to high rates of metastasis. Increasing evidence reveals premetastatic niches (PMN) promote colonization in distant tissues and organs. Tumor-derived factors (TDF) such as cytokines, growth factors, and exosomes, facilitate PMN cultivation via mechanisms including activation of stromal cells and acute extracellular matrix (ECM) deposition.

### SUMMARY

Our objective is to develop a fully vascularized microphysiological model of TNBC using patient biopsies. We manufactured a milliscale membrane free organ chip (MFOC) that enables reproducible loading and rapid formation of perfusable vasculature spanning tissues greater than 1 mm in the shortest dimension. In parallel, we developed methods of TNBC biopsy processing that yield injectable explants for membrane free organ chip seeding to create the integrated microphysiological model.

We engineered a microphysiological model of PMN cultivation induced by lung cancer tumor-derived factors (TDF) in a region of normal lung tissue. The core components of the integrated MPS are: i) a 3D model of aggressive non-small cell lung cancer (NSCLC); ii) a 3D model of quiescent lung interstitium in which PMN cultivation is induced within a tissue exhibiting a physiological resting state; iii) a multilayer fluidic device containing two interconnected and individually addressable tissue/organ modules to facilitate real-time TDF transport. In these studies, we designed, tested, and validated a prototype 4-organ chip that enables user-friendly injection of hydrogels containing mixtures of cells and/or spheroids/organoids.

In one aspect, the disclosure relates to 1. A composition comprising: (a) a solid substrate comprising a first compartment and a second compartment; the first and second compartments in fluid communication with each other; (b) a plurality of cancer cells; (c) a plurality of endothelial cells; and wherein the cancer cells and the endothelial cells are in separate compartments in fluid communication; and wherein the composition is membrane-free.

In some embodiments, the composition is not more than 2 millimeters in length.

In some embodiments, the endothelial cells are structurally organized in an in vitro blood vessel structure fluidly connecting the cancer cells with a group of non-cancer cells.

In some embodiments, a layer of hydrogel is positioned at the interface of the first and second compartments.

In some embodiments, the hydrogel comprises one or a combination of: Collagen I, Collagen III and Fibronectin.

In some embodiments, the hydrogel has a density above about 75 mg/per mL of hydrogel volume.

In some embodiments, the hydrogel has a density above about 100 mg/per mL of hydrogel volume.

In some embodiments, the cancer cells are from a biopsy.

In some embodiments, the biopsy sample is triple negative breast cancer.

In some embodiments, the composition further comprises cells from a single organ.

In some embodiments, the composition further comprises cells from at least two organs in two distinct compartments.

In some embodiments, the cancer cells are carcinoma cells from a human lung, human breast or human colon.

In some embodiments the cancer cells are organized in a first layer of cells; wherein the endothelial cells are organized in a second layer of cells, and dense hydrogel is positioned at the interface between the first and second layer of cells.

In some embodiments, the cancer cells are organized in a spheroid.

In some embodiments, the composition further comprises non-cancer cells in a compartment at a point distal from the other compartment.

In some embodiments, the composition comprises a first, second, third and fourth compartment, each compartment separated by a hydrogel layer and each compartment interface free of a membrane and in fluid communication with each other.

In some embodiments, one or a plurality of compartments has a volume of from about 30 to about 50 millimeter cubed.

In some embodiments, the composition further comprises stromal cells in a layer of hydrogel.

In some embodiments, the stromal cells are human fibroblasts.

In some embodiments, the human fibroblasts are primary human fibroblasts.

In some embodiments, the stromal cells are at density from about 500,000 cells per milliliter of volume of the compartment to about 1,500,000 cells per milliliter of volume of the compartment.

The present disclosure also relates to a system comprising the composition of any of claims 1 through 21 and a tissue culture media.

In some embodiments, the non-cancer cells are in contact within a tissue culture media.

In some embodiments, the system further comprises a heating element and an outlet positioned proximate to the composition, wherein the outlet is in fluid communication with an oxygen and nitrogen source.

The present disclosure is also related to a method of assaying the toxicity or therapeutic effectiveness of an agent on a cancer cell comprising: (a) contacting the composition of any of claims 1 through 21 with an agent.

In some embodiments, the method further comprises a step of (b) monitoring the cells for morphologic changes or changes of expression profile of cells after step (a).

In some embodiments, the agent is chosen from one or a combination of: an environmental agent, a small molecule therapeutic, a biologic immunotherapy, or a modified T cell.

In some embodiments, the agent is a biologic immunotherapy that is an antibody or antibody fragment thereof.

In some embodiments, the agent is a modified cell that is a CAR-T cell.

The present disclosure is also related to a method of screening an agent or library of agents for efficacy as a cancer therapeutic for pre-metastatic effector comprising: (a) contacting the composition of any of claims 1 through 21 with an agent.

The present disclosure is also related to a method of manufacturing a cell culture comprising: (a) seeding a plurality of cancer cells; and (b) seeding a plurality of endothelial cells for a time period sufficient for the endothelial cells to fluidically connect the first and second compartment of the composition of any of claims 1 through 21.

In some embodiments, the time period is no less than about seven days.

In some embodiments, the composition further comprises a layer of stromal cells and extracellular matrix protein or proteins positioned at the interface of the first and second compartment, and wherein the time period is sufficient to deposit extracellular protein density around the plurality of cancer cells equivalent to from about 6 KPa to about 10 KPa.

In some embodiments, the method further comprises allowing the cells to divide until there are from about 400,000 cells per milliliter to about 1,000,000 of stromal cells per milliliter of volume in a vessel.

In some embodiments, the composition is capable of fluid exchange through diffusion between the first or second compartments.

### BRIEF DESCRIPTION OF THE DRAWINGS

The following drawings form part of the present specification and are included to further demonstrate certain aspects of the present invention. The invention may be better understood by reference to one or more of these drawings in combination with the description of specific embodiments presented herein.

FIG. 1. Design, testing and reduction to practice of membrane free organ chip (MFOC) as a 100% success rate platform for rapid production of vascularized milliscale organ chips. A. MFOC design render showing upper and lower tissue guide layers of our double guide MFOC; cross-sectional diagram showing the key dimensions optimized in our design convergence; h (tissue wall height), H (tissue height), W (tissue width), G (guide width). B. Examples of MFOC loading success and failure (blowout) in our reduction to practice study. C. Anchored boundary of vascularized tissue on the guide struction of a single lane MFOC, lecting labeling of endothelial cells (red, UEA-1), 5 days of culture, inset: Tissue wall geometry visualized by loading with FITC-dextran. 3D projection in cross-sectional view.

FIG. 2. 4-organ device. A. A combination of food dye colors to visualize the compartmentalization of our devices, which feature a dedicated fluidic connection for each organ module and a shared fluidic connection for factor exchange between organ modules (inside channel of octagon with dark blue dye). B. Tissue vascularization visualized by UEA-1 staining (red). Supporting fibroblasts are seen in green.

FIG. 3. Milliscale membrane free organ chip (MFOC) devices enable rapid formation of large fully vascularized TNBC explant tissues. A. MFOC device in culture and cross section rendering of the multi-guide design enabling high liquid pinning success rates of volumes up to 1.5×2×12 mm. B. TNBC injectable explant tissue; Ki67, 3 days in MFOC (dense, viable TNBC tissues with large fractions of Ki67-positive proliferating cells in short-term MFOC culture). C. 3D projection of tissue wall geometry in cross section. D. Top down view of fully anastomosed internal vasculature in MFOC at the tissue boundary region shown in Panel C. E. Fully vascularized prototype using patient-derived TNBC spheroids with perfusable vasculature spanning 1.5 mm. Refer to Panel A for reference on guide locations. 5 days. F. FITC-dextran perfusion in the device shown in Panel E. D-F show tissue wall endothelialization and complete vascular network anastomosis.

FIG. 4. Microphysiological model of lung cancer premetastatic niche (PMN) cultivation. A. Digital photo of device, tissue chambers in red (medium gray), compartment media reservoirs in yellow (light gray), connecting channel for tumor-derived factors (TDF) transport in blue (darkest gray) (500 μm wide) (a multilayer fluidic device containing two interconnected and individually addressable tissue/organ modules to facilitate real-time TDF transport with a tumor microenvironment on the left and a normal lung environment on the right). B-G. Features of aggressive cancers after 7 days of culture. B. TME compartment, 7 days. Actin and fibronectin staining; z-stack=300 μm. C. TME compartment, 7 days; SMA and YAP staining; z-stack=300 μm. D. Fibronectin fiber alignment ***—P<0.001; t-test. E. PMN cultivation in lung interstitium; Actin and fibronectin staining; z-stack=300 μm. G. Total fibronectin length and matric lacunarity. ***—P<0.0001; t-test.

FIG. 5. Vascularizing Milliscale Tissues—vascularization of a double guide MFOC by 5 days in culture. Internal vasculature is perfusable upon seeding of the side channels and allowing for anastomosis (not shown here). A. Device with EGM loaded (static culture). B. Actin and UEA-1 staining. C. Vascular networks are fully formed by Day 7 in culture, as evidenced by the quantitative reduction of the number of non-participating, i.e. single, endothelial cells. DAPI (nuclie, blue), Actin (cytoskeleton), UEA-1 lectin (endothelial cells). D. Network assembly was quantified using custom MATLAB scripts that calculate the number of single endothelial cells not assembled into the contiguous network.

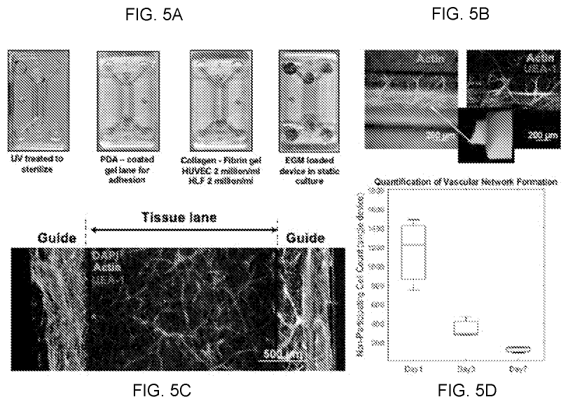

FIG. 6 shows engineered TNBC-derivatives processing workflow.

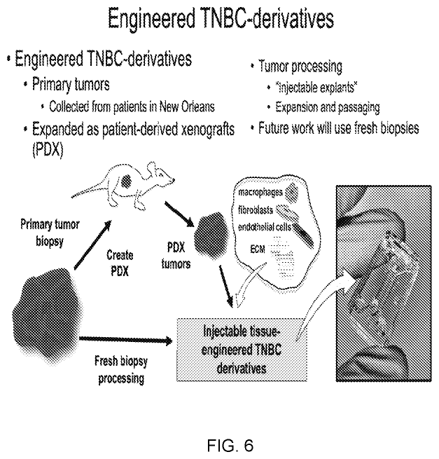

FIG. 7 shows a vascularized TNBC model.

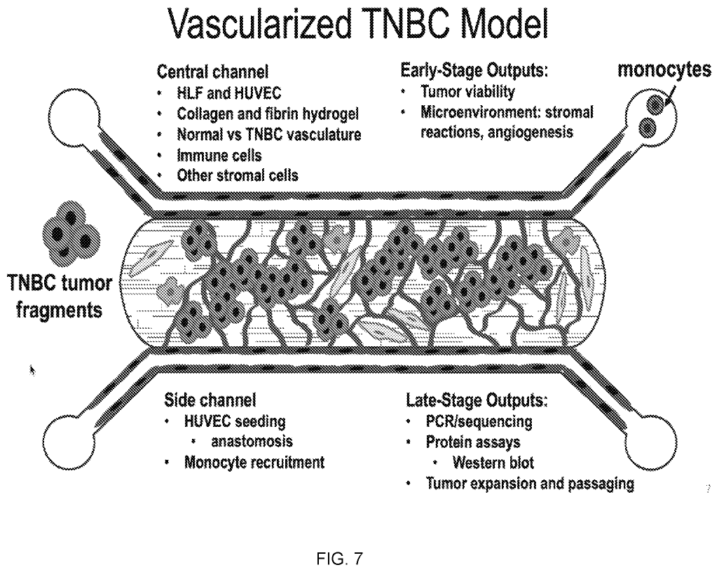

FIG. 8. Biopsy processing yields viable injectable explant. FIG. 8 shows that biopsy processing yields viable injectable explants.

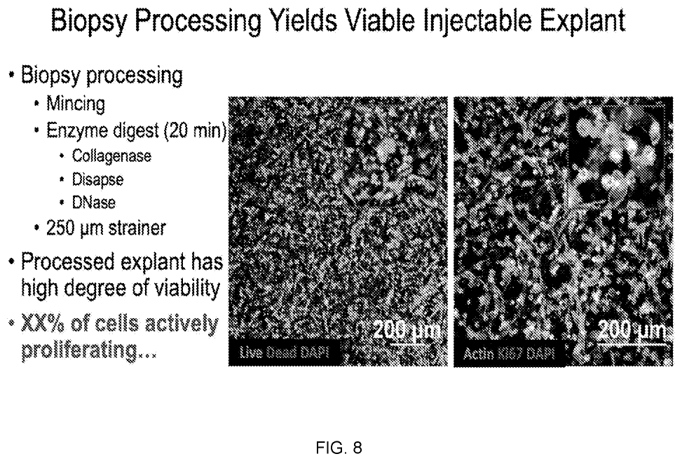

FIG. 9 shows results of rapid TNBC tissue vascularization.

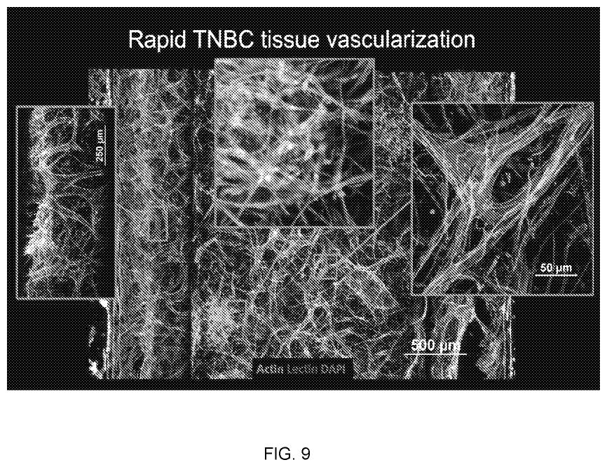

FIG. 10 shows normal v TNBC vasculature in organ chip system.

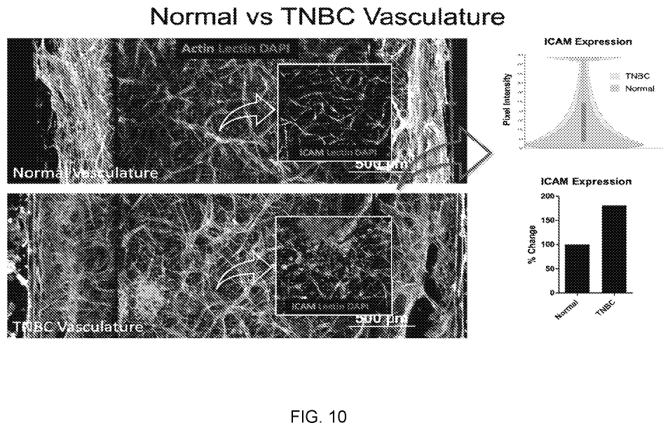

FIG. 11 shows design for multiple organ chip system.

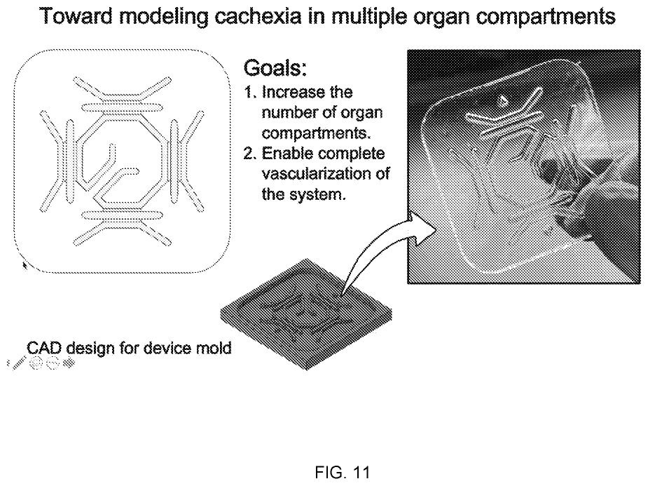

FIG. 12 shows cross-section of multiple organ chip system.

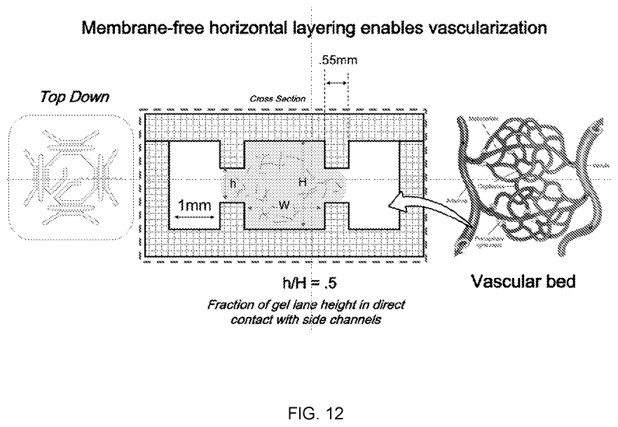

FIG. 13 shows vascularized stromal tissue.

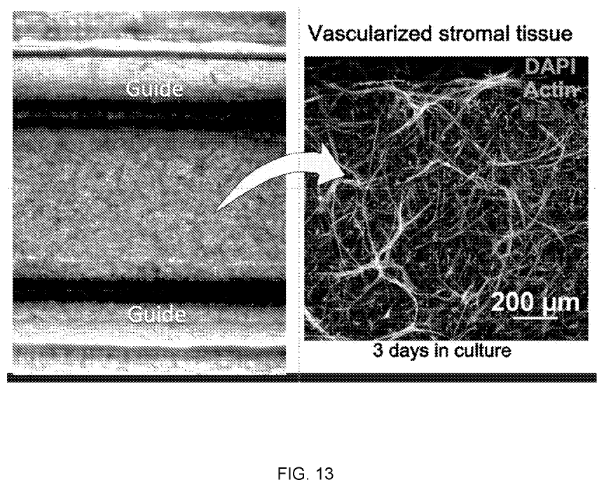

FIG. 14 shows vascularization of tissue between channels >1 mm after 5 days of culture.

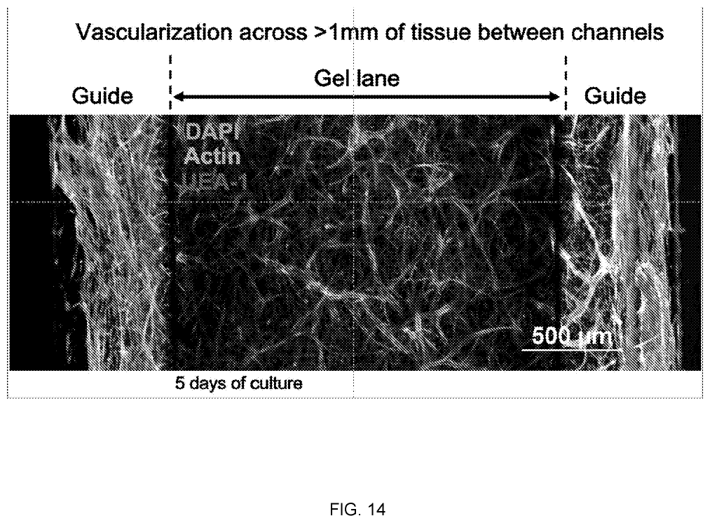

FIG. 15 shows cross-sectional view of vascularized MFOC and perfusability

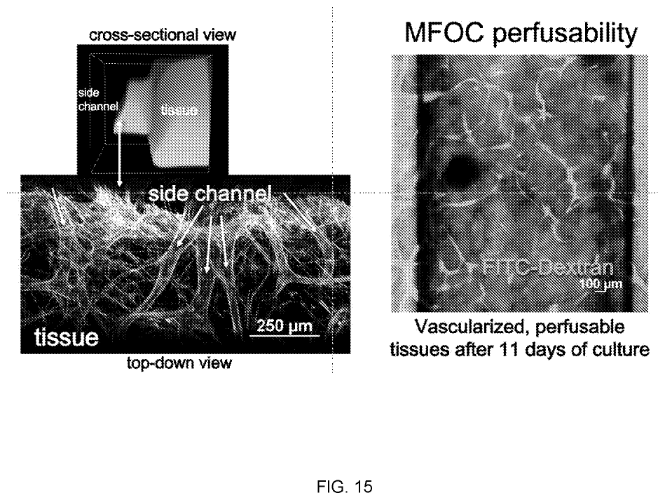

FIG. 16 shows multilayered, multicompartment organ chip system.

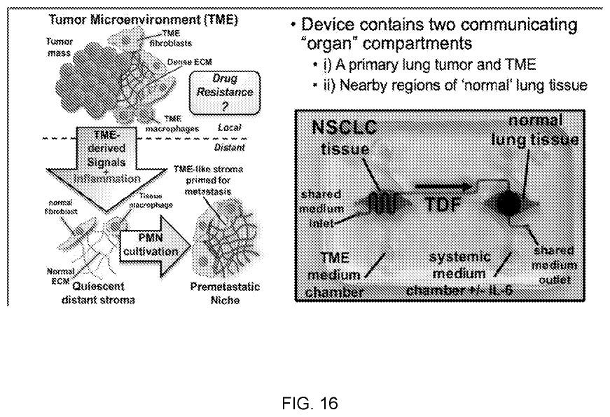

FIG. 17 shows workflow, relative scale of multilayered, multicompartment organ chip system and resulting models.

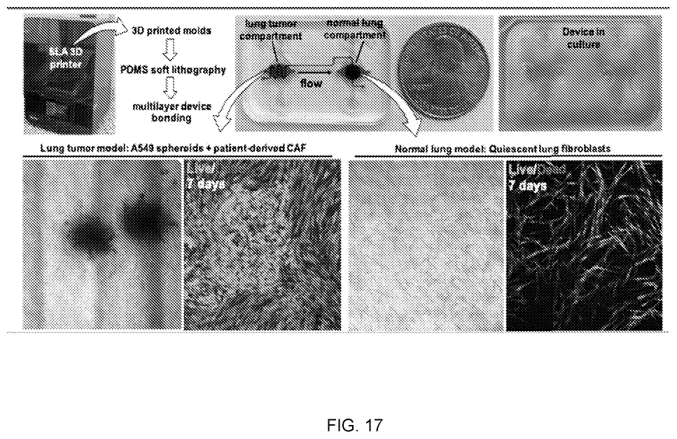

FIG. 18 shows multilayered, multicompartment organ chip system molds and device.

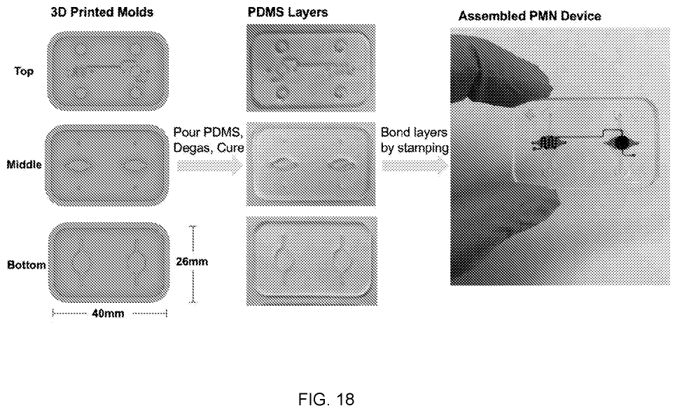

FIG. 19 shows results of aggressive cancer in tumor microenvironment in organ chip systems.

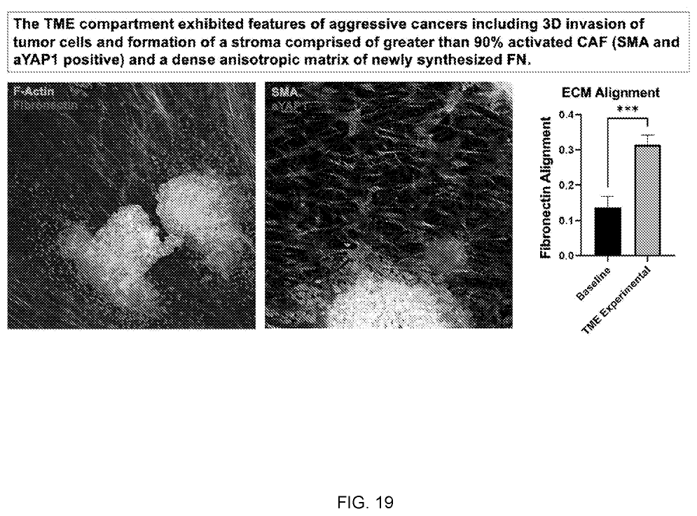

FIG. 20 shows aggressive CAF phenotypes in NSCLC tumor microenvironment.

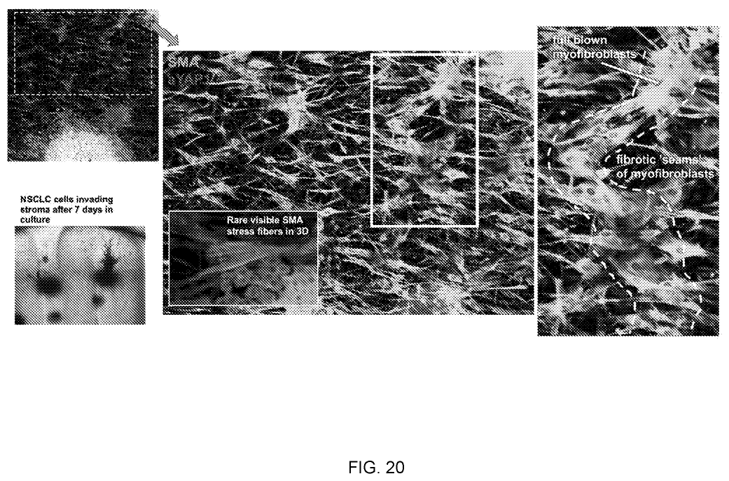

FIG. 21 shows results of stromal reaction.

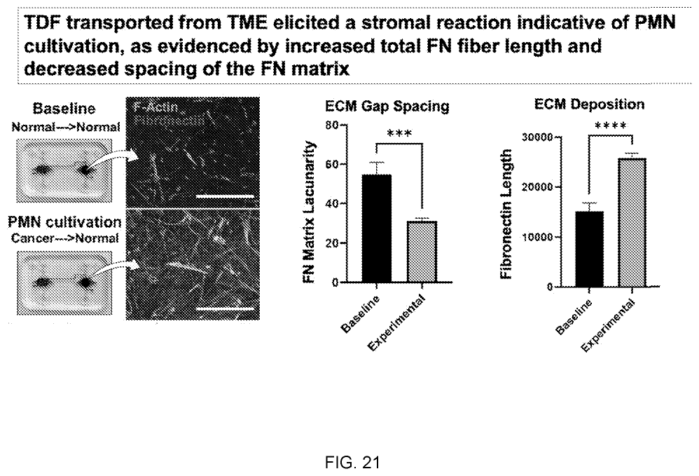

FIG. 22 shows preliminary evidence of PMN induction in model.

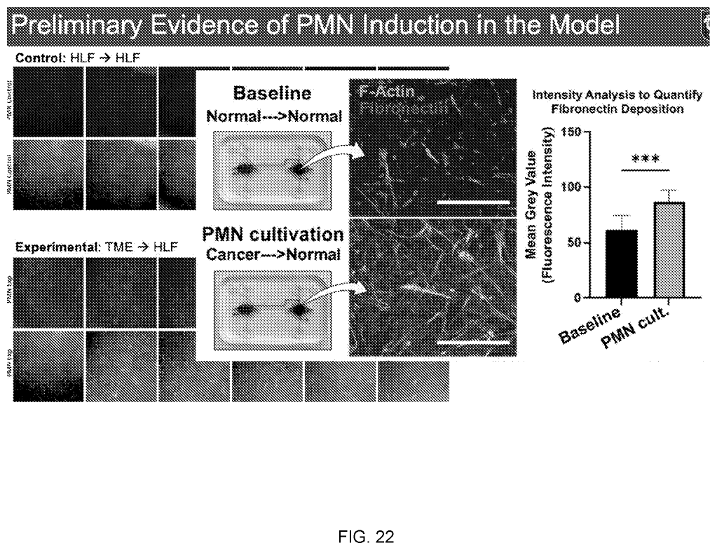

FIG. 23: Manufacturing milliscale PDMS organ chips using a low-cost SLA printer. A: Troubleshooting and post-processing workflow for organ chip manufacturing using SLA-printed molds. B: Incomplete PDMS curing in SLA-printed molds with no post-processing. C: Clamping apparatus with heated jeweler blocks for mold flattening. D: Warped 3D printed mold post curing (top) compared to the same mold after baking and flattening (bottom). E: Flattened organ chip mold for the top layer of the multilayer device in Panels G and F. F: Multilayer organ chip (3 PDMS layers, 2 membrane layers) bonded by PDMS stamping. G: Food dye loading to demonstrate device layering and patent bonding with flattened layers. H: Actin (green) staining of human lung fibroblasts growing in the 3D interstitium layer and lung adenocarcinoma cells in the top channel layer.

FIG. 24: Polyurethane clear coating of SLA-printed molds to engineer PDMS clarity. A: Schematic of the PU coating process using a handheld airbrush. B: Opaque PDMS part cast in uncoated mold (left) and optically transparent PDMS cast in clear coated mold (right). The design is a geographical homage to the Crescent City. C. Absorbance of light at varying wavelengths between clear coated and uncoated molds demonstrates the superb optical transparency of PDMS made in clear coated parts. D: Uncoated MFOC mold (left) compared to mold with polyurethane clear coat (right). E: User testing of MFOC loading success rate. Left: a failed injection defined as liquid breaching over the guides in a device manufactured using uncoated molds. Right: Example of successful injection in a device manufactured using coated molds. F: Comparison of success rate of loading between devices made using clear coated versus uncoated molds; n=6 users, 10 trials each.

FIG. 25: MFOC design iteration and reduction to practice through user testing. All testing consisted of least n=7 users performing 10 injection trials per design. A: CAD render of a prototypical MFOC device. Dimensions of internal features and volumes were varied for user testing of loading success rate. B: Drawing of cross section of single guide MFOC (top) and double guide MFOC (bottom). Key dimensions include guide height (h), tissue chamber height (H), and tissue width (W). C: Loading success rates for single guide and double guide MFOC. D: Loading success rates for various h/H values. E: Loading success rates for tissue volumes ranging from 8 to 48 mm³. F: FITC-dextran loaded collagen gel polymerized after injection loading of collagen precursor solution in an MFOC. G: Proposed free body diagram of forces acting on a liquid (collagen precursor solution for our applications) pinned between the guide structures of a double guide MFOC as shown in Panel F. F_{c} is the cohesion of the liquid and PDMS surface. F_{N} is the normal force acting at all surface contact points. Fₛₜ represents the surface tension forces stabilizing the liquid face. F_{g} is gravity and Fₘ is the inertial force tending to push the mass of liquid over the guide during injection loading.

FIG. 26: Engineering bulk tissue vasculogenesis in milliscale MFOC. A: Digital photograph of an MFOC used for bulk tissue vasculogenesis. Green lines indicate the position of guide structures that confine the bulk tissue. B: Differential interference contrast (DIC) micrograph of an MFOC loaded with HUVEC and HLF in a blend of collagen and fibrin hydrogel as described in Materials and Methods. Guide regions are marked by green coloration between the dashed lines. The cellularized bulk tissue is visible between the guide structures. C: 3D LSCM stack of a nascent vascular network in the bulk tissue after 3 days of culture. Endothelial cells are labeled with UEA-1 lectin (red). Actin in all cells is labeled with phalloidins (green). Fibroblasts are green only. Scale bar=200 μm. D: Formation of a continuous vascular network throughout the bulk tissue after 7 days of culture. Scale bar=500 μm. E: Representative stitches of UEA-1 staining in 3D LSCM stacks across the bulk tissue length used for image analyses shown in Panel G. F: Perivascular niches occupied by fibroblasts that enrobe and align along the vessel architecture after 7 days of culture. Scale bar=50 μm. G: Morphometric analysis of bulk tissue vasculogenesis. Measuring non-participating endothelial cell numbers charts the completeness of network formation. Valency charts the complexity of interconnection and branching, with lower and stable valency indicating network maturation. Increasing vessel diameter results from a combination of increased cell recruitment and lumen formation in the network. Max diffusion distance provides a measure of cell coalescence into the network and network pruning.

FIG. 27: Modeling cell migration at engineered tissue interfaces in multi-tissue MFOC. A: Digital photograph of a 2-lane MFOC designed for sequential loading. Schematic illustrating the formation of a dense interfacial layer of ECM due to sequential polymerization of the hydrogel scaffolds. B: Digital photograph of a 2-lane MFOC designed for simultaneous loading. Schematic illustrating the formation of a continuous bulk tissue with continuous ECM upon simultaneous loading and polymerization. C: Differential interference contrast (DIC) micrograph of the interfacial region on Day 0 and at Day 5 in sequentially loaded 2-lane MFOC with the presence of a dense interfacial layer (arrows). The left lane is loaded with HUVEC and HLF in a blend of collagen and fibrin hydrogel as in FIG. 26. The right lane is loaded with an acellular hydrogel of the same composition. D: DIC images of a simultaneously loaded 2-lane MFOC on Day 0 and at Day 5. E: 3D LSCM stack of the entire horizontal span of a sequentially loaded 2-lane MFOC. Endothelial cells are labeled with UEA-1 lectin (red). Actin in all cells is labeled with phalloidins (green). Fibroblasts are green only. A layer of fibroblasts is seen oriented parallel to the dense interface (arrows). Dashed white line denotes the interface. F: 3D LSCM stack of the entire horizontal span of a simultaneously loaded 2-lane MFOC. Marked invasion of fibroblasts and vascular structures not seen in sequentially loaded devices was observed. G: Counts of cell numbers per field as shown in Panels in E and F. * indicates P<0.05. N=3 devices from 2 independent experiments. All scale bars=500 microns.

FIG. 28: Modeling distant tissue interactions in 4-tissue milliscale MFOC. A: Digital photograph of the 4-tissue MFOC. See Supplemental Information for design specifications. Compartment labels indicate locations of tissues depicted in Panel B. TME=tumor microenvironment. PMN=premetastatic niche. B: Inflammatory activation visualized in 3D LSCM stacks of ICAM-1 (green), UEA-1 (endothelial cells, red), and DAPI (blue). Co-localization of UEA-1 and ICAM-1 indicates vascular inflammation (yellow). Inflammation of non-endothelial cells is localized by green only staining. Scale bars=200 μm. C: ICAM-1 fluorescence intensity in regions of colocalization with UEA-1 (Vascular ICAM-1). Data is normalized as fold increase over Control devices which replace with the TME with a fourth PMN compartment (no cancer in the system). * indicates p<0.05. D: Normalized Vascular ICAM-1 in each compartment. E: Quantifying propagation of the inflammatory signal in the bulk tissue (relative non-vascular inflammation) as a ratio of vascular to non-vascular ICAM-1 intensity discriminated based on colocalization with UEA-1. Trend lines in Panels D and E illustrate the linear gradient trends expected in a static culture system.

FIG. 29. Fabricating milliscale bulk tissues with an anastomosed internal vasculature in MFOC. A: Workflow for establishing patent and perfusable vasculature tested by FITC-dextran perfusion as described in Materials and Methods. B: Differential interference contrast (DIC) image taken at the tissue interface situated on the guide structure immediately after side channel seeding (Step 2 in Panel A, after 48 h of vasculogenesis). C: 3D LSCM stacks depicting points of vascular anastomosis at the tissue interface after 9 total days of culture. Endothelial cells are labeled with UEA-1 lectin (red). Actin in all cells is labeled with phalloidins (green). Inset: Open face of a single anastomosed vessel. D: DIC image taken at the tissue interface situated on the guide structure in a device without side channel seeding after 48 hours. E: 3D LCSM stack of the tissue interface in a device without side channel seeding after 48 hours. Inset: Dense layers of cells cover the interface and internal vascular structures remain inaccessible. 9 days of culture. F: Perfusion of 20 kDa FITC-dextran after 9 days of vascular network maturation. Green fluorescence is contained within vessels and absent in the interstitial spaces. Scale bars=100 μm.

FIG. 30—Formlabs F3 printer resolution testing. We used a Formlabs F3 SLA printer with Grey and Clear resins as described in Materials and Methods. We first determined the build resolution of the printer with these resins focusing on features commonly used in microfluidic device molds such as rectangular channel features and cylindrical features used to mold fluidic access ports. We tested the printer resolution for both positive features built above the floor of the mold and negative features fabricated as hollow impressions below the floor of the mold. Please note that these data reflect usage of the printer in our hands with the specific designs tested and do not represent the absolute limits of the printer using these materials. We measured the built feature dimensions using a stereomicroscope and calculated a percent error relative to the designed dimensions (FIG. 30A, B, E, F). We designated less than 10% error as acceptable accuracy for organ chip mold fabrication. We found that the positive feature resolution for both rectangular channels and cylindrical pillars was accurate down to sizes of 200 μm (FIG. 30C, G). These results confirmed that the printer has excellent resolution for our intended applications of manufacturing molds for millifluidic organ chip devices. Negative channel features were accurate down to 400 μm, whereas negative pillar features were inaccurate at sizes as large as 900 μm (FIG. 30D, H). These data indicate that negative feature resolution is significantly impaired relative to positive feature resolution, thereby necessitating designs that avoided negative features or implementation of a PDMS replica molding technique that enables fabrication of negative feature PDMS molds with resolution that matches the positive feature build accuracy of our SLA printer (FIG. 31).

FIG. 31—Creating PDMS replica molds to achieve negative feature molding that matches the F3 SLA printer's positive feature build accuracy. PDMS replica molding was performed as described in Methods.

FIG. 32—Surface roughness and wettability of PDMS from uncoated and PU-coated molds. We compared the surface wettability of PDMS cured on uncoated mold surfaces and PU-coated mold surfaces to better understand the result of increased loading failure rates in MFOC fabricated using uncoated molds (FIG. 24F). Differential interference contrast imaging confirmed the roughness of PDMS surfaces from uncoated molds (FIGS. 32A, B), which resulted in a visually increased hydrophobicity compared to PDMS surfaces from PU-coated molds (FIG. 32C). Increased hydrophobicity of the rough PDMS surfaces from uncoated molds was confirmed by contact angle measurements (3 replicate surfaces, 10 drops per surface) (FIGS. 32D, E). The low variance in the contact angle data confirms the consistency of surface properties of SLA-printed molds and the PU coating procedure. Statistical analysis revealed that PDMS surfaces from PU-coated molds are significantly more hydrophilic (75.1+/−1.5° vs. 102.2+/−0.6°, P<0.001).

FIG. 33 shows tumor tissues induce local and distant vascular inflammation. A. PDMS device with a tumor (TME) and three downstream organ compartments (PMN-1,2,3). Lower panels: ICAM-1 staining (green) with lectin counterstaining (red). B. Vascular ICAM-1 signal in each compartment of a single device. C. Vascular ICAM-1 for an entire series, all PMN pooled. D. Total ICAM01 for an entire series, all PMN pooled.

FIG. 34. Milliscale MFOC devices enable rapid formation of large fully vascularized TNBC explant tissues. A) MFOC device in culture and cross section rendering of the multi-guide design. B) TNBC injectable explant tissue; Ki67, 3 days in MFOC. C) 3D projection of tissue wall geometry in cross section. D) Top down view of fully anastomosed internal vasculature in MFOC at the tissue noudary region shown in Panel C, E) Fully vascularized prototype using patient-derived TNBC spheroids with perfusable vasculature spanning 1.5 mm. Refer to Panel A for reference on guide locations. 5 days. F) FITC-dextran perfusion in the device shown in Panel E.

### DETAILED DESCRIPTION

Detailed descriptions of one or more preferred embodiments are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in any appropriate manner.

### Definitions

Unless defined otherwise, technical and scientific terms used herein have the same meaning as commonly understood by one of ordinary skill in the art to which this invention belongs. For example, Singleton et al., Dictionary of Microbiology and Molecular Biology 2nd ed., J. Wiley & Sons (New York, NY 1994), provide one skilled in the art with a general guide to many of the terms used in the present application. Additionally, the practice of the present invention will employ, unless otherwise indicated, conventional techniques of molecular biology (including recombinant techniques), microbiology, cell biology, and biochemistry, which are within the skill of the art. Such techniques are explained fully in the literature, such as, “Molecular Cloning: A Laboratory Manual”, 2nd edition (Sambrook et al., 1989); “Oligonucleotide Synthesis” (M. J. Gait, ed., 1984); “Animal Cell Culture” (R. I. Freshney, ed., 1987); “Methods in Enzymology” (Academic Press, Inc.); “Handbook of Experimental Immunology”, 4th edition (D. M. Weir & C. C. Blackwell, eds., Blackwell Science Inc., 1987); “Gene Transfer Vectors for Mammalian Cells” (J. M. Miller & M. P. Calos, eds., 1987); “Current Protocols in Molecular Biology” (F. M. Ausubel et al., eds., 1987); and “PCR: The Polymerase Chain Reaction”, (Mullis et al., eds., 1994).

Wherever any of the phrases “for example,” “such as,” “including” and the like are used herein, the phrase “and without limitation” is understood to follow unless explicitly stated otherwise. Similarly “an example,” “exemplary” and the like are understood to be non-limiting.

As used in the present disclosure and claims, the singular forms “a”, “an” and “the” include plural forms unless the context clearly dictates otherwise.

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, In some embodiments, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, “either,” “one of,” “only one of,” or “exactly one of” “Consisting essentially of,” when used in the claims, shall have its ordinary meaning as used in the field of patent law.

The term “about” as used herein when referring to a measurable value such as an amount, a temporal duration, and the like, is meant to encompass variations of ±20%, ±10%, ±5%, ±1%, ±0.5%, or ±0.1% from the specified value, as such variations are appropriate to perform the disclosed methods.

The term “cancer” as used herein is meant to refer to any disease that is caused by, or results in, inappropriately high levels of cell division, inappropriately low levels of apoptosis, or both. Specific examples of cancer include, but are not limited to, Acute Lymphoblastic Leukemia, Adult; Acute Lymphoblastic Leukemia, Childhood; Acute Myeloid Leukemia, Adult; Adrenocortical Carcinoma; Adrenocortical Carcinoma, Childhood; AIDS-Related Lymphoma; AIDS-Related Malignancies; Anal Cancer; Astrocytoma, Childhood Cerebellar; Astrocytoma, Childhood Cerebral; Bile Duct Cancer, Extrahepatic; Bladder Cancer; Bladder Cancer, Childhood; Bone Cancer, Osteosarcoma/Malignant Fibrous Histiocytoma; Brain Stem Glioma, Childhood; Brain Tumor, Adult; Brain Tumor, Brain Stem Glioma, Childhood; Brain Tumor, Cerebellar Astrocytoma, Childhood; Brain Tumor, Cerebral Astrocytoma/Malignant Glioma, Childhood; Brain Tumor, Ependymoma, Childhood; Brain Tumor, Medulloblastoma, Childhood; Brain Tumor, Supratentorial Primitive Neuroectodermal Tumors, Childhood; Brain Tumor, Visual Pathway and Hypothalamic Glioma, Childhood; Brain Tumor, Childhood (Other); Breast Cancer; Breast Cancer and Pregnancy; Breast Cancer, Childhood; Breast Cancer, Male; Bronchial Adenomas/Carcinoids, Childhood: Carcinoid Tumor, Childhood; Carcinoid Tumor, Gastrointestinal; Carcinoma, Adrenocortical; Carcinoma, Islet Cell; Carcinoma of Unknown Primary; Central Nervous System Lymphoma, Primary; Cerebellar Astrocytoma, Childhood; Cerebral Astrocytoma/Malignant Glioma, Childhood; Cervical Cancer; Childhood Cancers; Chronic Lymphocytic Leukemia; Chronic Myelogenous Leukemia; Chronic Myeloproliferative Disorders; Clear Cell Sarcoma of Tendon Sheaths; Colon Cancer; Colorectal Cancer, Childhood; Cutaneous T-Cell Lymphoma; Endometrial Cancer; Ependymoma, Childhood; Epithelial Cancer, Ovarian; Esophageal Cancer; Esophageal Cancer, Childhood; Ewing's Family of Tumors; Extracranial Germ Cell Tumor, Childhood; Extragonadal Germ Cell Tumor; Extrahepatic Bile Duct Cancer; Eye Cancer, Intraocular Melanoma; Eye Cancer, Retinoblastoma; Gallbladder Cancer; Gastric (Stomach) Cancer; Gastric (Stomach) Cancer, Childhood; Gastrointestinal Carcinoid Tumor; Germ Cell Tumor, Extracranial, Childhood; Germ Cell Tumor, Extragonadal; Germ Cell Tumor, Ovarian; Gestational Trophoblastic Tumor; Glioma. Childhood Brain Stem; Glioma. Childhood Visual Pathway and Hypothalamic; Hairy Cell Leukemia; Head and Neck Cancer; Hepatocellular (Liver) Cancer, Adult (Primary); Hepatocellular (Liver) Cancer, Childhood (Primary); Hodgkin's Lymphoma, Adult; Hodgkin's Lymphoma, Childhood; Hodgkin's Lymphoma During Pregnancy; Hypopharyngeal Cancer; Hypothalamic and Visual Pathway Glioma, Childhood; Intraocular Melanoma; Islet Cell Carcinoma (Endocrine Pancreas); Kaposi's Sarcoma; Kidney Cancer; Laryngeal Cancer; Laryngeal Cancer, Childhood; Leukemia, Acute Lymphoblastic, Adult; Leukemia, Acute Lymphoblastic, Childhood; Leukemia, Acute Myeloid, Adult; Leukemia, Acute Myeloid, Childhood; Leukemia, Chronic Lymphocytic; Leukemia, Chronic Myelogenous; Leukemia, Hairy Cell; Lip and Oral Cavity Cancer; Liver Cancer, Adult (Primary); Liver Cancer, Childhood (Primary); Lung Cancer, Non-Small Cell; Lung Cancer, Small Cell; Lymphoblastic Leukemia, Adult Acute; Lymphoblastic Leukemia, Childhood Acute; Lymphocytic Leukemia, Chronic; Lymphoma, AIDS-Related; Lymphoma, Central Nervous System (Primary); Lymphoma, Cutaneous T-Cell; Lymphoma, Hodgkin's, Adult; Lymphoma, Hodgkin's; Childhood; Lymphoma, Hodgkin's During Pregnancy; Lymphoma, Non-Hodgkin's, Adult; Lymphoma, Non-Hodgkin's, Childhood; Lymphoma, Non-Hodgkin's During Pregnancy; Lymphoma, Primary Central Nervous System; Macroglobulinemia, Waldenstrom's; Male Breast Cancer; Malignant Mesothelioma, Adult; Malignant Mesothelioma, Childhood; Malignant Thymoma; Medulloblastoma, Childhood; Melanoma; Melanoma, Intraocular; Merkel Cell Carcinoma; Mesothelioma, Malignant; Metastatic Squamous Neck Cancer with Occult Primary; Multiple Endocrine Neoplasia Syndrome, Childhood; Multiple Myeloma/Plasma Cell Neoplasm; Mycosis Fungoides; Myelodysplasia Syndromes; Myelogenous Leukemia, Chronic; Myeloid Leukemia, Childhood Acute; Myeloma, Multiple; Myeloproliferative Disorders, Chronic; Nasal Cavity and Paranasal Sinus Cancer; Nasopharyngeal Cancer; Nasopharyngeal Cancer, Childhood; Neuroblastoma; Neurofibroma; Non-Hodgkin's Lymphoma, Adult; Non-Hodgkin's Lymphoma, Childhood; Non-Hodgkin's Lymphoma During Pregnancy; Non-Small Cell Lung Cancer; Oral Cancer, Childhood; Oral Cavity and Lip Cancer; Oropharyngeal Cancer; Osteosarcoma/Malignant Fibrous Histiocytoma of Bone; Ovarian Cancer, Childhood; Ovarian Epithelial Cancer; Ovarian Germ Cell Tumor; Ovarian Low Malignant Potential Tumor; Pancreatic Cancer; Pancreatic Cancer, Childhood', Pancreatic Cancer, Islet Cell; Paranasal Sinus and Nasal Cavity Cancer; Parathyroid Cancer; Penile Cancer; Pheochromocytoma; Pineal and Supratentorial Primitive Neuroectodermal Tumors, Childhood; Pituitary Tumor; Plasma Cell Neoplasm/Multiple Myeloma; Pleuropulmonary Blastoma; Pregnancy and Breast Cancer; Pregnancy and Hodgkin's Lymphoma; Pregnancy and Non-Hodgkin's Lymphoma; Primary Central Nervous System Lymphoma; Primary Liver Cancer, Adult; Primary Liver Cancer, Childhood; Prostate Cancer; Rectal Cancer; Renal Cell (Kidney) Cancer; Renal Cell Cancer, Childhood; Renal Pelvis and Ureter, Transitional Cell Cancer; Retinoblastoma; Rhabdomyosarcoma, Childhood; Salivary Gland Cancer; Salivary Gland Cancer, Childhood; Sarcoma, Ewing's Family of Tumors; Sarcoma, Kaposi's; Sarcoma (Osteosarcoma)/Malignant Fibrous Histiocytoma of Bone; Sarcoma, Rhabdomyosarcoma, Childhood; Sarcoma, Soft Tissue, Adult; Sarcoma, Soft Tissue, Childhood; Sezary Syndrome; Skin Cancer; Skin Cancer, Childhood; Skin Cancer (Melanoma); Skin Carcinoma, Merkel Cell; Small Cell Lung Cancer; Small Intestine Cancer; Soft Tissue Sarcoma, Adult; Soft Tissue Sarcoma, Childhood; Squamous Neck Cancer with Occult Primary, Metastatic; Stomach (Gastric) Cancer; Stomach (Gastric) Cancer, Childhood; Supratentorial Primitive Neuroectodermal Tumors, Childhood; T-Cell Lymphoma, Cutaneous; Testicular Cancer; Thymoma, Childhood; Thymoma, Malignant; Thyroid Cancer; Thyroid Cancer, Childhood; Transitional Cell Cancer of the Renal Pelvis and Ureter; Trophoblastic Tumor, Gestational; Unknown Primary Site, Cancer of, Childhood; Unusual Cancers of Childhood; Ureter and Renal Pelvis, Transitional Cell Cancer; Urethral Cancer; Uterine Sarcoma; Vaginal Cancer; Visual Pathway and Hypothalamic Glioma, Childhood; Vulvar Cancer; Waldenstrom's Macro globulinemia; and Wilms' Tumor. In some embodiments, the cancer cells comprise or consist of one of the above-identified cancer cell types. In some embodiments, the cells are transformed cells that are from one of the above-identified cancer lineages.

By “fragment” is meant a portion of a polypeptide or nucleic acid molecule. This portion contains, preferably, at least 5%, 10%, 20%, 30%, 40%, 50%, 60%, 70%, 80%, or 90% of the entire length of the reference nucleic acid molecule or polypeptide. A fragment may contain 5, 10, 20, 30, 40, 50, 60, 70, 80, 90, or 100, 200, 300, 400, 500, 600, 700, 800, 900, 1000 or more nucleotides or amino acids.

As used herein, the terms “prevent,” “preventing,” “prevention,” “prophylactic treatment,” and the like, are meant to refer to reducing the probability of developing a disease or condition in a subject, who does not have, but is at risk of or susceptible to developing a disease or condition.

As used herein, the term “environmental agent” refers to a class of small molecules present in environment that is tested for its capacity to accelerate or inhibit the growth, differentiation or vascularization of cancer cells.

As used herein, the term “small molecule” refers to a low molecular weight (<900 daltons) organic compound that may help regulate a biological process, with a size on the order of 10 9 m. Most drugs are small molecules.

As used herein, the term “biologic immunotherapy” refers to biologic materials such as antibodies, antibody-based fragments, and antibody-drug conjugates.

As used herein, the term “modified T cell” refers to genetically engineered lymphocytes comprising a T-cell receptor on their cell surface.

As used herein, the term chimeric antigen receptor (CAR) T-cells refers to a modified T-cells engineered to target antigens expressed on cancer cells.

The term “substantially” allows for deviations from the descriptor that do not negatively impact the intended purpose. Descriptive terms are understood to be modified by the term “substantially” even if the word “substantially” is not explicitly recited. Therefore, for example, the phrase “wherein the lever extends vertically” means “wherein the lever extends substantially vertically” so long as a precise vertical arrangement is not necessary for the lever to perform its function.

The term “culture vessel” as used herein is defined as any vessel suitable for growing, culturing, cultivating, proliferating, propagating, or otherwise similarly manipulating cells. A culture vessel may also be referred to herein as a “culture insert”. In some embodiments, the culture vessel is made out of biocompatible plastic and/or glass. In some embodiments, the plastic is a thin layer of plastic comprising one or a plurality of pores that allow diffusion of protein, nucleic acid, nutrients (such as heavy metals and hormones) antibiotics, and other cell culture medium components through the pores. In some embodiments, the pores are not more than about 0.1, 0.5 1.0, 2, 3, 4, 5, 6, 7, 8, 9, 10, 15, 20, 25, 30, 40, 50 microns wide. In some embodiments, the culture vessel in a hydrogel matrix and free of a base or any other structure. In some embodiments, the culture vessel is designed to contain a hydrogel or hydrogel matrix and various culture mediums. In some embodiments, the culture vessel consists of or consists essentially of a hydrogel or hydrogel matrix. In some embodiments, the only plastic component of the culture vessel is the components of the culture vessel that make up the side walls and/or bottom of the culture vessel that separate the volume of a well or zone of cellular growth from a point exterior to the culture vessel. In some embodiments, the culture vessel comprises a hydrogel and one or a plurality of isolated stromal cells. In some embodiments, the culture vessel comprises a hydrogel and one or a plurality of isolated stromal cells, to which one or a plurality of cancer cells are seeded.

The term “exposing” as used herein refers to bringing a disclosed compound and a cell, target receptor, or other biological entity together in direct or indirect contact, in such a manner that the compound can affect the activity of the cell (e.g., receptor, cell, etc.). Directly this can occur by physical contact between the disclosed compound and the cell, receptor o other entity; i.e., by interacting with the target or cell itself, or indirectly this can occur by interacting with another molecule, co-factor, factor, or protein on which the activity of the cell is dependent. In some embodiments, the activity of the cell in response to the compound or molecule is differentiation. In some embodiments, the compound is one or more differentiation factors, therapeutic agents or therapeutic agent candidate.

“Analogues” of the compounds disclosed herein are pharmaceutically acceptable salts, prodrugs, deuterated forms, radio-actively labeled forms, isomers, solvates and combinations thereof. The “combinations” mentioned in this context are refer to derivatives falling within at least two of the groups: pharmaceutically acceptable salts, prodrugs, deuterated forms, radio-actively labeled forms, isomers, and solvates. Examples of radio-actively labeled forms include compounds labeled with tritium, phosphorous-32, iodine-129, carbon-11, fluorine-18, and the like. The compounds described herein may be present in the form of pharmaceutically acceptable salts. For use in medicines, the salts of the compounds described herein refer to non-toxic “pharmaceutically acceptable salts.” Pharmaceutically acceptable salt forms include pharmaceutically acceptable acidic/anionic or basic/cationic salts. Suitable pharmaceutically acceptable acid addition salts of the compounds described herein include e.g., salts of inorganic acids (such as hydrochloric acid, hydrobromic, phosphoric, nitric, and sulfuric acids) and of organic acids (such as, acetic acid, benzenesulfonic, benzoic, methanesulfonic, and p-toluenesulfonic acids). Examples of pharmaceutically acceptable base addition salts include e.g., sodium, potassium, calcium, ammonium, organic amino, or magnesium salt. As used herein, the term “salt” refers to acid or base salts of the compounds used in the methods of the present disclosure. Illustrative examples of acceptable salts are mineral acid (hydrochloric acid, hydrobromic acid, phosphoric acid, and the like) salts, organic acid (acetic acid, propionic acid, glutamic acid, citric acid and the like) salts, quaternary ammonium (methyl iodide, ethyl iodide, and the like) salts.

As defined herein, the term “inhibition,” “inhibit,” “inhibiting,” and the like in reference to a protein-inhibitor (e.g., antagonist) interaction means negatively affecting (e.g., decreasing) the activity or function of the protein relative to the activity or function of the protein in the absence of the inhibitor. In embodiments inhibition refers to reduction of a disease or symptoms of disease. In embodiments, inhibition refers to a reduction in the activity of a signal transduction pathway or signaling pathway. Thus, inhibition includes, at least in part, partially or totally blocking stimulation, decreasing, preventing, or delaying activation, or inactivating, desensitizing, or down-regulating signal transduction or enzymatic activity or the amount of a protein.

The term “hydrogel” as used herein is defined as any water-insoluble, crosslinked, three-dimensional network of polymer chains with the voids between polymer chains filled with or capable of being filled with water. The term “hydrogel matrix” as used herein is defined as any three-dimensional hydrogel construct, system, device, or similar structure. In some embodiments, the hydrogel or hydrogel matrix comprises one or more proteins and/or glycoproteins. In some embodiments, the hydrogel or hydrogel matrix comprises one or more of the following proteins: collagen, gelatin, elastin, titin, laminin, fibronectin, fibrin, keratin, silk fibroin, and any derivatives or combinations thereof. In some embodiments, the hydrogel or hydrogel matrix comprises Matrigel® or vitronectin. In some embodiments, the hydrogel or hydrogel matrix can be solidified into various shapes. In some embodiments, the hydrogel or hydrogel matrix comprises poly (ethylene glycol) dimethacrylate (PEG). In some embodiments, the hydrogel or hydrogel matrix comprises Puramatrix. In some embodiments, the hydrogel or hydrogel matrix comprises glycidyl methacrylate-dextran (MeDex). In some embodiments, two or more hydrogels or hydrogel matrixes are used simultaneously cell culture vessel. In some embodiments, two or more hydrogels or hydrogel matrixes are used simultaneously in the same cell culture vessel but the hydrogels are separated by a wall that create independently addressable microenvironments in the tissue culture vessel such as wells. In a multiplexed tissue culture vessel it is possible for some embodiments to include any number of aforementioned wells or independently addressable location within the cell culture vessel such that a hydrogel matrix in one well or location is different or the same as the hydrogel matrix in another well or location of the cell culture vessel.

The term “Matrigel®” means a solubilized basement membrane preparation extracted from the Engelbreth-Holm-Swarm (EHS) mouse sarcoma comprising ECM proteins comprising laminin, collagen IV, heparin sulfate proteoglycans, and entactin/nidogen. In some embodiments, Cultrex® BME (Trevigen, Inc.) or Geltrex® (Thermo-Fisher Inc.) may be substituted for Matrigel®.

In some embodiments, the hydrogel or hydrogel matrices can have various thicknesses. In some embodiments, the thickness of the hydrogel or hydrogel matrix is from about 10 μm to about 3000 μm. In some embodiments, the thickness of the hydrogel or hydrogel matrix is from about 150 μm to about 3000 μm. In some embodiments, the thickness of the hydrogel or hydrogel matrix is from about 200 μm to about 3000 μm. In some embodiments, the thickness of the hydrogel or hydrogel matrix is from about 250 μm to about 3000 μm. In some embodiments, the thickness of the hydrogel or hydrogel matrix is from about 300 μm to about 3000 μm. In some embodiments, the thickness of the hydrogel or hydrogel matrix is from about 350 μm to about 3000 μm. In some embodiments, the thickness of the hydrogel or hydrogel matrix is from about 400 μm to about 3000 μm. In some embodiments, the thickness of the hydrogel or hydrogel matrix is from about 450 μm to about 3000 μm. In some embodiments, the thickness of the hydrogel or hydrogel matrix is from about 500 μm to about 3000 μm. In some embodiments, the thickness of the hydrogel or hydrogel matrix is from about 550 μm to about 3000 μm. In some embodiments, the thickness of the hydrogel or hydrogel matrix is from about 600 μm to about 3000 μm. In some embodiments, the thickness of the hydrogel or hydrogel matrix is from about 650 μm to about 3000 μm. In some embodiments, the thickness of the hydrogel or hydrogel matrix is from about 700 μm to about 3000 μm. In some embodiments, the thickness of the hydrogel or hydrogel matrix is from about 750 μm to about 3000 μm. In some embodiments, the thickness of the hydrogel or hydrogel matrix is from about 800 μm to about 3000 μm. In some embodiments, the thickness of the hydrogel or hydrogel matrix is from about 850 μm to about 3000 μm. In some embodiments, the thickness of the hydrogel or hydrogel matrix is from about 900 μm to about 3000 μm. In some embodiments, the thickness of the hydrogel or hydrogel matrix is from about 950 μm to about 3000 μm. In some embodiments, the thickness of the hydrogel or hydrogel matrix is from about 1000 μm to about 3000 μm. In some embodiments, the thickness of the hydrogel or hydrogel matrix is from about 1500 μm to about 3000 μm. In some embodiments, the thickness of the hydrogel or hydrogel matrix is from about 2000 μm to about 3000 μm. In some embodiments, the thickness of the hydrogel or hydrogel matrix is from about 2500 μm to about 3000 μm. In some embodiments, the thickness of the hydrogel or hydrogel matrix is from about 100 μm to about 2500 μm. In some embodiments, the thickness of the hydrogel or hydrogel matrix is from about 100 μm to about 2000 μm. In some embodiments, the thickness of the hydrogel or hydrogel matrix is from about 100 μm to about 1500 μm. In some embodiments, the thickness of the hydrogel or hydrogel matrix is from about 100 μm to about 1000 μm. In some embodiments, the thickness of the hydrogel or hydrogel matrix is from about 100 μm to about 950 μm. In some embodiments, the thickness of the hydrogel or hydrogel matrix is from about 100 μm to about 900 μm. In some embodiments, the thickness of the hydrogel or hydrogel matrix is from about 100 μm to about 850 μm. In some embodiments, the thickness of the hydrogel or hydrogel matrix is from about 100 μm to about 800 μm. In some embodiments, the thickness of the hydrogel or hydrogel matrix is from about 100 μm to about 750 μm. In some embodiments, the thickness of the hydrogel or hydrogel matrix is from about 100 μm to about 700 μm. In some embodiments, the thickness of the hydrogel or hydrogel matrix is from about 100 μm to about 650 μm. In some embodiments, the thickness of the hydrogel or hydrogel matrix is from about 100 μm to about 600 μm. In some embodiments, the thickness of the hydrogel or hydrogel matrix is from about 100 μm to about 550 μm. In some embodiments, the thickness of the hydrogel or hydrogel matrix is from about 100 μm to about 500 μm. In some embodiments, the thickness of the hydrogel or hydrogel matrix is from about 100 μm to about 450 μm. In some embodiments, the thickness of the hydrogel or hydrogel matrix is from about 100 μm to about 400 μm. In some embodiments, the thickness of the hydrogel or hydrogel matrix is from about 100 μm to about 350 μm. In some embodiments, the thickness of the hydrogel or hydrogel matrix is from about 100 μm to about 300 μm. In some embodiments, the thickness of the hydrogel or hydrogel matrix is from about 100 μm to about 250 μm. In some embodiments, the thickness of the hydrogel or hydrogel matrix is from about 100 μm to about 200 μm. In some embodiments, the thickness of the hydrogel or hydrogel matrix is from about 100 μm to about 150 μm. In some embodiments, the thickness of the hydrogel or hydrogel matrix is from about 300 μm to about 600 μm. In some embodiments, the thickness of the hydrogel or hydrogel matrix is from about 400 μm to about 500 μm.

In some embodiments, the hydrogel or hydrogel matrix comprises one or more synthetic polymers. In some embodiments, the hydrogel or hydrogel matrix comprises one or more of the following synthetic polymers: polyethylene glycol (polyethylene oxide), polyvinyl alcohol, poly-2-hydroxyethyl methacrylate, polyacrylamide, silicones, and any derivatives or combinations thereof.

In some embodiments, the hydrogel or hydrogel matrix comprises one or more synthetic and/or natural polysaccharides. In some embodiments, the hydrogel or hydrogel matrix comprises one or more of the following polysaccharides: hyaluronic acid, heparin sulfate, heparin, dextran, agarose, chitosan, alginate, and any derivatives or combinations thereof.

In some embodiments, the hydrogel or hydrogel matrix comprises one or more proteins and/or glycoproteins. In some embodiments, the hydrogel or hydrogel matrix comprises one or more of the following proteins: collagen, gelatin, elastin, titin, laminin, fibronectin, fibrin, keratin, silk fibroin, and any derivatives or combinations thereof.

The term “biomarker” as used herein refers to a biological molecule present in an individual or on the surface of a call at varying concentrations useful for determining a phenotype of the cell. A biomarker may include but is not limited to, nucleic acids, proteins and variants and fragments thereof. A biomarker may be DNA comprising the entire or partial nucleic acid sequence encoding the biomarker, or the complement of such a sequence. In some embodiments, the biomarker is a mRNA expression pattern Biomarker nucleic acids useful in the invention are considered to include both DNA and RNA comprising the entire or partial sequence of any of the nucleic acid sequences of interest.

The term “two-dimensional culture” as used herein is defined as cultures of cells that lie flat on hydrogels, including Matrigel® and vitronectin, disposed in culture vessels with only a one to four cell height. In some embodiments, two-dimensional culture is not more than 3 cells high. In some embodiments, two-dimensional culture is not more than 2 cells high. In some embodiments, two-dimensional culture is not more than 1 cell high.

As used herein, a “spheroid” or “cell spheroid” means any grouping of cells in a three-dimensional shape that generally corresponds to an oval or circle rotated about one of its principal axes, major or minor, and includes three-dimensional egg shapes, oblate and prolate spheroids, spheres, and substantially equivalent shapes.

A spheroid of the present disclosure can have any suitable width, length, thickness, and/or diameter. In some embodiments, a spheroid may have a width, length, thickness, and/or diameter in a range from about 10 μm to about 50,000 μm, or any range therein, such as, but not limited to, from about 10 μm to about 900 μm, about 100 μm to about 700 μm, about 300 μm to about 600 μm, about 400 μm to about 500 μm, about 500 m to about 1,000 μm, about 600 μm to about 1,000 μm, about 700 μm to about 1,000 μm, about 800 μm to about 1,000 μm, about 900 μm to about 1,000 μm, about 750 μm to about 1,500 μm, about 1,000 μm to about 5,000 μm, about 1,000 μm to about 10,000 μm, about 2,000 to about 50,000 μm, about 25,000 μm to about 40,000 μm, or about 3,000 μm to about 15,000 μm. In some embodiments, a spheroid may have a width, length, thickness, and/or diameter of about 50 μm, 100 μm, 200 μm, 300 μm, 400 μm, 500 μm, 600 μm, 700 μm, 800 μm, 900 μm, 1,000 μm, 5,000 μm, 10,000 μm, 20,000 μm, 30,000 pam, 40,000 μm, or 50,000 μm. In some embodiments, a plurality of spheroids are generated, and each of the spheroids of the plurality may have a width, length, thickness, and/or diameter that varies by less than about 20%, such as, for example, less than about 15%, 10%, or 5%. In some embodiments, each of the spheroids of the plurality may have a different width, length, thickness, and/or diameter within any of the ranges set forth above.

The cells in a spheroid may have a particular orientation. In some embodiments, the spheroid may comprise an interior core and an exterior surface. In some embodiments, the spheroid may be hollow (i.e., may not comprise cells in the interior). In some embodiments, the interior core cells and the exterior surface cells are different types of cell.

In some embodiments, spheroids may be made up of one, two, three or more different cell types, including one or a plurality of cancer cell types and/or one or a plurality of stem cell types. In some embodiments, the interior core cells comprise one, two, three, or more different cell types. In some embodiments, the interior core cells comprise cancer cells. In some embodiments, the interior core cells comprise carcinoma cells. In some embodiments, the exterior surface cells comprise one, two, three, or more different cell types. In some embodiments, the exterior surface cells comprise stromal cells. In some embodiments, the exterior surface cells comprise fibroblasts.

In some embodiments, the spheroids comprise at least two types of cells. In some embodiments the spheroids comprise stromal cells and cancer cells. In some embodiments, the spheroids comprise stromal cells and cancer cells at a ratio of about 5:1, 4:1, 3:1, 2:1 or 1:1 of stromal cells to cancer cells. In some embodiments, the spheroids comprise stromal cells and cancer cells at a ratio of about 5:1, 4:1, 3:1, 2:1 or 1:1. In some embodiments, the spheroids comprise stromal cells and cancer cells at a ratio of about 1:5: 1:4, 1:3, or 1:2. Any combination of cell types disclosed herein may be used in the above-identified ratios within the spheroids of the disclosure.

Depending on the particular embodiment, groups of cells may be placed according to any suitable shape, geometry, and/or pattern. For example, independent groups of cells may be deposited as spheroids, and the spheroids may be arranged within a three dimensional grid, or any other suitable three dimensional pattern. The independent spheroids may all comprise approximately the same number of cells and be approximately the same size, or alternatively, different spheroids may have different numbers of cells and different sizes. In some embodiments, multiple spheroids may be arranged in shapes such as an L or T shape, radially from a single point or multiple points, sequential spheroids in a single line or parallel lines, tubes, cylinders, toroids, hierarchically branched vessel networks, high aspect ratio objects, thin closed shells, organoids, or other complex shapes which may correspond to geometries of tissues, vessels or other biological structures. In some embodiments, the compositions comprise spheroids comprising cancer cells wrapped with or covered by a plurality of compressed and highly dense stromal cells. In some embodiments, the stromal cells are constrained in growth only by the surface tension of the cell growth and are free of tension caused by an external point of tension or point of tension other than natural tension exerted by the three-dimensional culture of cells.

The term “subject” as used herein refers to any animal (e.g., a mammal), including, but not limited to, humans, non-human primates, canines, felines, rodents, and the like. Preferably, the subject is a human subject. The terms “subject,” “individual,” and “patient” are used interchangeably herein. The terms “subject,” “individual,” and “patient” thus encompass individuals having disorders such as cancer, for example, carcinoma.

As used herein, the term “therapeutic” means an agent utilized to treat, combat, ameliorate, prevent or improve an unwanted condition or disease of a patient.

A “therapeutically effective amount” or “effective amount” of a composition is a predetermined amount calculated to achieve the desired effect, i.e., to treat, combat, ameliorate, prevent or improve one or more symptoms of a viral infection. The activity contemplated by the present methods includes both medical therapeutic and/or prophylactic treatment, as appropriate. The specific dose of a compound administered according to the present disclosure to obtain therapeutic and/or prophylactic effects will, of course, be determined by the particular circumstances surrounding the case, including, for example, the compound administered, the route of administration, and the condition being treated. It will be understood that the effective amount administered will be determined by the physician in the light of the relevant circumstances including the condition to be treated, the choice of compound to be administered, and the chosen route of administration, and therefore the above dosage ranges are not intended to limit the scope of the present disclosure in any way. A therapeutically effective amount of compounds of embodiments of the present disclosure is typically an amount such that when it is administered in a physiologically tolerable excipient composition, it is sufficient to achieve an effective systemic concentration or local concentration in the tissue.

For any therapeutic agent described herein the therapeutically effective amount may be initially determined from preliminary in vitro studies and/or animal models. A therapeutically effective dose may also be determined from human data. The applied dose may be adjusted based on the relative bioavailability and potency of the administered agent. Adjusting the dose to achieve maximal efficacy based on the methods described above and other well-known methods is within the capabilities of the ordinarily skilled artisan. General principles for determining therapeutic effectiveness, which may be found in Chapter 1 of Goodman and Gilman's The Pharmacological Basis of Therapeutics, 10th Edition, McGraw-Hill (New York) (2001), incorporated herein by reference.

As used herein, the terms “treat,” “treated,” or “treating” can refer to therapeutic treatment and/or prophylactic or preventative measures wherein the object is to prevent or slow down (lessen) an undesired physiological condition, disorder or disease, or obtain beneficial or desired clinical results. For purposes of the embodiments described herein, beneficial or desired clinical results include, but are not limited to, alleviation of symptoms; diminishment of extent of condition, disorder or disease; stabilized (i.e., not worsening) state of condition, disorder or disease; delay in onset or slowing of condition, disorder or disease progression; amelioration of the condition, disorder or disease state or remission (whether partial or total), whether detectable or undetectable; an amelioration of at least one measurable physical parameter, not necessarily discernible by the patient; or enhancement or improvement of condition, disorder or disease. Treatment can also include eliciting a clinically significant response without excessive levels of side effects. Treatment also includes prolonging survival as compared to expected survival if not receiving treatment.

“Treating” includes the concepts of “alleviating”, which refers to lessening the frequency of occurrence or recurrence, or the severity, of any symptoms or other ill effects related to a cancer and/or the side effects associated with cancer therapy. The term “treating” also encompasses the concept of “managing” which refers to reducing the severity of a particular disease or disorder in a patient or delaying its recurrence, e.g., lengthening the period of remission in a patient who had suffered from the disease. It is appreciated that, although not precluded, treating a disorder or condition does not require that the disorder, condition, or symptoms associated therewith be completely eliminated.

As used herein, the term “treating cancer” is not intended to be an absolute term. In some aspects, the compositions and methods of the invention seek to reduce the size of a tumor or number of cancer cells, cause a cancer to go into remission, or prevent growth in size or cell number of cancer cells. In some circumstances, treatment with the leads to an improved prognosis.

The term “preventing” or “prevention” or “prevent” as used herein refers to prophylactic or preventative measures that prevent or slow the development of a targeted pathologic condition or disorder. Those in need of treatment include those already diagnosed with the disorder; those prone to have the disorder; and those in whom the disorder is to be prevented.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. The term “about” as used herein when referring to a measurable value such as an amount, a temporal duration, and the like, is meant to encompass variations of ±20%, ±10%, ±5%, ±1%, ±0.5%, or ±0.1% from the specified value, as such variations are appropriate to perform the disclosed methods.

The “percent identity” or “percent homology” of two polynucleotide or two polypeptide sequences is determined by comparing the sequences using the GAP computer program (a part of the GCG Wisconsin Package, version 10.3 (Accelrys, San Diego, Calif)) using its default parameters. “Identical” or “identity” as used herein in the context of two or more nucleic acids or amino acid sequences, may mean that the sequences have a specified percentage of residues that are the same over a specified region. The percentage may be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. When comparing DNA and RNA, thymine (T) and uracil (U) may be considered equivalent. Identity may he performed manually or by using a computer sequence algorithm such as BLAST or BLAST 2.0. Briefly, the BLAST algorithm, which stands for Basic Local Alignment Search Tool is suitable for determining sequence similarity. Software for performing BLAST analyses is publicly available through the National Center for Biotechnology Information (http://www.ncbi.nlm.nih.gov). This algorithm involves first identifying high scoring sequence pair (HSPs) by identifying short words of length Win the query sequence that either match or satisfy some positive-valued threshold score T when aligned with a word of the same length in a database sequence. T is referred to as the neighborhood word score threshold (Altschul et al., supra). These initial neighborhood word hits act as seeds for initiating searches to find HSPs containing them. The word hits are extended in both directions along each sequence for as far as the cumulative alignment score can be increased. Extension for the word hits in each direction are halted when: 1) the cumulative alignment score falls off by the quantity X from its maximum achieved value; 2) the cumulative score goes to zero or below, due to the accumulation of one or more negative-scoring residue alignments; or 3) the end of either sequence is reached. The Blast algorithm parameters W, T and X determine the sensitivity and speed of the alignment. The Blast program uses as defaults a word length (W) of 11, the BLOSUM62 scoring matrix (see Henikoff et al., Proc. Natl. Acad. Sci. USA, 1992, 89, 10915-10919, which is incorporated herein by reference in its entirety) alignments (B) of 50, expectation (E) of 10, M=5, N=4, and a comparison of both strands. The BLAST algorithm (Karlin et al., Proc. Natl. Acad. Sci. USA, 1993, 90, 5873-5787, which is incorporated herein by reference in its entirety) and Gapped BLAST perform a statistical analysis of the similarity between two sequences. One measure of similarity provided by the BLAST algorithm is the smallest sum probability (P(N)), which provides an indication of the probability by which a match between two nucleotide sequences would occur by chance. For example, a nucleic acid is considered similar to another if the smallest sum probability in comparison of the test nucleic acid to the other nucleic acid is less than about 1, less than about 0.1, less than about 0.01, and less than about 0.001. Two single-stranded polynucleotides are “the complement” of each other if their sequences can be aligned in an anti-parallel orientation such that every nucleotide in one the introduction of gaps, and without unpaired nucleotides at the 5′ or the 3′ end of either sequence. A polynucleotide is “complementary” to another polynucleotide if the two polynucleotides can hybridize to one another under moderately stringent conditions. Thus, a polynucleotide can be complementary to another polynucleotide without being its complement.

The terms “functional fragment” means any portion of a polypeptide or nucleic acid sequence from which the respective full-length polypeptide or nucleic acid relates that is of a sufficient length and has a sufficient structure to confer a biological affect that is at least similar or substantially similar to the full-length polypeptide or nucleic acid upon which the fragment is based. In some embodiments, a functional fragment is a portion of a full-length or wild-type nucleic acid sequence that encodes any one of the nucleic acid sequences disclosed herein, and said portion encodes a polypeptide of a certain length and/or structure that is less than full-length but encodes a domain that still biologically functional as compared to the full-length or wild-type protein. In some embodiments, the functional fragment may have a reduced biological activity, about equivalent biological activity, or an enhanced biological activity as compared to the wild-type or full-length polypeptide sequence upon which the fragment is based. In some embodiments, the functional fragment is derived from the sequence of an organism, such as a human. In such embodiments, the functional fragment may retain 99%, 98%, 97%, 96%, 95%, 94%, 93%, 92%, 91%, or 90% sequence identity to the wild-type human sequence upon which fragment is derived. In some embodiments, the functional fragment may retain 85%, 80%, 75%, 70%, 65%, or 60% sequence identity to the wild-type sequence upon which the sequence is derived.

By “fragment” is meant a portion of a polypeptide or nucleic acid molecule. This portion contains, preferably, at least about 5%, 10%, 20%, 30%, 40%, 50%, 60%, 70%, 80%, or about 90% of the entire length of the reference nucleic acid molecule or polypeptide. A fragment may contain about 5, 10, 20, 30, 40, 50, 60, 70, 80, 90, or 100, 200, 300, 400, 500, 600, 700, 800, 900, 1000 or more nucleotides or amino acids.

The terms “polypeptide”, “peptide” and “protein” are used interchangeably herein to refer to polymers of amino acids of any length. The polymer may be linear or branched, it may comprise modified amino acids, and it may be interrupted by non-natural amino acids or chemical groups that are not amino acids. The terms also encompass an amino acid polymer that has been modified; for example, disulfide bond formation, glycosylation, lipidation, acetylation, phosphorylation, or any other manipulation, such as conjugation with a labeling component. As used herein the term “amino acid” includes natural and/or unnatural or synthetic amino acids, including glycine and both the D or L optical isomers, and amino acid analogs and peptidomimetics.

“Variants” is intended to mean substantially similar sequences. For nucleic acid molecules, a variant comprises a nucleic acid molecule having deletions (i.e., truncations) at the 5′ and/or 3′ end; deletion and/or addition of one or more nucleotides at one or more internal sites in the native polynucleotide; and/or substitution of one or more nucleotides at one or more sites in the native polynucleotide. As used herein, a “native” nucleic acid molecule or polypeptide comprises a naturally occurring nucleotide sequence or amino acid sequence, respectively. For nucleic acid molecules, conservative variants include those sequences that, because of the degeneracy of the genetic code, encode the amino acid sequence of one of the polypeptides of the disclosure. Variant nucleic acid molecules also include synthetically derived nucleic acid molecules, such as those generated, for example, by using site-directed mutagenesis but which still encode a protein of the disclosure. Generally, variants of a particular nucleic acid molecule of the disclosure will have at least about 70%, 75%, 80%, 85%, 90%, 91%, 92%, 93%, 94%, 95%, 96%, 97%, 98%, 99% or more sequence identity to that particular polynucleotide as determined by sequence alignment programs and parameters as described elsewhere herein. Variants of a particular nucleic acid molecule of the disclosure (i.e., the reference DNA sequence) can also be evaluated by comparison of the percent sequence identity between the polypeptide encoded by a variant nucleic acid molecule and the polypeptide encoded by the reference nucleic acid molecule. Percent sequence identity between any two polypeptides can be calculated using sequence alignment programs and parameters described elsewhere herein. Where any given pair of nucleic acid molecule of the disclosure is evaluated by comparison of the percent sequence identity shared by the two polypeptides that they encode, the percent sequence identity between the two encoded polypeptides is at least about 70%, 75%, 80%, 85%, 90%, 91%, 92%, 93%, 94%, 95%, 96%, 97%, 98%, 99% or more sequence identity. In some embodiments, the term “variant” protein is intended to mean a protein derived from the native protein by deletion (so-called truncation) of one or more amino acids at the N-terminal and/or C-terminal end of the native protein; deletion and/or addition of one or more amino acids at one or more internal sites in the native protein; or substitution of one or more amino acids at one or more sites in the native protein. Variant proteins encompassed by the present disclosure are biologically active, that is they continue to possess the desired biological activity of the native protein as described herein. Such variants may result from, for example, genetic polymorphism or from human manipulation. Biologically active variants of a protein of the disclosure will have at least about 70%, 75%, 80%, 85%, 90%, 91%, 92%, 93%, 94%, 95%, 96%, 97%, 98%, 99% or more sequence identity to the amino acid sequence for the native protein as determined by sequence alignment programs and parameters described elsewhere herein. A biologically active variant of a protein of the disclosure may differ from that protein by as few as 1-15 amino acid residues, as few as 1-10, such as 6-10, as few as 5, as few as 4, 3, 2, or even 1 amino acid residue. The proteins or polypeptides of the disclosure may be altered in various ways including amino acid substitutions, deletions, truncations, and insertions. Methods for such manipulations are generally known in the art. For example, amino acid sequence variants and fragments of the proteins can be prepared by mutations in the nucleic acid sequence that encode the amino acid sequence recombinantly.

“Optional” or “optionally” means that the subsequently described event, circumstance, or material may or may not occur or be present, and that the description includes instances where the event, circumstance, or material occurs or is present and instances where it does not occur or is not present.

### Compositions and Systems

In one aspect, the disclosure relates to a composition comprising: (a) a first layer of cells comprising a plurality of cancer cells; (b) a second layer of cells comprising a plurality of stromal cells and extracellular matrix protein; wherein: (i) the density of the extracellular matrix protein is from about 60 to about 120 mg/mL; or (ii) the bulk elastic modulus across the first and second layer of cells is from about 6 KPa to about 10 KPa.

In some embodiments, the first layer of cells is positioned in a spheroid. In some embodiments, the first layer of cells is completely surrounded in three dimensions by the second layer of cells. In some embodiments, there is a “margin” area between the first layer of cells and the second layer of cells comprising a mixture of both cells from the first layer and cells from the second layer.

In some embodiments, the location where the first layer of cells and the second layer of cells are in contact is a tissue interface. In some embodiments, there is local anisotropy at the tissue interface. Anisotropy is a difference, when measured along different axes, in the physical or mechanical properties of the cells. Local anisotropy means that the principal stresses are unequal. In some embodiments, the second layer of cells are positioned around the cancer cells in an anistropic ring of cells.

In some embodiments, the cancer cells are carcinoma cells. In some embodiments, the cancer cells are breast cancer carcinoma cells. In some embodiments, the cancer cells are skin cancer carcinoma cells. In some embodiments, the cancer cells are prostate cancer carcinoma cells. In some embodiments, the cancer cells are lung cancer carcinoma cells. In some embodiments, the cancer cells are colon cancer carcinoma cells. In some embodiments, the cancer cells are sarcoma cells. The disclosure also relates to a composition comprising a vessel or cell reactor surface onto which a first and second layer of cells are positioned; the first layer comprising cancer cells and a second layer comprising stromal cells; wherein the interface between the first and second layers of cells comprises a dense stroma and gradients of increasing ECM density. In some embodiments, the ECM density comprises collagen I or collagen III density of from about 50 to about 150 mg/mL of volume of cells and tissue. In some embodiments, the ECM density comprises collagen I or collagen III density of from about 75 to about 150 mg/mL of volume of cells and tissue. In some embodiments, the ECM density comprises collagen I or collagen III density of from about 100 to about 150 mg/mL of volume of cells and tissue. In some embodiments, the cancer cells are carcinoma cells, the stromal cells are human fibroblasts and the ECM matric comprises or consists of Collagen I at an increasingly density gradient in the direction of the carcinoma cells.

In some embodiments, the stromal cells comprise or consist of fibroblasts. In some embodiments, the stromal cells comprise mesenchymal stem cells. In some embodiments, the stromal cells comprise pericytes. In some embodiments, the stromal cells comprise myofibroblasts. In some embodiments, the stromal cells comprise fibroblast-like stromal cells. In some embodiments, the stromal cells comprise tumor-associated stromal cells. In some embodiments, the stromal cells comprise cell surface markers such as CD44, CD29, CD45, CD105 and/or CD90. In some embodiments, the stromal cells are human fibroblasts.

In some embodiments, the stromal cells and the cancer cells are in culture for at least about 7 days. In some embodiments, the stromal cells and the cancer cells are in culture from about 7 days to about 24 days. In some embodiments the stromal cells and the cancer cells are in culture for at least about 14 days, at least about 21 days, at least about 28 days or more than 28 days.

In some embodiments, the first layer of cells is at least about 100 μm in diameter, at least about 200 μm in diameter, at least about 300 μm in diameter, at least about 400 μm in diameter, at least about 500 μm in diameter, at least about 600 μm in diameter, at least about 700 μm in diameter, at least about 800 μm in diameter, at least about 900 μm in diameter or at least about 1 mm in diameter.

In some embodiments, the second layer of cells is at least about 100 μm in thickness, at least about 200 μm in thickness, at least about 300 μm in thickness, at least about 400 μm in thickness, at least about 500 μm in thickness, at least about 600 μm in thickness, at least about 700 μm in thickness, at least about 800 μm in thickness, at least about 900 μm in thickness or at least about 1 mm in thickness.

In some embodiments, the extracellular matrix protein comprises Collagen I, Collagen III and/or fibronectin.

In some embodiments, the extracellular matrix is positioned within the second layer of cells. In some embodiments, the extracellular matrix is positioned around the plurality of cancer cells at a density from about 7 KPa to about 9 kPa.

In some embodiments, the density of the extracellular matrix protein is from about 60 to about 120 mg/mL. In some embodiments, the density of the extracellular matrix protein is from about 60 to about 110 mg/mL. In some embodiments, the density of the extracellular matrix protein is from about 60 to about 100 mg/mL. In some embodiments, the density of the extracellular matrix protein is from about 60 to about 90 mg/mL. In some embodiments, the density of the extracellular matrix protein is from about 60 to about 80 mg/mL. In some embodiments, the density of the extracellular matrix protein is from about 60 to about 70 mg/mL. In some embodiments, the density of the extracellular matrix protein is from about 70 to about 120 mg/mL. In some embodiments, the density of the extracellular matrix protein is from about 70 to about 110 mg/mL. In some embodiments, the density of the extracellular matrix protein is from about 70 to about 100 mg/mL. In some embodiments, the density of the extracellular matrix protein is from about 70 to about 90 mg/mL. In some embodiments, the density of the extracellular matrix protein is from about 70 to about 80 mg/mL. In some embodiments, the density of the extracellular matrix protein is from about 80 to about 120 mg/mL. In some embodiments, the density of the extracellular matrix protein is from about 80 to about 110 mg/mL. In some embodiments, the density of the extracellular matrix protein is from about 80 to about 100 mg/mL. In some embodiments, the density of the extracellular matrix protein is from about 80 to about 90 mg/mL. In some embodiments, the density of the extracellular matrix protein is from about 90 to about 120 mg/mL. In some embodiments, the density of the extracellular matrix protein is from about 90 to about 110 mg/mL. In some embodiments, the density of the extracellular matrix protein is from about 90 to about 100 mg/mL. In some embodiments, the density of the extracellular matrix protein is from about 100 to about 120 mg/mL. In some embodiments, the density of the extracellular matrix protein is from about 100 to about 110 mg/mL. In some embodiments, the density of the extracellular matrix protein is from about 110 to about 120 mg/mL.

In some embodiments, the extracellular matrix is in a densely packed layer with a thickness of about 10 μm to about 50 μm around the cancer cells relative to a position distal to the first layer of cells. In some embodiments the extracellular matrix is in a densely packed layer with a thickness of about 10 μm to about 40 μm around the cancer cells relative to a position proximate to the first layer of cells. In some embodiments the extracellular matrix is in a densely packed layer with a thickness of about 10 μm to about 30 μm around the cancer cells relative to a position proximate to the first layer of cells. In some embodiments the extracellular matrix is in a densely packed layer with a thickness of about 10 μm to about 20 μm around the cancer cells relative to a position proximate to the first layer of cells. In some embodiments the extracellular matrix is in a densely packed layer with a thickness of about 20 μm to about 50 μm around the cancer cells relative to a position proximate to the first layer of cells. In some embodiments the extracellular matrix is in a densely packed layer with a thickness of about 20 μm to about 40 μm around the cancer cells relative to a position proximate to the first layer of cells. In some embodiments the extracellular matrix is in a densely packed layer with a thickness of about 20 μm to about 30 μm around the cancer cells relative to a position proximate to the first layer of cells. In some embodiments the extracellular matrix is in a densely packed layer with a thickness of about 30 μm to about 50 μm around the cancer cells relative to a position proximate to the first layer of cells. In some embodiments the extracellular matrix is in a densely packed layer with a thickness of about 30 μm to about 40 μm around the cancer cells relative to a position proximate to the first layer of cells. In some embodiments the extracellular matrix is in a densely packed layer with a thickness of about 40 μm to about 50 μm around the cancer cells relative to a position proximate to the first layer of cells.

In some embodiments, the bulk elastic modulus across the first and second layer of cells is from about 6 KPa to about 10 KPa. In some embodiments, the bulk elastic modulus across the first and second layer of cells is from about 6 KPa to about 9 KPa. In some embodiments, the bulk elastic modulus across the first and second layer of cells is from about 6 KPa to about 8 KPa. In some embodiments, the bulk elastic modulus across the first and second layer of cells is from about 6 KPa to about 7 KPa. In some embodiments, the bulk elastic modulus across the first and second layer of cells is from about 7 KPa to about 10 KPa. In some embodiments, the bulk elastic modulus across the first and second layer of cells is from about 7 KPa to about 9 KPa. In some embodiments, the bulk elastic modulus across the first and second layer of cells is from about 7 KPa to about 8 KPa. In some embodiments, the bulk elastic modulus across the first and second layer of cells is from about 8 KPa to about 10 KPa. In some embodiments, the bulk elastic modulus across the first and second layer of cells is from about 8 KPa to about 9 KPa. In some embodiments, the bulk elastic modulus across the first and second layer of cells is from about 9 KPa to about 10 KPa.

Bulk elastic modulus is the ratio of pressure applied to the corresponding relative decrease in the volume of the material. It is represented by the formula:

$$B = Δ P / ( Δ V / V ) ,$$

wherein
B=Bulk Modulus
ΔP=change of the pressure or force applied per unit area on the material
ΔV=change of the volume of the material due to the compression
V=Initial volume of the material
   Bulk elastic modulus can be measured by any method known in the art.

In some embodiments, the composition further comprises a hydrogel.

In some embodiments, the hydrogel is absorbed or immobilized to a solid substrate or solid support. The terms solid substrate or solid support can be used interchangeably and refers to any substance that is free or substantially free of cellular toxins. In some embodiments, the solid substrate comprise one or a combination of silica, plastic, and metal. In some embodiments, a system comprising a cell culture unit is utilized to culture and expand cancer cells described herein, in the presence or absence of stromal cells. In some embodiments, the cell culture unit comprises one or a plurality of cell reactor surfaces housed in at least a first compartment, the one or plurality of cell reactor surfaces in fluid connection with a first and second media line, the first media line in fluid communication with a first media inlet, the second media line in fluid communication to a first media outlet. In some embodiments, the one or plurality of cell reactor surfaces are configured in a cylindrical form with a hollow volume fixed within a cylindrical first compartment; wherein the first media line and the second media line are positioned on opposite faces of the cylindrical first compartment. The first media line can be attached to a first sealable aperture configured for sterile attachment of a cell culture media source. In some embodiments, the system further comprises a pump and a fluid regulator in operable contact with the first media line, wherein the pump is capable of generating pressure in the first media line and wherein the fluid regulator is capable of regulating a rate of fluid (such as tissue culture media) from a position in a fluid circuit to the pump through the first compartment and into the second media line.

In some embodiments, a system comprising a cell culture unit is utilized to culture and expand cancer cells described herein, in the presence or absence of stromal cells. In some embodiments, the cell culture unit comprises one or a plurality of cell reactor surfaces housed in at least a first compartment, the one or plurality of cell reactor surfaces in fluid connection with a first and second media line, the first media line in fluid communication with a first media inlet, the second media line in fluid communication to a first media outlet. In some embodiments, the one or plurality of cell reactor surfaces are configured in a cylindrical form with a hollow volume fixed within a cylindrical first compartment; wherein the first media line and the second media line are positioned on opposite faces of the cylindrical first compartment. The first media line can be attached to a first sealable aperture configured for sterile attachment of a cell culture media source. In some embodiments, the system further comprises a pump and a fluid regulator in operable contact with the first media line, wherein the pump is capable of generating pressure in the first media line and wherein the fluid regulator is capable of regulating a rate of fluid (such as tissue culture media) from a position in a fluid circuit to the pump through the first compartment and into the second media line.

The one or plurality of cell reactor surfaces can have a surface area from about 0.5 m² to about 100.0 m², including any value therein, such as about 3 m², about 4 m², about 5 m², about 6 m², about 7 m², about 8 m², about 9 m², about 10 m², about 11 m2, about 12 m², about 13 m², about 14 m², about 15 m², about 16 m², about 17 m², about 18 m², about 19 m², about 20 m², about 21 m², about 22 m², about 23 m², about 24 m², about 25 m², about 26 m², about 27 m², about 28 m², about 29 m², about 30 m², about 31 m², about 32 m², about 33 m2, about 34 m2, about 35 m2, about 36 m², about 37 m2, about 38 m², about 39 m2, about 40 m², about 41 m², about 42 m², about 43 m², about 44 m², about 45 m², about 46 m², about 47 m², about 48 m², about 49 m², about 50 m², about 51 m², about 52 m², about 53 m², about 54 m², about 55 m², about 56 m², about 57 m², about 58 m², about 59 m², about 60 m², about 61 m², about 62 m², about 63 m², about 64 m², about 65 m², about 66 m², about 67 m², about 68 m², about 69 m², about 70 m², about 71 m², about 72 m², about 73 m², about 74 m², about 75 m², about 76 m², about 77 m², about 78 m², about 79 m², about 80 m², about 81 m², about 82 m², about 83 m², about 84 m², about 85 m², about 86 m², about 87 m², about 88 m², about 89 m², about 90 m², about 91 m², about 92 m², about 93 m², about 94 m², about 95 m², about 96 m², about 97 m2, about 98 m², or about 99 m2, or about 100 m2, or about 105 m2.

The system further comprises a gas transfer module in operable connection to the one or plurality of cell reactor surfaces. In some embodiments, the gas module comprises a gas pump and a gas regulator connected to the first compartment by a first gas line. In such embodiments, the first compartment comprises at least one gas outlet. The gas pump is capable of generating air pressure from the pump to the first compartment through the first gas line. The gas outlet can be one or more vents or the gas outlet can be configured for sterile connection to one or more vents. The gas regulator is capable of regulating the speed of gas from the pump through the first compartment.

Some embodiments further comprise a first gas inlet in operable connection to the gas transfer module. In some embodiments, the first gas inlet is attached to a second sealable aperture configured for sterile attachment of a gas source. The gas source can be any known gas storage and/or delivery system, such as for example a container or a tank.

The system can further comprise an apheresis unit in fluid communication with the cell culture unit. Suitable apheresis units include the Spectra Optia Apheresis System (TerumoBCT).

Additionally, in some embodiments, the system further comprises a harvesting compartment in fluid communication with the cell culture unit. Suitable harvesting compartments are discussed elsewhere herein.

A cell culture system as described herein can be used to cancer cells through culturing one or a plurality of stromal cells in the system and allowing the cancer cells and the stromal cells to grow in the first compartment for a time period sufficient to proliferate. In some embodiments, the time period is sufficient to allow the stromal cells to exhibit compaction and grow to a disclosed density around the cancer cells. Cells of the disclosure can be initially introduced into the system and seeded on to the cell reactor surface through on opening to the system's first compartment. After seeding the cells, tissue culture media may be pumped into the system through a fluid circuit that is an open or closed fluid circuit.

The disclosure also relates to a system comprising a cell culture unit comprising one or a plurality of cell reactor surfaces housed in a plurality of compartments, each compartment separated by a removable partition first compartment comprising at least one cell reactor surface, at least one cell reactor surface in fluid connection with a first and second media line, the first media line in fluid communication with a first media inlet, the second media line in fluid communication to a first media outlet. In some embodiments, the cell culture unit comprises a single cell culture chamber comprising multiple partitions, each partition independently removable and independently in fluid connection with the first and the second media line and each partition or set of partitions defining a distinct compartment. In some embodiments, the cell culture unity comprises 2, 3, 4, 5, 6, 7, 8, 9, 10, 11, 12 or more compartments, each compartment separated by and/or defined by one or more partitions. In some embodiments, the compartments are configured in a grid or linear pattern. In some embodiments, each partition separating one compartment from another compartment may be removed such that the cell reactor surface of a first compartment is or becomes contiguous with a cell reactor surface of a second compartment. The removal of one or more partitions allows for an increased surface area onto which cells from one compartment (such as the first compartment) may proliferate and/or grow into another compartment (such as the second compartment) during a method of culturing. In some embodiments, the cell culture unit comprises a set of side walls defining a single surface area divided among 2, 3, 4, 5, 6, 7, 8, 9, 10, 11, 12 or more compartments each compartment with at least one or a plurality of cell reactor surfaces. In some embodiments, each compartment has at least a first cell reactor surface. The disclosure relates to a method of growing T-cell populations on a tissue culture system disclosed herein, wherein primary sets of lymphocytes are plated at about a concentration of from about 0.001 to about 10 million cells per milliliter into one or more compartments of the cell culture unit and then allowed to grow to a confluent layer on surface area of from about 1 to about 200 squared centimeters. In some embodiments, the method further comprises removing one or more partitions to allow the cells to grow in a second compartment until confluence, when again, optionally, another partition may successively be removed to allow for more surface are for expanded culture. In some embodiments the method of culturing further comprises repeating the step of removing a partition for each of the compartments into which cells should grow. In some embodiments, the cell culture unit comprises 3, 4, 5, 6, 7, 8, 9, 10, 11, 12 or more partitions each of which corresponding to the physical barrier between a second and third compartment, between a third and fourth compartment, between a fourth and fifth compartment, between a fifth and sixth compartment, between a sixth and seventh compartment, between a seventh and eighth compartment, between an eighth and ninth compartment, between a ninth and tenth compartment, between a tenth and eleventh compartment, and/or between an eleventh and twelfth compartment, respectively.

In some embodiments, one or more of the partitions comprise an interior portion, a frame portion and an exterior portion. The interior portion of the partition is positioned in the closed portion of the system; the frame portion spans a wall of the culture system separating the interior of the culture system to the exterior of the system; and the exterior portion is positioned outside of the system. In some embodiments, a seal operably fits around the frame portion of one or more of the partitions such that removal of the partition does not introduce pathogens to and/or does not expose the environment outside of the tissue culture system to the interior of the tissue culture system.

In some embodiments, the cell density of each compartment is from about 0.1 to about 10 million cells per mL of cell culture media. In some embodiments, the cell density of each compartment is from about 0.1 to about 10 million cells per mL of cell culture media. In some embodiments, the cell density of each compartment is from about 0.5 to about 10 million cells per mL of cell culture media. In some embodiments, the cell density of each compartment is from about 1.0 to about 10 million cells per mL of cell culture media. In some embodiments, the cell density of each compartment is from about 2 to about 10 million cells per mL of cell culture media. In some embodiments, the cell density of each compartment is from about 3 to about 10 million cells per mL of cell culture media. In some embodiments, the cell density of each compartment is from about 4 to about 10 million cells per mL of cell culture media. In some embodiments, the cell density of each compartment is from about 5 to about 10 million cells per mL of cell culture media. In some embodiments, the cell density of each compartment is from about 6 to about 10 million cells per mL of cell culture media. In some embodiments, the cell density of each compartment is from about 7 to about 10 million cells per mL of cell culture media. In some embodiments, the cell density of each compartment is from about 8 to about 10 million cells per mL of cell culture media. In some embodiments, the cell density of each compartment is from about 9 to about 10 million cells per mL of cell culture media. In some embodiments, the cell density of each compartment is from about 0.1 to about 20 million cells per mL of cell culture media. In some embodiments, the cell density of each compartment is from about 0.1 to about 50 million cells per mL of cell culture media.

In some embodiments, the stromal cells are at a density from about 400,000 cells per mL of volume to about 1,550,000 cells per mL of volume. In some embodiments, the stromal cells are at a density from about 400,000 cells per mL of volume to about 1,500,000 cells per mL of volume. In some embodiments, the stromal cells are at a density from about 400,000 cells per mL of volume to about 1,000,000 cells per mL of volume. In some embodiments, the stromal cells are at a density from about 400,000 cells per mL of volume to about 750,000 cells per mL of volume. In some embodiments, the stromal cells are at a density from about 400,000 cells per mL of volume to about 500,000 cells per mL of volume. In some embodiments, the stromal cells are at a density from about 500,000 cells per mL of volume to about 1,500,000 cells per mL of volume. In some embodiments, the stromal cells are at a density from about 500,000 cells per mL of volume to about 1,250,000 cells per mL of volume. In some embodiments, the stromal cells are at a density from about 500,000 cells per mL of volume to about 1,000,000 cells per mL of volume. In some embodiments, the stromal cells are at a density from about 500,000 cells per mL of volume to about 750,000 cells per mL of volume. In some embodiments, the stromal cells are at a density from about 750,000 cells per mL of volume to about 1,500,000 cells per mL of volume. In some embodiments, the stromal cells are at a density from about 750,000 cells per mL of volume to about 1,250,000 cells per mL of volume. In some embodiments, the stromal cells are at a density from about 750,000 cells per mL of volume to about 1,000,000 cells per mL of volume. In some embodiments, the stromal cells are at a density from about 1,000,000 cells per mL of volume to about 1,500,000 cells per mL of volume. In some embodiments, the stromal cells are at a density from about 1,250,000 cells per mL of volume to about 1,500,000 cells per mL of volume.

In some embodiments, the plurality of fibroblasts are free of one or more contact points that exert tension on the plurality of cancer cells.

In some embodiments, tension exerted on the plurality of cancer cells by the stromal cells is not modulated by contact of the plurality of stromal cells to a synthetic element that creates point of tension exacted on the stromal cells.

The disclosure is also directed to a composition comprising (a) a plurality of stromal cells; (b) a plurality of cancer cells; (c) extracellular matrix protein; and wherein the plurality of cancer cells are in a three-dimensional shape and define a first layer of cells; wherein the plurality of stromal cells are positioned around the cancer cells [in densely packed concentric rings of cells] in a second layer of cells; and wherein the extracellular matrix protein is positioned within the second layer of cells.

### Methods

In some embodiments, the system further comprises one or combination of culture mediums disclosed herein.

The disclosure also relates to a method of assaying affect or toxicity of an agent relative to a cancer cell or tumor in vitro, comprising contacting an agent to a layer of stromal cells or a layer of cancer cells disclosed herein.

In some embodiments, the system further comprises one or combination of culture mediums disclosed herein. The disclosure also relates to a method of assaying affect or toxicity of an agent relative to a cancer cell or tumor in vitro, comprising contacting an agent to a layer of stromal cells or a layer of cancer cells disclosed herein. In some embodiments, the method further comprises exposing the one or more cancer cells to an agent. In some embodiments, measuring the one or more morphometric changes comprises measuring morphometry of the one or more cancer cells. The present disclosure also relates to a method of evaluating the toxicity of an agent comprising: (a) culturing one or more cancer cells in any of the compositions described herein; (b) exposing at least one agent to the one or more cancer cells; (c) measuring and/or observing one or more morphometric changes of the one or more cancer cells; and (d) correlating one or more morphometric parameters of the one or more cancer cells with the toxicity of the agent, such that, if the morphometric parameters are indicative of decreased cell viability, the agent is characterized as toxic and, if the morphometric parameters are indicative of unchanged or positive cell viability, the agent is characterized as non-toxic.

In some embodiments, measuring the one or more morphometric changes comprises measuring the compound action potential of the one or more cancer cells.

The present disclosure also relates to a method of inducing growth of one or a plurality of cancer cells in a three dimensional culture vessel comprising a solid substrate, said method comprising: (a) contacting one or a plurality of isolated cancer cells with the solid substrate, said substrate comprising at least one exterior surface, at least one interior surface and at least one interior chamber defined by the at least one interior surface and accessible from a point exterior to the solid substrate through at least one opening; (b) seeding one a plurality of isolated stromal cells to the at least one interior chamber; (c) applying a cell medium into the culture vessel with a volume of cell medium sufficient to cover the at least one interior chamber; wherein the interior chamber comprises a hydrogel. In some embodiments, the stromal cells are fibroblasts, myofibroblasts, mesenchymal cells, or bone cells. In some embodiments, the stromal cells are a single type of stromal cell seeded with at least about 400 thousand, about 500 thousand, about 600 thousand cells. In some embodiments, the stromal cells are a single type of stromal cell seeded with at least about 400 thousand, about 500 thousand, about 600 thousand cells or more and then allowed to be in culture for a time period sufficient for a highly dense ECM protein to form at the interface between the first and second layer of cells a disclosed herein.

In some embodiments, the method further comprises a one or plurality of cancer cells with at least one agent. In some embodiments, the at least one agent is one or a plurality of stem cells or modified T cells. In some embodiments, the T-cells are CAR T cells.

In some embodiments, the at least one agent comprises one or a combination of: laminin, insulin, transferrin, selenium, BSA, FBS, ascorbic acid, type I collagen, and type III collagen. In some embodiments, the at least one agent comprises a small chemical compound. In some embodiments, the at least one agent comprises at least one environmental pollutant. In some embodiments, the at least one agent comprises one or a combination of small chemical compounds chosen from: chemotherapeutics, analgesics, cardiovascular modulators, cholesterol, neuroprotectants, neuromodulators, immunomodulators, anti-inflammatories, and anti-microbial drugs.

The present disclosure also relates to a method of detecting and/or quantifying cancer cell growth comprising: (a) quantifying one or a plurality of cancer cells; (b) culturing the one or more cancer cells in any of the compositions disclosed herein; and (c) calculating the number of cancer cells in the composition after a culturing for a time period sufficient to allow growth of the one or plurality of cells.

In some embodiments, step (c) comprises detecting an internal and/or external recording of such one or more cancer cells after culturing one or more cancer cells and correlating the recording with a measurement of the same recording corresponding to a known or control number of cells. In some embodiments, the method further comprises contacting the one or more cancer cells to one or more agents. In some embodiments, step (c) comprises measuring an internal and/or external recording before and after the step of contacting the one or more cancer cells to the one or more agents; and correlating the difference in the recording before contacting the one or more cancer cells to the one or more agents to the recording after contacting the one or more cancer cells to the one or more agents to a change in cell number.

The present disclosure also relates to a method of detecting or quantifying of cancer cell growth comprising: (a) quantifying the one or plurality of cancer cells in one or more of the composition disclosed herein; (b) contacting the one or plurality of cancer cells to one or a plurality of agents; and (c) quantifying the amount of biomarker expression in the one or plurality of cells after contacting the one or plurality of cells to one or a plurality of agents; and (d) calculating the difference in the number of cancer cells in culture prior to the step (c) and after step (c). The present disclosure also relates to a method of detecting or quantifying of cancer cell growth comprising: (a) seeding one or a plurality of cancer cells in any of the compositions disclosed herein; (b) quantifying expression of one or more biomarkers in the one or plurality of cancer cells; (c) contacting the one or plurality of cancer cells to one or a plurality of agents; and (d) quantifying one or more biomarkers in the one or plurality of cells after contacting the one or plurality of cells to one or a plurality of agents; and (e) calculating the difference in the number of cancer cells in culture prior to the step (c) and after step (c). In some embodiments, the step of quantifying comprises staining the one or plurality of a cancer cells. In some embodiments, steps (b), (d), and/or (e) are performed via microscopy or digital imaging.

The present disclosure also relates to a method of measuring intracellular or extracellular expression of nucleic acid (e.g. mRNA expression) or expression of a protein biomarker comprising: (a) culturing one or a plurality of cancer cells in any of the composition disclosed herein; (b) allowing the one or a plurality of cancer cells to form a spheroid (c) mixing the cells with stromal cells and ECM protein or proteins; (d) seeding the cells in a vessel or on cell reactor surface; (e) allowing the stromal cells and ECM protein or proteins to form dense anisotropic tissue around at least a portion or around the entire spheroid; and (f) taking a sample of the stromal cells or the cancer cells; (g) isolating RNA or protein from the sample; and (h) measuring the quantity of biomarkers from the RNA or protein sample. In some embodiments, the step of measuring the quantity of biomarkers comprises conducting RT-PCR or performing immunohistochemistry. In some embodiments, the stromal cells are human fibroblasts and the cancer cells are human carcinoma cells. In some embodiments, the method further comprises exposing the stromal cells and cancer cells to an agent after step (e). In some embodiments, the method further comprises exposing the stromal cells and cancer cells to an agent after step (e); and measuring the quantity of biomarkers from the RNA or protein sample before and after exposing step.

The present disclosure also relates to a method of measuring or quantifying any therapeutic effect of an agent comprising: (a) culturing one or a plurality of cancer cells in any of the composition disclosed herein in the presence and absence of the agent; (b) applying a voltage potential across the one or a plurality of cancer cells in the presence and absence of the agent; or measuring the modulus or tension on the one or plurality of cancer cells and/or stromal cells in the presence and absence of the agent or observing the morphological changes of the stromal cells or cancer cells; and (c) correlating the difference in tension or modulus or change of morphology through the one or plurality of cancer cells to the therapeutic effect of the agent, such that a decline in viability or cell health or a decrease in tension in the presence of the agent as compared to the tension measured or morphology observed in the absence of the agent is indicative of a therapeutic effect and no change or an increased tension or no change in morphology or viability in the presence of the agent as compared to the same measured or observed in the absence of the agent is indicative of the agent not conferring a therapeutic effect.

The present disclosure also relates to a method of detecting or quantifying morphology changes due to the presence, absence or change in the amount of agent exposed to the cells in vitro comprising: (a) culturing one or a plurality of cancer cells and fibroblasts with ECM protein in any of the composition disclosed herein; (b) exposing an agent to the cells; and (c) measuring or observing an effect of the agent on the one or plurality of cancer cells. In some embodiments, the method further comprises correlating a reduced viability of the cancer cells in step (c) to the positive effect of the agent as compared to the same measurements or observations of the cancer cells and stromal cells not exposed to the agent. In some embodiments, the method further comprises observing the cells through imaging the one or plurality of cells with a microscope and/or digital camera.

The present disclosure also relates to a method of culturing a carcinoma cell in culture comprising: (a) culturing one or a plurality of carcinoma cells in any of the composition disclosed herein; and (b) exposing the cancer cells to an agent in the presence of a layer of stromal cells and ECM material. In some embodiments, the interface between a plurality of carcinoma cells and fibroblasts comprise a gradient of increasing density of ECM material or protein (such as Collagen I and Collagen III) in the direction proximate to the cancer cells. In some embodiments, the methods are free of exertion of tension on the carcinoma cells except the tension created by the second layer of cells (stromal cells).

The present disclosure also relates to a method of measuring or quantifying toxicity of an agent comprising: (a) culturing one or a plurality of cancer cells in any of the composition disclosed herein in the presence and absence of the agent; (b) measuring the Young's or bulk modulus or tension across the one or plurality of cancer cells and/or stromal cells in the presence and absence of the agent or observing the morphological changes of the stromal cells or cancer cells; and (c) correlating the difference in tension or modulus or change of morphology through the one or plurality of cancer cells to toxicity of the agent, such that a decline in viability or cell health or a decrease in tension in the presence of the agent as compared to the tension measured or morphology observed in the absence of the agent is indicative of a toxic effect and no change or an increased tension or no change in morphology or viability in the presence of the agent as compared to the same measured or observed in the absence of the agent is indicative of the agent not conferring a toxic effect.

Other embodiments are described in the following non-limiting Examples. Various publications, including patents, published applications, technical articles and scholarly articles are cited throughout the specification. Each of these cited publications is incorporated by reference herein in its entirety.

### EXAMPLE

### Example 1

A commercially available stereolithography (SLA) printer (F3, Formlabs) was characterized and optimized for manufacturing resin molds suitable for MFOC fabrication using standard polydimethylsiloxane (PDMS) soft lithography and PDMS stamping for multilayer bonding. Printer resolution limits for positive and negative channel and pillar features were determined.

Workflows were established for: i) removing volatile components trapped in printed resins to enable complete PDMS curing in resin molds; ii) flattening of warped resin molds to produce flat PDMS devices; iii) urethane clear coating of resin molds to produce transparent PDMS devices.

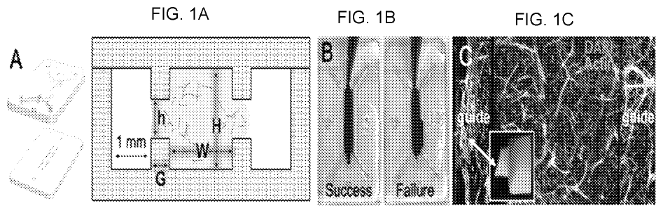

An array of designs varying membrane free organ chip (MFOC) dimensions (FIG. 1) were tested in single user tests performing 10 injections per design. Key dimensions varied included tissue lane height (H) and width (W), and the height of membrane-free side channel interface (h), which determines the important design parameter h/H. With MFOC designs that enabled up to 100% liquid pinning rates, tissue vascularization in MFOC was tested by loading human lung fibroblasts (HLFs) and human umbilical vein endothelial cells (HUVECs) in a collagen and fibrin hydrogel blend and culturing for 5 days. Our design enabled up to 100% liquid pinning success rates and rapid formation of milliscale vascularized tissues.

Emphasizing engineering reduction to practice during the MPS design phase will accelerate non-expert adoption. Ongoing work includes adopting the design for fully vascularized multi-organ MPS.

Our design enabled high liquid pinning success rates and rapid formation of milliscale vascularized tissues. Emphasizing engineering reduction to practice during the MPS design phase will accelerate non-expert adoption.

A design-test-iterate approach was applied to optimize MFOC geometry and achieve reduction to practice, which we defined as 100% success rate loading devices (no blowout failure) and achieving milliscale tissue vascularization in as little as 5 days.

In some studies, we designed, tested, and validated a prototype 4-organ chip that enables user-friendly injection of hydrogels containing mixtures of cells and/or spheroids/organoids.

Devices were 38×38 mm with an overall thickness of 2 mm. The tissue lanes of each organ module were 1.25 wide, 14.235 mm long, and 1 mm high. Our milliscale implementation required incorporation of top and bottom guide structures (0.285 mm wide, 0.25 mm high) that separate the central tissue lanes and the flanking fluidic channels. For culture experiments, devices were fit with a cap layer that created fluid reservoirs above each channel port.

Vascularization of each organ module was tested by loading human lung fibroblasts (HLFs) and human umbilical vein endothelial cells (HUVECs) (2×106/ml of each cell type) in a collagen and fibrin hydrogel blend and culturing in static for up to 9 days. Tissues were fixed with 4% paraformaldehyde and processed for in-device staining and laser scanning confocal microscopy. Endothelial cells were labeled with Dylight594-conjugated Ulex Europeas Agglutinin-1. Actin cytoskeleton was stained with Alexa488-conjugated phalloidin.

The dimensions of each individual organ module in our 4-organ device was optimized based on parallel studies in single organ devices focused on optimizing the success rate of injections in the tissue lane for each organ compartment.

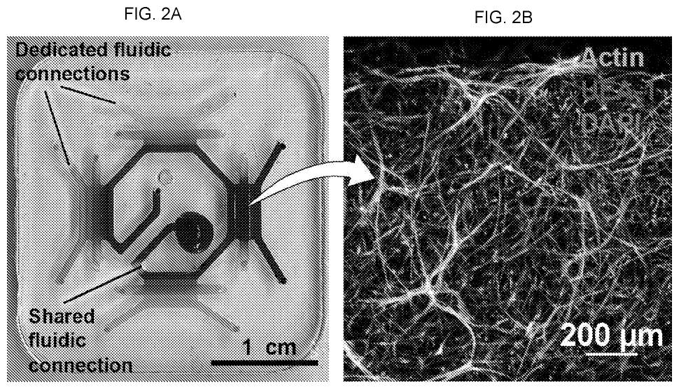

A loading forgiveness test using nine separate 4-organ devices resulted in a 97% success rate (35 out of 36 successful injections). We used a combination of food dye colors to visualize the compartmentalization of our devices, which feature a dedicated fluidic connection for each organ module and a shared fluidic connection for factor exchange between organ modules (FIG. 2). This design will help address the issue of universal culture medium vs. specific medium formulations.

A universal medium can be used in the shared connection, while organ-specific medium can be introduced in the dedicated connection for each module. The milliscale size of our design, and ease of device assembly by peeling apart the stamped layers, will allow users to harvest sufficient tissue volumes for protein extraction and tissue homogenization-based assays, such as enzyme activity assays. Culture tests in the device demonstrated rapid formation of vasculature spanning the entire 1.2 mm tissue (FIG. 2).

For model development, rapid tissue vascularization was achieved by loading human lung fibroblasts (HLFs) and human umbilical vein endothelial cells (HUVECs) in a collagen and fibrin hydrogel blend and seeding of the gel walls with endothelial cells.

Homogenization of patient-derived tumor tissues is accomplished is less than 1 hour by mechanical and enzymatic dissociation with a cocktail of collagenase, dispase, and DNase, followed by filtering through a 250 μm mesh to yield a preparations of tissue fragments.

We engineered TNBC tissue density in MFOC by varying the volume ratio of tumor homogenate and collagen-fibrin gel. TNBC tissue viability and proliferation in MFOC was assessed by Live/Dead and Ki67 staining, respectively. When tumors were unavailable, spheroids of patient-derived TNBC cell lines were used to optimize formation of a perfusable vascularized model.

Vascular network morphogenesis and anastomosis were visualized by 3D confocal imaging of tissues stained with endothelial-specific lectins. Anastomosis and network patency were further confirmed by FITC-dextran perfusion.

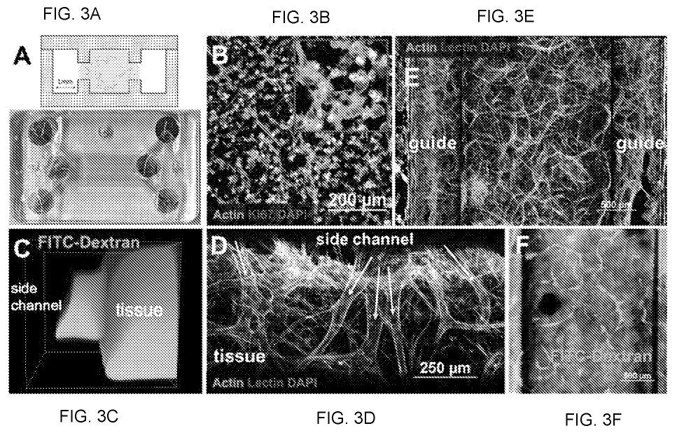

A novel milliscale MFOC design with multiple guide structures enabled 100% liquid pinning success rates of volumes up to 1.5×2×12 mm (FIG. 3). This capability enables rapid production of vascularized tissues with volumes up to 36 μm³; an order of magnitude greater than tissue volumes in microscale membrane-free systems.

Platforms that allow for tumor growth in vitro will be required to replace xenograft models. Our current tumor dissociation and processing protocol yields partially-digested injectable homogenates that form dense, viable TNBC tissues with large fractions of Ki67-positive proliferating cells in short-term MFOC culture (FIG. 3).

We characterized the tissue boundary geometry in MFOC and established a method for rapid tissue wall endothelialization and complete vascular network anastomosis (FIG. 3) that does not require chemical or fluid flow gradients which are difficult to implement and standardize.

Our MFOC design enables rapid production of fully vascularized milliscale TNBC explant tissues that will enable preservation and expansion of this precious clinical resource and simultaneous investigation of potential biological underpinnings of the TNBC health disparity.

A stereolithography (SLA) printer and customized post-processing workflow were used to manufacture molds for polydimethylsiloxane (PDMS) soft lithography. Molded PDMS layers were bonded by PMDS stamping with partitioning polyester membranes to fabricate fully-assembled devices.

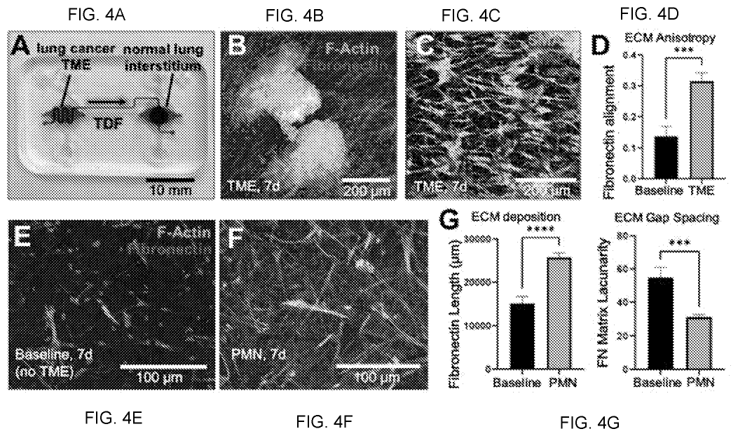

For our TME compartment (FIG. 3), lung adenocarcinoma (A549) cell spheroids seeded at a density of 1000 cells/spheroid were grown for 5 days and each TME chamber was loaded with 6 spheroids in collagen gel (3.5 mg/ml) with 3×10⁵/ml patient-derived cancer associated fibroblasts (CAF). Normal human lung fibroblasts (HLF) were subjected to a 14-day serum reduction in 2D that enables a quiescence-like state in 3D culture to study activation. The normal lung compartment (FIG. 4), was loaded with 3×10⁵/ml serum-reduced HLF in collagen gel.

Control devices contained serum-reduced HLF only in both compartments. All device cultures were maintained with fibroblast growth medium containing 0.1% FBS and 1% ITS. For endpoint analysis, we used whole mount immunofluorescent staining for the stromal activation markers SMA and activated YAP1 and the ECM protein fibronectin (FN), confocal microscopy, and a suite of 3D image analysis metrics provided by the FIJI macro TWOMBLI to quantify malignant characteristics of the TME compartment and pathological changes reflective of PMN cultivation.

The TME compartment in our devices exhibited features of aggressive cancers after 7 days of culture including 3D invasion of tumor cells and formation of a stroma comprised of greater than 90% activated CAF (SMA and aYAP1 positive) and a dense anisotropic matrix of newly-synthesized fibronectin (FN).

TDF transported from the lung cancer TME to the normal lung interstitium over 7 days elicited a stromal reaction indicative of PMN cultivation, as evidenced by markedly increased focal deposition of fibronectin, increased total fibronectin fiber length and decreased spacing of the fibronectin matrix. Thus, we have captured cancer-induced changes in the density and architecture of newly-synthesized ECM in a distant tissue that would support tumor cell adhesion.

### Example 2. Adaptable Benchtop Manufacturing of Milliscale Vascularized Microphysiological Systems

The resolution of low-cost stereolithography (SLA) printers now infuses organ chip design and fabrication with benchtop rapid prototyping capabilities. However, geometric distortion and surface roughness of molds fabricated using SLA resins impede effective organ chip manufacturing. Herein, post-processing procedures are described to manufacture SLA-printed molds that produce flat and fully cured PDMS parts. An optional polyurethane mold coating step enables tuning of the optical clarity and hydrophobicity of molded PDMS surfaces. This workflow allows low-cost rapid prototyping of vertically layered, membrane-bound organ chips and horizontally layered, membrane-free organ chips (MFOC) to engineer microphysiological systems (MPS) in non-expert laboratories. Optimization of MFOC performance is achieved via user injection loading tests with iterated designs. Morphometric analysis of milliscale bulk tissue vasculogenesis in MFOC is used to gauge network maturation as a foundation for modeling angiogenic processes and vascular inflammation. The core MFOC design is easily expanded to create a model of cell migration in contiguous adjacent tissue layers and a multi-tissue model of cancer-induced vascular inflammation constructed using a device with four fluidically interconnected MFOC compartments. An MFOC seeding and culture protocol is described that enables the rapid production of perfusable and patent vasculature spanning milliscale tissues in less than 10 days.

Early organ chips were constructed using methods developed to fabricate microfluidic devices which relied on photolithography to create silicon master molds used for downstream polydimethylsiloxane (PDMS) soft lithography.¹ Advances in digital manufacturing now enable low-cost, rapid prototyping capabilities that eliminate the need for cleanroom access and cut the mold fabrication time from days to hours.² Commercial stereolithography (SLA) based 3D printers deliver the resolution and accuracy necessary to fabricate master molds for PDMS soft lithography.²⁻⁴ Lower cost and faster manufacturing of SLA printed parts comes with the tradeoffs of material property limitations. Parts printed using low-cost SLA resins are prone to warping upon drying and curing and have been shown to leach chemicals that can inhibit PDMS curing and injure cells.^{5,6} Furthermore, the surface roughness of SLA-printed molds negates the accurate feature replication, ease of release, and transparency of molded PDMS that are hallmarks of silicon master molds used to fabricate microfluidic devices.⁷ We developed widely adaptable low-cost procedures that correct these limitations and enable efficient application-specific organ chip manufacturing in non-expert laboratories, as needed to accelerate the proliferation of MPS technology in preclinical biomedical science.

First-generation organ chip designs still commonly used in academia and industry utilize stacked PDMS channel layers separated by porous membranes (vertical organ chips).¹ Porous membranes are traversable by some cell types but disrupt the natural continuum between tissue layers.⁸ Researchers developed horizontally configured organ chips that utilize surface tension created by various barrier feature designs to pattern adjacent regions of hydrogel tissue constructs and liquid media channels via surface tension without the need for a continuous partitioning membrane (termed membrane-free organ chips in this paper). Early membrane-free organ chip (MFOC) designs featured pillars that trap injected liquids in patterned regions of a culture device.⁹ Later membrane-free designs featured rectangular rail structures, commonly referred to as ‘phase guides’, which use surface tension to pattern liquids in microfluidic devices.^{10,11} We scaled up to millimeter dimensions to work within the accurate resolution range of our SLA printer while aiming to produce devices for modeling bulk processes involving tissue structures spanning more than one millimeter. Breaching of injected liquids beyond patterned regions established by designed guide structures is the primary MFOC failure mode. We describe how MFOC design features such as PDMS surface properties, guide structure dimensions, and bulk tissue volume impact this failure mode and report user testing procedures for achieving MFOC reduction to practice in any laboratory setting.

We describe the use of MFOC for modeling tissue scale processes in vascular physiology and pathophysiology. We first established a workflow of morphometric analysis to quantify network maturation during milliscale bulk tissue vasculogenesis. We then utilized the tissue patterning capabilities of MFOC to engineer a model of cell migration between contiguous adjacent tissue layers with separately definable compositions. We demonstrate that the sequence of hydrogel injection and polymerization influences the properties of the interface between adjacent layers. This simple modification of the loading procedure grants the ability to engineer interfaces with no discernable border between layers or create an interfacial layer of dense ECM that impedes cell migration. The adjacent tissue layer model has the potential for wide application in modeling the influence of ECM composition and density at tissue interfaces on tissue scale processes such as collective cell migration, angiogenesis, and cancer invasion.

We leveraged scalability and modularity of the MFOC design used to establish bulk tissue vasculogenesis by connecting four MFOC modules in series with a shared communication channel and dedicated feeding channels for each module to create a highly-configurable prototype multi-organ system. We used the four MFOC design to model propagation of cancer-induced vascular inflammation in local and distant tissue compartments and successfully demonstrated the ability to engineer distant-dependent gradients of inflammatory activation. These data demonstrate the ability to model dynamic processes of cancer pathophysiology such as premetastatic niche cultivation in easily constructed MPS. User-defined iterations of this multi-organ platform have the potential to accelerate hypothesis testing for anti-metastasis treatment paradigms and facilitate the discovery of biomarkers for advanced cancer processes in human relevant systems.

We present a simple two-step MFOC seeding protocol to engineer milliscale tissues with a perfusable and patent internal vasculature in the absence of externally applied flows or growth factor gradients. Collectively, we present manufacturing and tissue engineering procedures that will enable researchers in a wide range of laboratory settings to create organ chip models of physiological and disease processes involving the interaction of multiple tissue compartments. These models can occupy a valuable niche for target validation between higher throughput but less complex in vitro screening platforms and animal models that capture whole organism physiology but often display incongruence with human disease and treatment responses.

### Milliscale Organ Chip Manufacturing Using Low-Cost SLA Printers

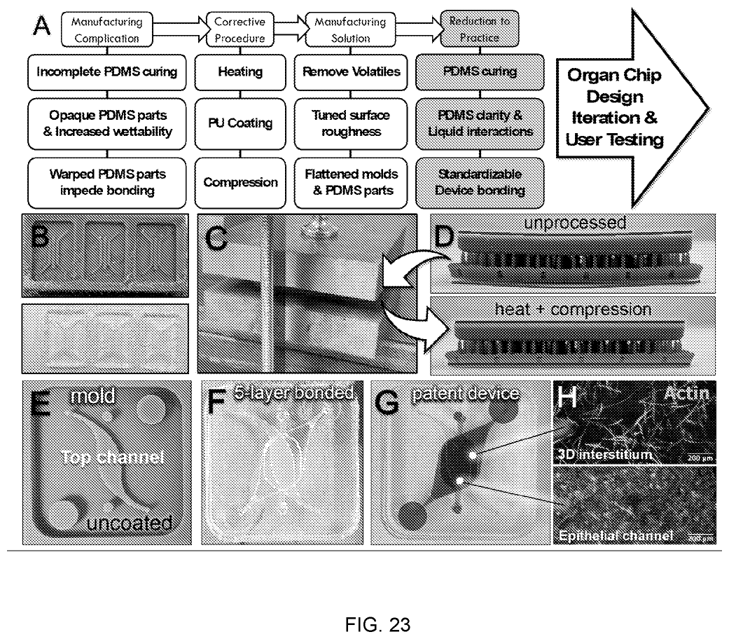

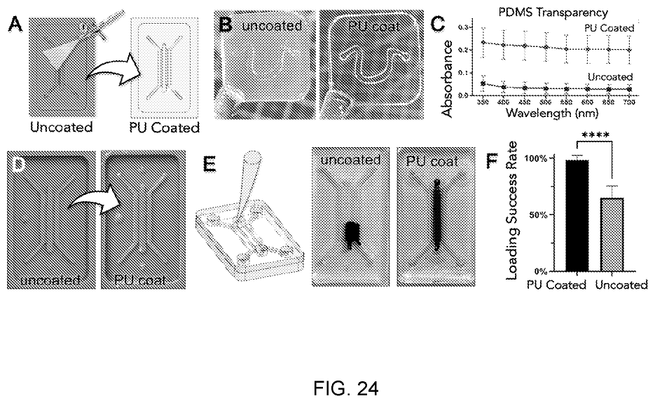

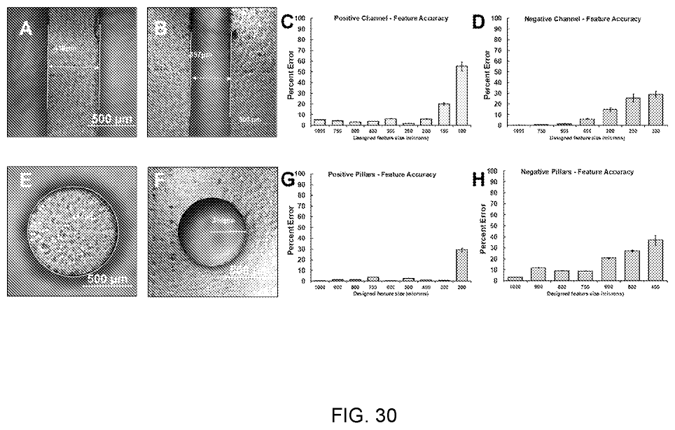

We used a Formlabs F3 SLA printer to leverage rapid prototyping capabilities in the organ chip design and fabrication process. We first tested the resolution of the printer by measuring the accuracy of commonly used mold features such as rectangular channels and cylindrical posts (FIG. 30). We aimed to engineer device-constrained milliscale tissues, and the printer was found to be accurate with positive feature sizes greater than 200 microns, thereby allowing accurate iteration of design features on the intended scale. The resins used for these studies introduced multiple manufacturing challenges (FIG. 23A). Molds stored at ambient temperature prior to PDMS soft lithography retained volatiles that impeded PDMS curing (FIG. 23B). The aspect ratio of organ chip molds exacerbated warping effects and lead to significant mold curvature (FIG. 23D). We baked the molds to first remove volatiles, then we used jeweler blocks heated to 160° C. and a clamping apparatus to flatten molds (FIG. 23C). This manufacturing workaround enabled reduction to practice of complete PDMS curing and standardizable multilayer device bonding using SLA-printed molds (FIGS. 23E and 23F). The PDMS layers produced using these molds are slightly opaque (FIG. 23F). Opacity of molded PDMS is a function of the mold surface roughness, which can be engineered by surface coating (FIG. 24). We engineered a 5-layer organ chip (3 PDMS layers, 2 membranes) for modeling the interface of an epithelium and the underlying interstitium using as-printed rough surface molds (FIG. 23G). This design ensures that the light path of an inverted microscope does not cross molded PDMS before reaching the tissue layers and enables long-working distance imaging of layered tissue structures spanning more than 1 mm in the z-direction (FIG. 23H).

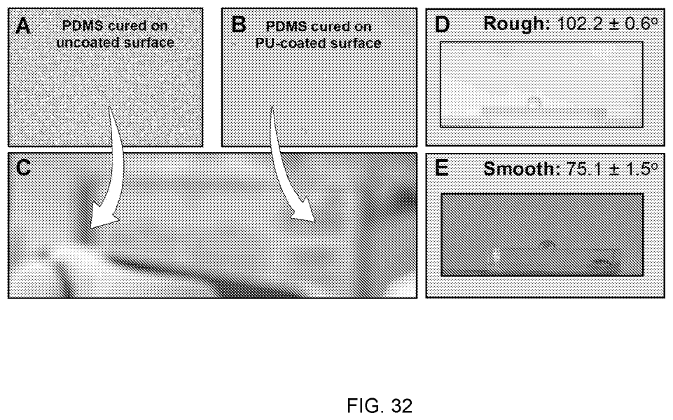

Many organ chip applications require the surface smoothness produced by silicon master molds for reasons of optical clarity and predictable liquid interactions. We developed an adaptable and low-cost method of polyurethane (PU) clear coating that produces molds with a smooth surface finish (FIGS. 24A and 23D). This manufacturing workaround produces molded PDMS parts with near optical clarity as measured on a spectrophotometer (FIGS. 24B and 24C). Membrane-free organ chips (MFOC) function based on control of surface tension in the device.¹⁰ Designed geometry is a primary determinant of surface tension effects, but the PDMS surface influences the process of liquid patterning through surface interactions. We hypothesized that PU-coated mold surfaces would produce more effective liquid patterning in MFOC because rough surfaces are more hydrophobic and will tend to repel injected liquids, thereby counteracting the cohesion needed for stable pinning along the guide structure. We confirmed increased surface roughness and hydrophobicity of PDMS surfaces from uncoated molds using DIC imaging and contact angle measurements (FIG. 32). We used a MFOC design geometry optimized in parallel studies to test injection loading success rates of devices produced with uncoated or PU-coated molds (FIGS. 24D and 24E). Injection of rough-surfaced devices from uncoated molds resulted in more frequent injection loading failures defined by breaching of liquid over internal guide structures (FIG. 24F). PU coating of molds yields MFOC with 98.3+/−4.1% loading success rates compared to 65.0+/−10.5% for rough surfaced MFOC (FIG. 24G).

### Membrane-Free Organ Chip Reduction to Practice

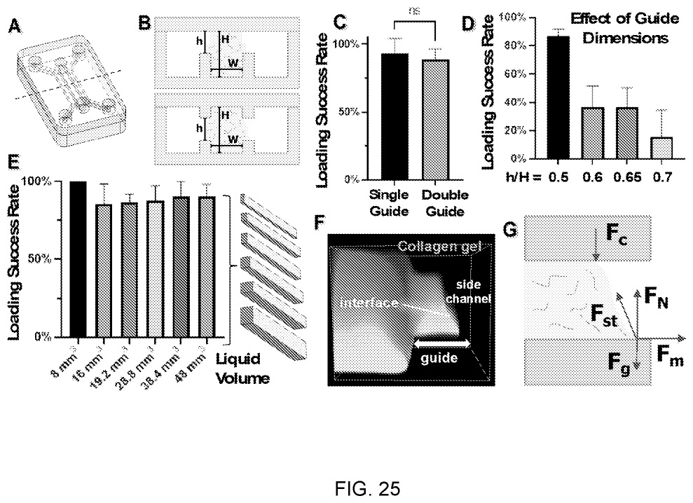

We aimed to engineer MFOC that enable successful injection by a wide range of non-expert users by systematically varying geometric design features and performing user testing (FIG. 25). Microscale MFOC using continuous horizontal guide structures were designed with the guide structure height at 25% the height of the bulk tissue chamber (H), creating an open tissue interface height (h) that is 75% of the bulk tissue height (h/H=0.75)¹¹. We found that h/H=0.75 is not tractable on the millimeter scale in pilot studies based on complete failure to pin liquids in the bulk tissue chamber. We used h/H=0.5 as a starting point for milliscale design iteration and compared single guide and double guide designs (FIGS. 25A and 25B). Both designs enabled near 100% loading efficiency by non-expert users (FIG. 25C). The double guide configuration has the advantage of a centrally positioned interface with all z-direction layers of the bulk tissue situated within 0.25 mm of the open interface.

We tested increased h/H ratios and found that loading efficiency dropped below 40% with h/H=0.6 or 0.65 and dropped below 20% with h/H=0.7 (FIG. 25D). We used h/H=0.5 as a required milliscale MFOC design specification and proceeded to test the effect of increasing the tissue chamber volume, which would be expected to increase the effect of inertial forces derived from the mass of liquid being injected (Fₘ in FIG. 25G). We found that increasing the tissue volume up to 6-fold did not significantly reduce the loading efficiency (FIG. 25E). We did not test larger tissue volumes, but this finding suggests that h/H=0.5 will enable engineering of patterned and fluidically accessible bulk tissues that are sufficiently large to perform a full range of biochemical analyses, such as enzyme activity assays 15. We confirmed that hydrogel precursor solutions load with equal or greater efficiency than water and used FITC-dextran loaded collagen hydrogel to visualize the tissue geometry at the interface situated on top of the MFOC guide structures. Tissues form a foot along the bottom guide that consistently creates a downward sloping geometry at the tissue boundary (FIG. 25F). Failure during loading occurs when the liquid foot breaches over the guide structure during injection (FIG. 25E). We propose that a force balance must occur for stable liquid patterning in which the inertial force generated by the momentum of the injected liquid tending to drive liquid over the guide is balanced by surface tension of the liquid face that depends on designed geometry and cohesion between the liquid and PDMS surface, which is a function of hydrophilicity (Fₘ, Fₛₜ, F_{c} in FIG. 25G).

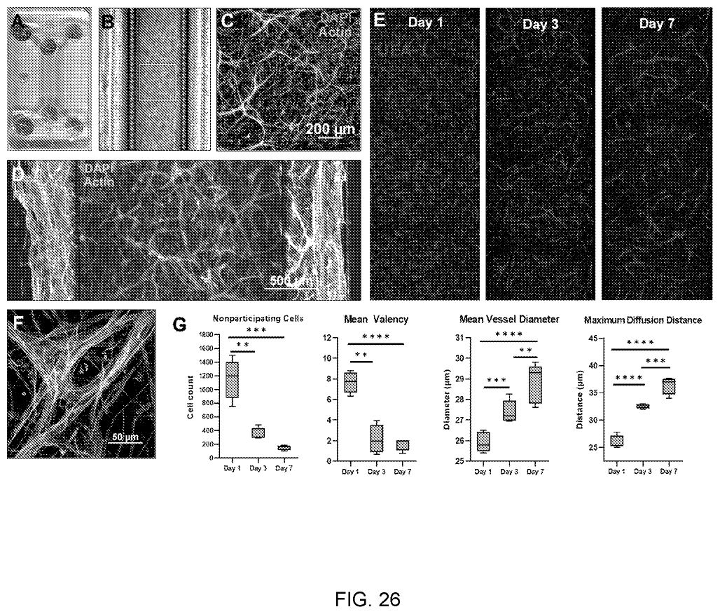

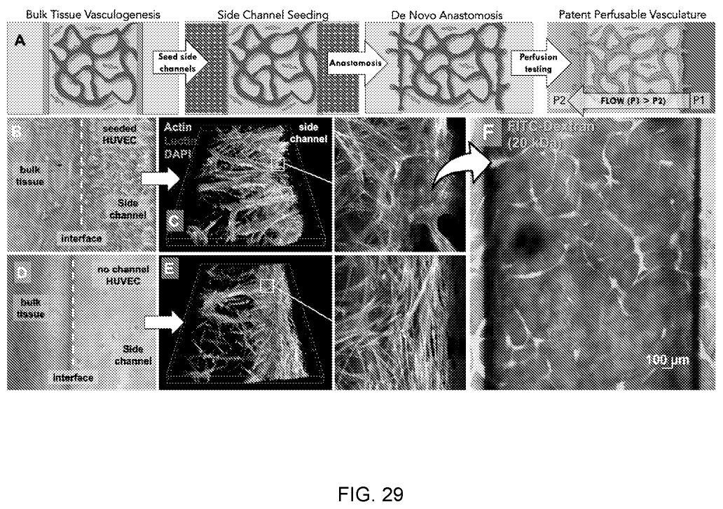

Engineering vascularized tissues is a common endeavor from macroscale tissues intended for implantation to microphysiological systems. We developed milliscale MFOC for applications built around tissue vascularization. The cell-to-liquid volume ratios and reproducible geometry granted by organ chip devices creates an ideal platform for modeling and optimizing tissue-specific bulk vasculogenesis in a physiological environment¹⁶. We engineered parameters of tissue vasculogenesis including densities and ratios of endothelial cells and fibroblasts, and composition of the collagen-fibrin hydrogel blend in bulk interstitial tissues formed in optimized milliscale MFOC (FIGS. 26A and 26B). 3D vascular networks are seen assembling by 3 days in culture (FIG. 26C). Interconnected networks spanning the entire tissue bulk assemble by 7 days in culture with the formation of perivascular niches with closely associated and enrobing fibroblasts (FIGS. 26D and 26F). We used morphometric analysis to quantify metrics such as numbers of non-participating endothelial cells, branching character, vessel size, and spacing between vessels to track network assembly and maturation over 7 days in culture (FIGS. 26E and 26G). The number of non-participating endothelial cells sharply decreases by 3 days and further by 7 days to a reproducibly low fraction (FIG. 26G). Decreasing valency, increasing vessel diameter, and decreased diffusion distance were morphometric features of vascular network maturation into a capillary-like architecture. These studies optimized bulk tissue vascularization in preparation for engineering the process of vascular bed anastomosis for perfusion as shown in FIG. 29.

Patterning Adjacent Bulk Tissues with Tunable Interfacial Properties in MFOC

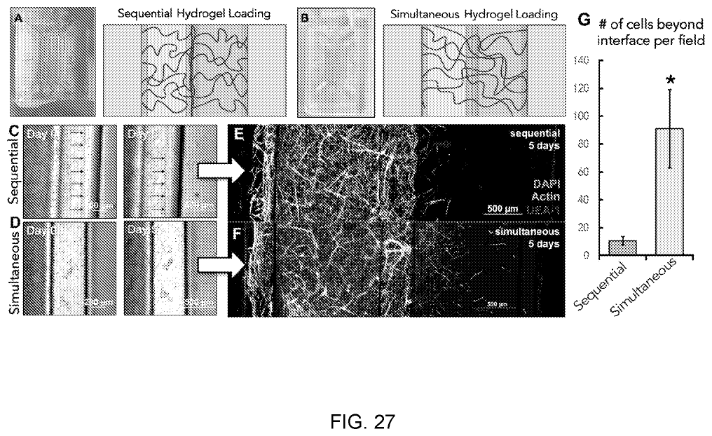

We designed MFOC with two tissue layers separated by a central guide structure to pattern contiguous sequentially-loaded layers of bulk tissue with definable compositions (FIG. 27A). A second design was fabricated with the inlets positioned to allow use of a multichannel pipette for simultaneous loading (FIG. 27B). We hypothesized that allowing the collagen and fibrin hydrogel scaffold of the first tissue lane to completely polymerize will result in a layer of increased ECM density due to dehydration at the air interface, thereby impeding cell migration (FIGS. 27A and 27B, schematics). By contrast, we hypothesized that simultaneous loading of the adjacent hydrogel constructs would result in the formation of a continuous bulk tissue without any interfacial heterogeneities, thereby enabling free cell transit between the tissue layers. In the case of sequential loading, the formation of a dense interfacial layer was visible by DIC imaging immediately after device seeding (Day 0) and persisted to the end-point at 5 days without any visual change in the density, thickness, or continuity of the layer (FIG. 27C, arrows). By contrast, no interfacial layer was visible when the tissue layers were loaded simultaneously (FIG. 27D). We performed UEA-1 lectin and phalloidin whole mount staining after 5 days of culture to visualize transit of fibroblasts and endothelial cells into the empty tissue layer. Profuse migration of fibroblasts and the invasion of endothelial cells and tubular structures was observed in devices with simultaneous loading (FIG. 27F). Conversely, only fibroblasts in smaller numbers were seen in the empty tissue layers of sequentially loaded devices (FIG. 27E). Dense layers of fibroblasts formed with parallel alignment to the interfacial layer (FIG. 27E, arrows). We measured a significant 8-fold increase in the number of transmigrated cells in devices with simultaneous loading (FIG. 27G).

### Fabricating Multi-Tissue Organ Chips for Modeling Premetastatic Niche Cultivation

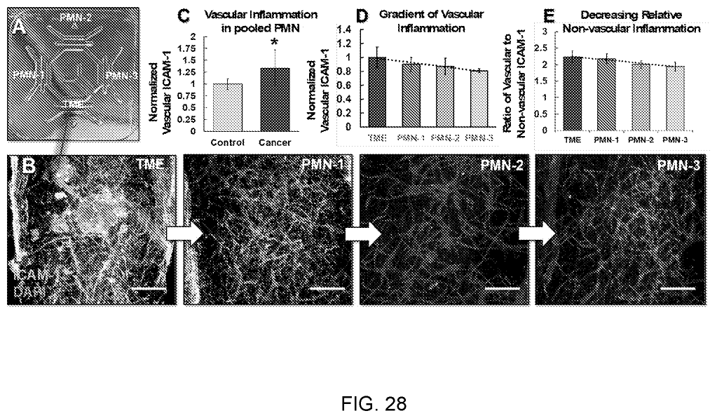

Organ chip devices are easily configured to model interactions between distant tissue and organ niches¹⁶⁻¹⁹ We used single MFOC as the basis for engineering a 4-tissue device to model premetastatic niche (PMN) cultivation elicited by soluble TME-derived factors in 3 PMN situated at increasing distance from the primary TME (FIG. 28A). Vascular inflammation is key component of PMN cultivation that enables transport of serum proteins and trafficking of immune cells to the nascent PMN.^{21,21} We used ICAM-1 as a readout of vascular inflammation.²² Intense ICAM-1 staining was observed throughout the vasculature, tumor cells, and fibroblasts in the primary TME, where the concentration of tumor-derived factors is highest (FIG. 28B, TME). Appreciable ICAM-1 staining was seen in the vasculature and interstitial fibroblasts within the nearest tissue niche (FIG. 28B, PMN-1). ICAM-1 signal intensity in micrographs decreased qualitatively with increasing diffusion distance from the TME (FIG. 28B, PMN-2, PMN-3).

We analyzed the ICAM-1 signal intensity using an algorithm that segregates lectin-stained pixels (uniform staining of endothelial cells), thereby enabling discrimination of ICAM-1 staining localized to endothelial and non-endothelial pixels. Control devices replaced the TME with a fourth PMN to remove tumor-derived factors from the system and create a physiological baseline. We first pooled the 3 PMN for analysis and measured a significant increase in total vascular inflammation with PMNs in devices containing a TME, demonstrating the ability to capture cancer-associated distant vascular inflammation in an engineered tissue model (FIG. 28C). Gradient effects from PMN-1 to PMN-3 were observable and quantifiable. Vascular inflammation decreased following a linear trend, as would be expected in a system with transport governed largely by static diffusion (FIG. 28D). We measured a similar decay in the relative non-vascular inflammation, which also followed a linear trend, indicating that propagation of inflammation in the tissue may scale with the degree of vascular inflammation (FIG. 28E).

Engineering Milliscale Tissues with Perfusable Vasculature

The ability to engineer perfusable internal vasculature enables modeling of dynamic processes associated with vascular physiology, drug delivery, and cell transit in a setting the more accurately mimics the biochemical and physical environment present in vivo. Anastomosis with the internal vasculature and simultaneous endothelialization of all open surfaces of the bulk tissue boundaries facing both side channels are required to achieve a closed system fluidically accessible only via the internal vasculature. We found that seeding the side channels with a high density of endothelial cells after 2 days of bulk tissue vasculogenesis achieves vascular anastomosis which is then confirmed by perfusion testing (FIG. 29A). Endothelial side channel seeding at sufficiently high density (at least 10×10⁶ cells/ml, see Materials and Methods) results in immediate and complete coverage of the tissue interfaces (FIG. 29B). Vascular anastomosis occurs de novo under these conditions, resulting in the formation of an endothelialized tissue boundary that forms several points of anastomosis with the internal vasculature. These anastomoses appear as open pipe faces on a manifold along the length of the tissue boundary (FIG. 29C). Internal vessels are rendered inaccessible in devices without the side channels seeded, due to the formation of dense cell layers at the bulk tissue boundary (FIGS. 29D and 29E). The internal vasculature of bulk tissues anastomosed under these culture conditions was perfusable, and patent, as demonstrated by uniform perfusion of FITC-conjugated dextran without leakage into interstitial spaces (FIG. 29F).

### DISCUSSION

We developed procedures for using low-cost benchtop SLA resin printers to manufacture millifluidic organ chip devices that are widely adaptable for tissue engineering and regenerative medicine research applications. These workflows can be implemented in labs without prior microfabrication experience or access to cleanroom facilities. All molds for organ chip designs shown in this paper can be downloaded and used in the designed form or configured according to user specifications. We established design specifications of geometric features for achieving successful loading of milliscale membrane-free organ chips (MFOC) and determined that bulk tissues can be scaled up to a volume of at least 48 mm³ without any change in loading efficiency (FIGS. 25D and 25E). Resolution limitations are dependent on the model of printer and resins being used and will continue to improve at a rapid pace.² Our post-processing procedures that address resin leachates, mold warping, and surface roughness are applicable to molds with features of any size scale fabricated using other SLA printer models or other types of 3D printers such as extrusion-based printers. PDMS replica molding allows users to overcome the decreased print resolution of negative mold features (FIG. 24).

Multilayer, membrane-partitioned organ chips are ideal platforms for patterning the layered tissue architecture present in most organs.²³ We engineered a universal organ interstitium chip (OIC) using the multilayer configuration that orients a 3D tissue chamber which can filled with user-defined combinations of hydrogels and cell types beneath a membrane on which any cell type may be grown (FIG. 23G). We used the OIC to create a model of stromal reactions in lung cancer in which the 3D interstitial layer contains normal primary human lung fibroblasts in collagen type I hydrogel and the upper channel layer is seeded with lung adenocarcinoma cells (FIG. 23H). Maintaining stable anchorage of the 3D collagen gel housed in OIC requires surface modifications such as coatings that utilize heterobifunctional crosslinking chemistry.^{15,24} Surface engineering approaches can be used to direct patterns of tissue contraction but maintaining bulk tissues in organ chips requires stable anchorage that is challenged by the contractile forces of fibroblasts and other stromal cell types, particularly in the case of collagen type I hydrogel-based constructs.^{15,25} The minimal fabrication requirements for effectively manufacturing our organ interstitium chip or similar models are complete PDMS curing for feature replication and flatness of PDMS layers to enable reproducible bonding of device layers and place cell and tissue layers in horizontal imaging planes. Our post-processing workflow for 3D printed organ chip molds helps to overcome the common problems of distortion when rapid prototyping parts with sub millimeter feature resolutions and incomplete PDMS curing when using 3D printed molds, both major impediments of effective device manufacturing.²⁶

The clarity of microfluidic devices is derived from the smooth surfaces of silicon master molds generated by photolithography in clean room settings^{27,28} We successfully manufactured devices with near perfect optical clarity using rough surfaced molds refinished with a polyurethane clear coat (FIG. 24). The use of widely available automotive refinishing products ensures that this method is adaptable in any setting where ventilation is controlled. Our study demonstrates that surface finish also affects liquid interactions in PDMS organ chips, which is of importance for designs that leverage surface tension for membrane-free liquid patterning such as the MFOC presented in this paper (FIG. 24F). The increased loading failure rates of MFOC molded from uncoated molds with rough surfaces can be explained by theory demonstrating that the higher surface energy of rough surfaces increases hydrophobicity, which was confirmed by our contact angle measurements, and would be expected to increase liquid repellence and promote cavitation that could lead to liquid spilling over the guide (32).^{29,30} for MFOC loading, strong cohesion of the liquid with the PDMS guide structure facilitated by a smooth, more hydrophilic surface is desired to promote pinning of liquid along the guide. PU coating of MFOC molds is a necessary step for reduction to practice on the level of user injection loading tests. (FIG. 24F). Our MFOC reduction to practice study ensures that use of the designs reported in this paper will enable reproduction of similar models with user-defined cell compositions in any lab with prior experience performing multicellular 3D hydrogel cultures.

Organ chip models of angiogenesis provide patterned systems that enable precise definition of tissue geometry and quantification of directional cell migration processes.³¹ Our 2-lane MFOC device enables modeling of angiogenic processes and bulk cell migration in the setting of definable and continuous tissue interface without the presence of vertically spanning PDMS structures that render the tissue interface discontinuous and introduce regions of mechanical heterogeneity. The primary tissue engineering capability of 2-lane MFOC is the ability to tune properties of the interfacial layer by varying the sequence of injection loading and hydrogel polymerization (FIG. 27). The sequential loading methods will allow users to engineer interfaces with defined heterogeneity (FIGS. 27A, 27C, and 27E). The density and thickness of the interfacial layer observed in our experiments is likely tunable by changing the duration of gel polymerization, and therefore surface layer dehydration, prior to introducing liquid medium. Our results demonstrate the ease of modeling the impact of interfacial ECM density on cell migration processes (FIGS. 27E and 27G). The finding that vascular invasion across tissue interfaces is impeded by interfacial heterogeneity has many implications for tissue engineering approaches that involve docking of sequentially polymerized tissue volumes. The 2-lane device is modular by design to enable the creation user-defined and tissue-specific assays of cell transit and tissue structure invasion across engineered tissue interfaces.

MPS technology has the potential to accelerate and improve in vitro cancer research through the creation of models that capture some complexities of cancer pathophysiology via the fluidic interconnection of multiple tissue compartments. Inflammatory activation originating in the primary tumor microenvironment that propagates locally, regionally, and at distant organ locations is a key aspect of cancer pathophysiology.³² Inflammatory activation is mediated through the transport of proinflammatory molecules through the vasculature where they elicit upregulation of endothelial adhesion molecules including ICAM-1.³³ We previously established a microphysiological model of tumor cytokine-driven muscle tissue injury using a device that fluidically interconnects an engineered tumor microenvironment with patterned muscle tissue constructs.¹⁵ Here we expanded those studies by engineering a 4-compartment model that captures a distance-dependent degree of vascular inflammation in the presence of a primary tumor microenvironment (FIG. 28). Tissue engineered models capable of capturing gradient effects have many potential applications in regenerative medicine research. Our study demonstrates gradient effects of tumor-derived factors on vascular inflammation, but the same device platform could be used to study proangiogenic or cell-fate modulating effects in a model configured to study induction of regenerative processes.

Microfluidic models with perfusable vasculature have been reported in numerous formats for more than a decade.³⁴ We built off these designs, scaled them up to millimeter dimensions and established standardized and widely adaptable procedures from device manufacturing to formation and testing of perfusable internal vasculature in milliscale bulk tissues (FIG. 29). Previous protocols reported the use of complex sequences of switching VEGF gradients and interstitial flow gradients to achieve vascular anastomosis and patent perfusion.¹² Our method only requires a two-step device seeding protocol and static culture using standard culture medium formulations. This capability has great potential for the creation of models to study transport processes in tissues associated with drug delivery, circulating cell homing and transmigration, and changes in vascular permeability. It is important to note that while we achieved formation of patent vascular networks in a static culture system, there are natural fluctuations of pressure that drive flow in the anastomosed vessels, and mechanical conditioning of engineered microvessels via flow induced shear stress has been shown increase the maturation of endothelial barriers.^{35,36} Our MFOC platform can be easily adapted to continuous or recirculating flow systems, although without the ability to precisely control flows and shear stresses throughout the bulk vasculature due to the stochasticity of the network architecture. Future work will focus on developing a multiorgan MFOC model in which the separate compartments are connected via a continuous vasculature that spans the bulk tissue of each compartment.

### CONCLUSION

Microphysiological models such as organ chips have demonstrated the ability to capture complex physiological and pathophysiological processes, but their adoption in non-expert laboratories has been limited by the technical barriers of microfabrication and culture in microfluidic device formats. As demonstrated herein, it is possible to design and manufacture organ chip devices capable of modeling complex millimeter scale tissue processes in any benchtop laboratory setting. The creation of these models in laboratories accustomed to performing organoid cultures in common 3D matrices such as plate bound Matrigel or collagen gel will grant the capability to precisely control tissue volume, vascularization, and transport processes. Similarly, the designs reduced to practice through the studies presented in this paper create a low barrier for entry into modeling pathophysiological processes that require interaction of multiple tissue niches. These models can create a new filter for hypothesis testing and target validation in the preclinical pipeline that sits between high-throughput plate based systems and animal models commonly used non-engineering preclinical laboratories. Limitations of the current manufacturing approach are primarily related to attainable printer resolution. This is an important economic consideration since higher resolution printers are more expensive. Printer cost will continually decrease with concomitant improvements in printable feature resolution. We have demonstrated that current hobbyist level SLA printers can be used to manufacture milliscale organ chip systems with product-level performance. These benchtop manufacturing capabilities will facilitate the development of more complex in vitro culture systems to accelerate and increase the relevance of studies aimed at understanding the mechanisms of human physiology and develop improved therapies, such as anti-metastasis therapies for patients with advanced malignancy.

### Methods

### Experimental Design

The first phase of our study aimed to establish reproducible workflows for SLA-based manufacturing of organ chips that grants the core capabilities of silicon master molds fabricated by photolithography in clean room settings. We then used the established manufacturing workflow to perform rapid prototyping and iterate the design of milliscale membrane-free organ chip (MFOC) designs with the goal of optimizing loading efficiency of the devices by a range of users. Optimized MFOC designs were used to optimize bulk tissue vasculogenesis in milliscale MFOC, demonstrate the capacity to model distant tissue interactions via interconnection of multiple MFOC, and establish reproducible and adaptable procedures for producing perfusable and patent internal vasculature within bulk tissues in milliscale MFOC. All organ chip design specifications and STL files for the molds are available in the online

### Supplementary Information.

### SLA Printing

Device molds were drafted as 3D drawings using SolidWorks (Dassault Systèmes) or Fusion 360 (Autodesk). Molds were created in a top-down view and printed using a Form 3B SLA Printer (Formlabs). Final designs were exported at “.SLT” files to Formlab's PreForm software.

Formlab's PreForm software was used to prepare 3D drawing files for printing. Each part file was oriented so that the mold base was parallel with the printer's build platform. Print supports were autogenerated within PreForm with a 0.65 mm touchpoint size and a 1.30 support density. All molds were printed using Formlab's proprietary ‘Grey’ or ‘Clear’ SLA resins.

### Mold-Post-Processing

Completed prints were washed in isopropyl alcohol (IPA) according to Formlabs protocols for the resins used, and then washed in IPA for 20 additional minutes in the FormWash (Formlabs). Post wash, molds were dried until IPA was completely evaporated. Molds were then dried and cured under UV light (FormCure, Formlabs) at 60° C. Clear resin parts were cured for 15 minutes. Grey resin parts were cured for 30 minutes. Warping caused by distortion of the printing process and the curing process was corrected by first baking Clear resin molds at 130° C. for 2 hours and baking Grey resin molds for 3 hours. For the last 30 minutes of bake time, 2 stainless steel jeweler's blocks were added to the oven to heat. Molds were removed and placed between two jewelers blocks with or without clamping. Flatness of the parts was assessed visually relative to a straight edge. We used a commercial painting airbrush (Model 105 Patriot Fine Gravity Airbrush, Badger Airbrush Co.) to coat molds with lacquer thinner (Klean-Strip). Parts were dried for 15 minutes while preparing automotive clear coat (Finish 1 FC720/FH612, Sherman Williams) mixed at a 1:4 ratio of hardener to clear coat. The mixture was airbrushed from approximately 8 inches away using a continuous back and forth motion and then a continuous up and down motion until a thin layer of clear coat was visible. 4 layers were applied with the mold rotated 90° between applications. Coated molds were dried for 6 hours before silanization according to standard protocols for PDMS soft lithography³⁷.

### PDMS Soft Lithography

PDMS (Sylgard 184, Ellsworth Chemical) was mixed at a 1:10 ratio of PDMS curing agent to PDMS elastomer by weight and degassed. PDMS soft lithography was carried out according to standard protocols used for silicon master molds.³⁸ To produce PDMS molds with two flat surfaces, we applied a cleaned 2×3 inch glass slide to the top of the mold to sandwich the uncured PDMS, carefully avoiding bubble formation. A jeweler's block was placed on top of the slide to ensure a tight seal and filled molds were baked at 60-° C. for 4 hours.

### Replica Molding

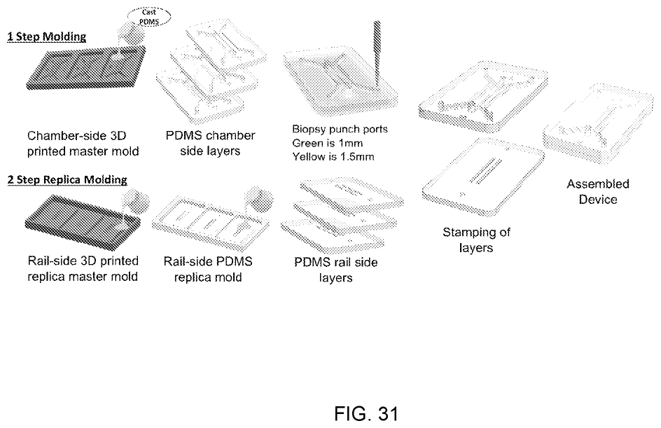

Double guide MFOC designs used for these studies required a second mold generated using PDMS replica molding, due to negative feature resolution limitations of our SLA printer. Instead of the 3D printed mold being the complement of the final PDMS part, the mold was a replica of the desired PDMS part (FIG. 31). The PDMS replica mold of the eventual MFOC device layer was created using the PDMS soft lithography methods described above. The PDMS replica mold part was silanized, plasma treated and used for molding device layers.

### Device Assembly

Cured PDMS device layers were removed from molds and detached from the glass slides using a razor blade. Clear packing tape was used to remove any dust from the PDMS parts. Through features, such as inlets, were removed using biopsy punches and an X-acto knife as needed. Device bonding was achieved by PDMS stamping as reported previously³⁹. Briefly, 3 g of 1:10 PDMS was spin coated to create a thin stamping layer. Device layers were then stamped onto this PDMS layer and then stacked together. Reproducibility of layer alignment was ensured by the inclusion of Lego-like press fitting features. Assembled devices were cured at room temperature for at least 24 hours.

### Injection Testing

To test the usability of each design iteration, the devices underwent a series of injection testing. In each, at least 7 users with varying levels of familiarity with injecting microfluidic devices were recruited. For each design, 3 devices were fabricated. We used 200 ml pipettes with standard yellow tips to inject water colored with food dyes. Following injection, the water was removed from the device, and the device was completely dried using compressed air. If injected water reached the outlet without breaching into side channels, the trial was successful. The number of successful trails and number of unsuccessful trails were recorded. Each user completed 10 tests per design. 100 μg/mL 40 kDa FITC-dextran (Sigma-Aldrich) at was mixed into a collagen hydrogel prepared as described above and injected into the central channel. We acquired 3D LSCM stacks to visualize tissue interfaces on the guide structures.

### Cell Culture

Human umbilical vein endothelial cells (HUVEC) (ATCC) were maintained in Vascular Cell Basal Medium supplemented with Endothelial Cell Growth Kit-VEGF (ATCC). Human lung fibroblasts (HLF) were maintained in Fibroblast Basal Medium supplemented with fibroblast Growth Kit-Low Serum (ATCC). A549 lung adenocarcinoma cells were cultured in F12 medium with 10% FBS.

All culture media contained 1% antibiotic-antimycotic (Corning). Cells were maintained in a humidified tissue culture incubator maintained at 37° C. and 5% C02 and used at passages 3-7.

### Organ Chip Seeding

Organ chips were exposed to UV light in a cell culture hood for 1 hour. The surfaces of chambers of multilayer devices and lanes of MFOC that house bulk 3D tissues were functionalized for ECM hydrogel anchorage using the polydopamine (PDA) coating method as previously described²⁴. Briefly, dopamine solution was prepared by mixing 10 mg of dopamine hydrochloride (Sigma-Aldrich)—with 10 mM Tris-hydrochloride (Sigma-Aldrich). Dopamine solution was sterile filtered and injected in the tissue chambers and lanes of devices. Devices were incubated for 2 hours at room temperature in the dark, aspirated and used immediately. Multilayer membrane-bound organ chips were loaded with collagen type I hydrogel (2.5 mg/ml) containing primary normal human lung fibroblasts (5×10⁵ cells/ml) in the 3D tissue chamber and the upper tissue layer channel was seeded with A549 lung adenocarcinoma cells (1×10⁶ cells/ml). MFOC were seeded with HLF and HUVEC (2×10⁶ cells/mL each) admixed in 2.25 mg/mL collagen I (Corning), 5 mg/mL fibrinogen (Sigma-Aldrich), and 1 U/mL thrombin (Sigma-Aldrich). This cell inoculated hydrogel precursor was injected into central tissue lane of MFOC devices and incubated for 30 minutes prior to filling the side channels and media reservoirs with endothelial cell growth medium (VEGF containing kit) with 25 μg/mL aprotinin (Sigma-Aldrich) for culture. 2-lane MFOC were seeded in the manner. Sequential injection was performed by loading the first tissue layer and allowing 30 minutes of polymerization prior to injection of the adjacent tissue layer. Simultaneous injection was performed using a multichannel pipette. In both cases, one layer of tissue was comprised of the same hydrogel devoid of cells.

### Whole Mount Staining of Organ Chips

Tissue layers and bulk tissues in organ chips were stained using adaptations of previously reported protocols^{15,23,40} Briefly, tissues were fixed by loading 4% paraformaldehyde in the liquid channels and incubating for 1 hour at room temperature then overnight at 4° C. Devices were washed with PBS and stored at 4° C. prior to staining. Tissues were stained using 4 mL/mL 4′,6-diamidino-2-phenylindole (DAPI) to label nuclei, 4 mL/mL phalloidins to label actin in all cells, and 20 mL/mL Ulex Europeas agglutinin I (UEA-1) to specifically label endothelial cells⁴¹. The staining cocktail was prepared in 1×PBS with 0.2% Triton-X and 1% BSA. Devices were loaded with the staining cocktail, rocked gently for 1 hour at room temperature, refrigerated overnight, and rocked for an additional hour at room temperature before washing. Stained devices were loaded with PBS and stored at 4° C. protected from light until imaging.

### Image Analysis

Stained tissues were fixed in position on a slide and imaged on an inverted Nikon C2 laser scanning confocal microscope (LSCM) equipped with a Nikon DS-FI3 camera. Max intensity projection Z-stacks were exported as TIFFs with no LUTs attached. All Image analysis was completed in MATLAB (R2021b). Vascular network images were then smoothed using an edge preserving filter with a gaussian kernel, and a threshold was applied to remove remaining low-intensity noise. All Image analysis was completed in MATLAB (R2021b). We used a pretrained deep neural network to denoise each image and adaptive histogram equalization was used to standardize contrast across the image set⁴². We used a circular Hugh transform to identify rounded cells in each image and counted rounded cells to assess network completeness (Atherton & Kerbyson, 1999). Rounded cells were removed from images prior to segmentation and morphometric analysis. We smoothed images using an edge preserving filter with a Gaussian kernel and applied a threshold to remove remaining low-intensity noise (Tomasi & Manduchi, 1998). We then segmented pre-processed images and quantified morphometric parameters using an open-source automated segmentation tool 43. All devices stained with ICAM-1 were imaged at a fixed laser intensity, and mean pixel intensity was computed for each image. ICAM-1 localization to endothelial cells was discriminated by colocalization with UEA-1 lectin. Mean fluorescence intensity was calculated for the total image, UEA-1 co-localized pixels (vascular inflammation), and pixels with ICAM-1 signal only (non-vascular inflammation). For cell enumeration, Hoechst-labeled nuclei were segmented and counted as particles in FIJI.

### Establishing Anastomosed and Perfusable Bulk Vasculature

After two days of bulk tissue vasculogenesis initiated as described above, HUVEC (10×10⁶ cells/mL) were injected into the MFOC side channels. Devices were maintained in static culture for another 7 days with media changes every 24 hours to allow for lumen formation and vessel maturation. We define static culture as no pumping or rocking but slight differences in liquid head height of medium reservoirs lead to trickling flows in the anastomosing vasculature. One channel was loaded with 100 μg/mL of 20 kDa FITC-dextran (Sigma-Aldrich) in PBS with the reservoirs filled to an elevated pressure head and allowed to perfuse through vasculature until it reached equilibrium with the opposite channel. Videos were acquired as time series of the DIC and FITC channels using the Nikon C2 LSCM. 3D stacks were acquired to visualize patency of the vasculature throughout the 3D bulk tissue.

### Statistical Analysis

All vasculogenesis experiments were repeated n=3 times. A large, composite 1×6 stitched image was acquired from each sample to ensure convergence of morphological variables. Images were preprocessed and segmented per above image analysis methodology. All statistical analyses were completed in GraphPad Prism V 9.2. Comparison of MFOC injection loading success rates, comparison of morphometric parameters between days of vascular network formation, ICAM-1 fluorescence intensity in PMN model, and comparison of transmigrated cell numbers in the 2-lane model were made using unpaired t-tests with a 95% confidence limit for statistical significance, i.e. P<0.05. Sample sizes and the number of independent experiments performed are indicated in the respective figure captions.

### Example 3. Engineered Tumor Tissues Induce Local and Distant Vascular Inflammation in a Multicompartment Microphysiological Model of Premetastatic Niche Cultivation

Introduction: Advanced malignancy involves systemic processes associated with disease progression and physiological decline. Metastasis involves the process of premetastatic niche (PMN) cultivation by which distant tissue microenvironments are rendered receptive to tumor cell arrival. Cachexia is a multifactorial syndrome characterized by muscle wasting and dysfunction in multiple organs. Cachexia and PMN cultivation are increasingly linked in the oncology literature with a common theme of cytokine-driven inflammation underlying the pathophysiology. Previous work established a microphysiological model of skeletal muscle injury driven by tumor-derived factors including proinflammatory TNF1. Here we present a four compartment microphysiological system (MPS) designed to study tumor-driven vascular pathology locally and in downstream organ compartments. We demonstrate that the presence of lung cancer significantly increases vascular inflammation.

Materials and Methods: polydimethylsiloxane (PDMS) devices were fabricated using SLA printed molds and standard soft lithography. The tissue compartments of each organ module were 3 mm wide, 6.5 mm long, and 3 mm high. The membrane free design utilizes top and bottom guide structures (0.285 mm wide, 0.25 mm high) that separate the central tissue lanes and the flanking fluidic channels. In this study we formed prototype stromal vascular tissues by loading human lung fibroblasts (HLFs) and human umbilical vein endothelial cells (HUVECs) (2×106/ml each) in a collagen type I and fibrin hydrogel blend and allowing 3D vasculogenesis to occur throughout the tissue volumes. Tumor spheroids comprised of A549 lung cancer cells (LCC) were formed in low attachment plates and added to the stromal vascular mixture for seeding in the tumor compartment of our devices (TME, FIG. 1A). On Day 7 of culture, devices were fixed and tissues were whole-mount immunostained for the proinflammatory adhesion molecule ICAM-1 with ulex europeas lectin (Lectin) as a counterstain to label endothelial cells. Custom image analysis algorithms developed in MATLAB were used to quantify total ICAM-1 fluorescence intensity in 3D image stacks. Vascular ICAM-1 expression was quantified by masking the signal colocalized with Lectin.

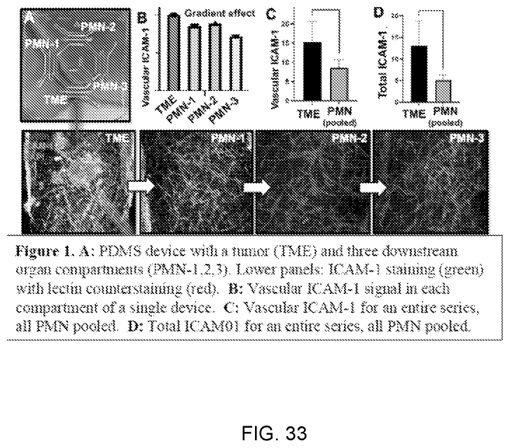

Results and Discussion: Several rounds of design iterations were conducted to produce devices that enable nearly 100% successful injections and maintenance of stably anchored tissues. Our previously optimized tissue vasculogenesis protocol enabled reproducible complete vascularization of each organ compartment. The incorporation of lung cancer tissue in the model induced robust upregulation of ICAM-1 indicative of vascular inflammation. The effect was most potent locally in the chamber containing the cancer tissues (FIG. 33A, TME). Results varied among devices; however, we measured an emerging gradient of inflammation as a function of distance from the source of tumor-derived factors (FIG. 1B). While gradient effects in the sequence of PMN compartments remains statistically inconclusive, pooling the PMN compartments (FIG. 33A, PMN-1,2,3) for comparison with the TME compartment revealed a significant increase in the local (TME) vascular ICAM-1 signal compared to distant organ compartments (PMN pooled) (FIG. 33C). The difference in total ICAM-1 was more pronounced than vascular ICAM-1 due to the contribution of inflamed fibroblasts and tumor cells in the TME compartment (FIG. 33D).

Conclusion: Our study demonstrates the ability of MPS to capture local and distant vascular inflammation induced by tumor-derived factors and establishes an engineering framework for modeling the effect of cancer type and treatment on history on local and distant inflammatory activation in engineered organ compartments.

### Example 4—Engineering a Milliscale Vascularized Model of Patient-Derived Triple Negative Breast Cancer

Introduction: Triple negative breast cancer (TNBC) is a lethal disease that features a prominent health disparity amongst African American (AA) women. Our objective is to develop a fully vascularized microphysiological model of TNBC using patient biopsies from the New Orleans community where AA women experience high rates of incidence and mortality. In addition to modeling disease progression, there is an urgent need to preserve and expand patient biopsy material in defined animal-free systems. To address this need, we manufactured a milliscale membrane free organ chip (MFOC) that enables reproducible loading and rapid formation of perfusable vasculature spanning tissues greater than 1 mm in the shortest dimension. In parallel, we developed methods of TNBC biopsy processing that yield injectable explants for MFOC seeding to create the integrated microphysiological model.

Materials and Methods: MFOC molds were manufactured with a benchtop stereolithography (SLA) resin printer (F3, Formlabs) and application-specific in house workflow. MFOC were fabricated by standard polydimethylsiloxane (PDMS) soft lithography and PDMS stamping for multilayer bonding. A design-test-iterate approach was applied to optimize MFOC geometry and achieve reduction to practice, which we defined as 100% success rate loading devices (no blowout failure) and achieving milliscale tissue vascularization in 5 days. For model development, rapid tissue vascularization was achieved by loading human lung fibroblasts (HLFs) and human umbilical vein endothelial cells (HUVECs) in a collagen and fibrin hydrogel blend and seeding of the gel walls with endothelial cells. Homogenization of patient-derived tumor tissues is accomplished is less than 1 hour by mechanical and enzymatic dissociation with a cocktail of collagenase, dispase, and DNase, followed by filtering through a 250 μm mesh to yield a preparations of tissue fragments. We engineered TNBC tissue density in MFOC by varying the volume ratio of tumor homogenate and collagen-fibrin gel. TNBC tissue viability and proliferation in MFOC was assessed by Live/Dead and Ki67 staining, respectively. When tumors were unavailable, spheroids of patient-derived TNBC cell lines were used to optimize formation of a perfusable vascularized model. Vascular network morphogenesis and anastomosis were visualized by 3D confocal imaging of tissues stained with endothelial-specific lectins. Anastomosis and network patency were further confirmed by FITC-dextran perfusion.

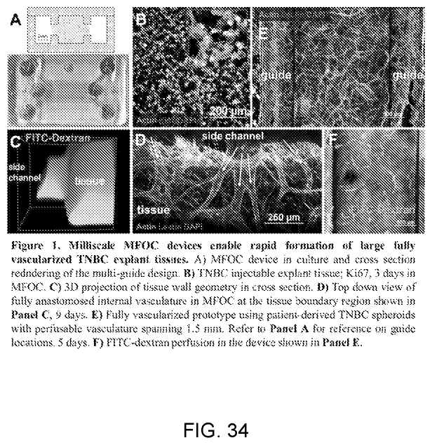

Results and Discussion: A novel milliscale MFOC design with multiple guide structures enabled 100% liquid pinning success rates of volumes up to 1.5×2×12 mm (FIG. 1A). This capability enables rapid production of vascularized tissues with volumes up to 36 μm³; an order of magnitude greater than tissue volumes in microscale membrane-free systems. Platforms that allow for tumor growth in vitro will be required to replace xenograft models. Our current tumor dissociation and processing protocol yields partially-digested injectable homogenates that form dense, viable TNBC tissues with large fractions of Ki67-positive proliferating cells in short-term MFOC culture (FIG. 34B). We characterized the tissue boundary geometry in MFOC (FIG. 34C) and established a method for rapid tissue wall endothelialization and complete vascular network anastomosis (FIGS. 34D, E, F) that does not require chemical or fluid flow gradients which are difficult to implement and standardize.

Conclusions: Our MFOC design enables rapid production of fully vascularized milliscale TNBC explant tissues that will enable preservation and expansion of this precious clinical resource and simultaneous investigation of potential biological underpinnings of the TNBC health disparity in the New Orleans community. Ongoing work is focused on characterizing TNBC phenotypic heterogeneity upon biopsy expansion and passaging in MFOC, and standardizing drug delivery to the TNBC microenvironment via living vasculature in MFOC.

TABLE X
Sequences

| | |
|---|---|
| GAPDH | MAPQMYEFHLPLSPEELLKSGGVNQYVVQEVLSIKHLPPQLRAFQAAFRAQGPLAMLQHFDTIYSI |
| | LHHFRSIDPGLKEDTLQFLIKVVSRHSQELPAILDDTTLSGSDRNAHLNALKMNCYALIRLLESFET |
| | MASQTNLVDLDLGGKGKKARTKAAHGFDWEEERQPILQLLTQLLQLDIRHLWNHSIIEEEFVSLVT |
| | GCCYRLLENPTINHQKNRPTREAITHLLGVALTRYNHMLSATVKIIQMLQHFEHLAPVLVAAVSL |
| | WATDYGMKSIVGEIVREIGQKCPQELSRDPSGTKGFAAFLTELAERVPAILMSSMCILLDHLDGEN |
| | YMMRNAVLAAMAEMVLQVLSGDQLEAAARDTRDQFLDTLQAHGHDVNSFVRSRVLQLFTRIVQ |
| | QKALPLTRFQAVVALAVGRLADKSVLVCKNAIQLLASFLANNPFSCKLSDADLAGPLQKETQKLQ |
| | EMRAQRRTAAASAVLDPEEEWEAMLPELKSTLQQLLQLPQGEEEIPEQIANTETTEDVKGRIYQLL |
| | AKASYKKAIILTREATGHFQESEPFSHIDPEESEETRLLNILGLIFKGPAASTQEKNPRESTGNMVTG |
| | QTVCKNKPNMSDPEESRGNDELVKQEMLVQYLQDAYSFSRKITEAIGIISKMMYENTTTVVQEVIE |
| | FFVMVFQFGVPQALFGVRRMLPLIWSKEPGVREAVLNAYRQLYLNPKGDSARAKAQALIQNLSLL |
| | LVDASVGTIQCLEEILCEFVQKDELKPAVTQLLWERATEKVACCPLERCSSVMLLGMMARGKPEI |
| | VGSNLDTLVSIGLDEKFPQDYRLAQQVCHAIANISDRRKPSLGKRHPPFRLPQEHRLFERLRETVTK |
| | GFVHPDPLWIPFKEVAVTLIYQLAEGPEVICAQILQGCAKQALEKLEEKRTSQEDPKESPAMLPTFL |
| | LMNLLSLAGDVALQQLVHLEQAVSGELCRRRVLREEQEHKTKDPKEKNTSSETTMEEELGLVGA |
| | TADDTEAELIRGICEMELLDGKQTLAAFVPLLLKVCNNPGLYSNPDLSAAASLALGKFCMISATFC |
| | DSQLRLLFTMLEKSPLPIVRSNLMVATGDLAIRFPNLVDPWTPHLYARLRDPAQQVRKTAGLVMT |
| | HLILKDMVKVKGQVSEMAVLLIDPEPQIAALAKNFFNELSHKGNAIYNLLPDIISRLSDPELGVEEE |
| | PFHTIMKQLLSYITKDKQTESLVEKLCQRFRTSRTERQQRDLAYCVSQLPLTERGLRKMLDNFDCF |
| | GDKLSDESIFSAFLSVVGKLRRGAKPEGKAIIDEFEQKLRACHTRGLDGIKELEIGQAGSQRAPSAK |
| | KPSTGSRYQPLASTASDNDFVTPEPRRTTRRHPNTQQRASKKKPKVVFSSDESSEEDLSAEMTEDE |
| | TPKKTTPILRASARRHRS |
| aSMA | MCDEDETTALVCDNGSGLVKAGFAGDDAPRAVFPSIVGRPRHQGVMVGMGQKDSYVGDEAQSK |
| | RGILTLKYPIEHGIITNWDDMEKIWHHTFYNELRVAPEEHPTLLTEAPLNPKANREKMTQIMFETFN |
| | VPAMYVAIQAVLSLYASGRTTGIVLDSGDGVTHNVPIYEGYALPHAIMRLDLAGRDLTDYLMKIL |
| | TERGYSFVTTAEREIVRDIKEKLCYVALDFENEMATAASSSSLEKSYELPDGQVITIGNERFRCPETL |
| | FQPSFIGMESAGIHETTYNSIMKCDIDIRKDLYANNVMSGGTTMYPGIADRMQKEITALAPSTMKI |
| | KIIAPPERKYSVWIGGSILASLSTFQQMWITKQEYDEAGPSIVHRKCF |
| SMAD2 | MSSILPFTPPVVKRLLGWKKSAGGSGGAGGGEQNGQEEKWCEKAVKSLVKKLKKTGRLDELEKA |
| | ITTQNCNTKCVTIPSTCSEIWGLSTPNTIDQWDTTGLYSFSEQTRSLDGRLQVSHRKGLPHVIYCRL |
| | WRWPDLHSHHELKAIENCEYAFNLKKDEVCVNPYHYQRVETPVLPPVLVPRHTEILTELPPLDDY |
| | THSIPENTNFPAGIEPQSNYIPETPPPGYISEDGETSDQQLNQSMDTGSPAELSPTTLSPVNHSLDLQP |
| | VTYSEPAFWCSIAYYELNQRVGETFHASQPSLTVDGFTDPSNSERFCLGLLSNVNRNATVEMTRRH |
| | IGRGVRLYYIGGEVFAECLSDSAIFVQSPNCNQRYGWHPATVCKIPPGCNLKIFNNQEFAALLAQS |
| | VNQGFEAVYQLTRMCTIRMSFVKGWGAEYRRQTVTSTPCWIELHLNGPLQWLDKVLTQMGSPSV |
| | RCSSMS |
| MLCK | MDTKLNMLNE KVDQLLHFQE DVTEKLQSMC RDMGHLERGL HRLEASRAPG PGGADGVPHI |
| | DTQAGWPEVL ELVRAMQQDA AQHGARLEAL FRMVAAVDRA IALVGATFQK SKVADFLMQG |
| | RVPWRRGSPG DSPEENKERV EEEGGKPKHV LSTSGVQSDA REPGEESQKA DVLEGTAERL |
| | PPIRASGLGA DPAQAVVSPG QGDGVPGPAQ AFPGHLPLPT KVEAKAPETP SENLRTGLEL |
| | APAPGRVNVV SPSLEVAPGA GQGASSSRPD PEPLEEGTRL TPGPGPQCPG PPGLPAQARA |
| | THSGGETPPR ISIHIQEMDT PGEMLMTGRG SLGPTLTTEA PAAAQPGKQG PPGTGRCLQA |
| | PGTEPGEQTP EGARELSPLQ ESSSPGGVKA EEEQRAGAEP GTRPSLARSD DNDHEVGALG |
| | LQQGKSPGAG NPEPEQDCAA RAPVRAEAVR RMPPGAEAGS VVLDDSPAPP APFEHRVVSV |
| | KETSISAGYE VCQHEVLGGG RFGQVHRCTE KSTGLPLAAK IIKVKSAKDR EDVKNEINIM |
| | NQLSHVNLIQ LYDAFESKHS CTLVMEYVDG GELFDRITDE KYHLTELDVV LFTRQICEGV |
| | HYLHQHYILH LDLKPENILC VNQTGHQIKI IDFGLARRYK PREKLKVNFG TPEFLAPEVV |
| | NYEFVSFPTD MWSVGVITYM LLSGLSPFLG ETDAETMNFI VNCSWDFDAD TFEGLSEEAK |
| | DFVSRLLVKE KSCRMSATQC LKHEWLNNLP AKASRSKTRL KSQLLLQKYI AQRKWKKHFY |
| | VVTAANRLRK FPTSP |
| FN1 | MLRGPGPGLLLLAVQCLGTAVPSTGASKSKRQAQQMVQPQSPVAVSQSKPGCYDNGKHYQINQQ |
| | WERTYLGNALVCTCYGGSRGFNCESKPEAEETCFDKYTGNTYRVGDTYERPKDSMIWDCTCIGA |
| | GRGRISCTIANRCHEGGQSYKIGDTWRRPHETGGYMLECVCLGNGKGEWTCKPIAEKCFDHAAGT |
| | SYVVGETWEKPYQGWMMVDCTCLGEGSGRITCTSRNRCNDQDTRTSYRIGDTWSKKDNRGNLL |
| | QCICTGNGRGEWKCERHTSVQTTSSGSGPFTDVRAAVYQPQPHPQPPPYGHCVTDSGVVYSVGM |
| | QWLKTQGNKQMLCTCLGNGVSCQETAVTQTYGGNSNGEPCVLPFTYNGRTFYSCTTEGRQDGHL |
| | WCSTTSNYEQDQKYSFCTDHTVLVQTRGGNSNGALCHFPFLYNNHNYTDCTSEGRRDNMKWCG |
| | TTQNYDADQKFGFCPMAAHEEICTTNEGVMYRIGDQWDKQHDMGHMMRCTCVGNGRGEWTCI |
| | AYSQLRDQCIVDDITYNVNDTFHKRHEEGHMLNCTCFGQGRGRWKCDPVDQCQDSETGTFYQIG |
| | DSWEKYVHGVRYQCYCYGRGIGEWHCQPLQTYPSSSGPVEVFITETPSQPNSHPIQWNAPQPSHIS |
| | KYILRWRPKNSVGRWKEATIPGHLNSYTIKGLKPGVVYEGQLISIQQYGHQEVTRFDFTTTSTSTPV |
| | TSNTVTGETTPFSPLVATSESVTEITASSFVVSWVSASDTVSGFRVEYELSEEGDEPQYLDLPSTATS |
| | VNIPDLLPGRKYIVNVYQISEDGEQSLILSTSQTTAPDAPPDTTVDQVDDTSIVVRWSRPQAPITGYR |
| | IVYSPSVEGSSTELNLPETANSVTLSDLQPGVQYNITIYAVEENQESTPVVIQQETTGTPRSDTVPSP |
| | RDLQFVEVTDVKVTIMWTPPESAVTGYRVDVIPVNLPGEHGQRLPISRNTFAEVTGLSPGVTYYFK |
| | VFAVSHGRESKPLTAQQTTKLDAPTNLQFVNETDSTVLVRWTPPRAQITGYRLTVGLTRRGQPRQ |
| | YNVGPSVSKYPLRNLQPASEYTVSLVAIKGNQESPKATGVFTTLQPGSSIPPYNTEVTETTIVITWTP |
| | APRIGFKLGVRPSQGGEAPREVTSDSGSIVVSGLTPGVEYVYTIQVLRDGQERDAPIVNKVVTPLSP |
| | PTNLHLEANPDTGVLTVSWERSTTPDITGYRITTTPTNGQQGNSLEEVVHADQSSCTFDNLSPGLEY |
| | NVSVYTVKDDKESVPISDTIIPEVPQLTDLSFVDITDSSIGLRWTPLNSSTIIGYRITVVAAGEGIPIFE |
| | DFVDSSVGYYTVTGLEPGIDYDISVITLINGGESAPTTLTQQTAVPPPTDLRFTNIGPDTMRVTWAP |
| | PPSIDLTNFLVRYSPVKNEEDVAELSISPSDNAVVLTNLLPGTEYVVSVSSVYEQHESTPLRGRQKT |
| | GLDSPTGIDFSDITANSFTVHWIAPRATITGYRIRHHPEHFSGRPREDRVPHSRNSITLTNLTPGTEYV |
| | VSIVALNGREESPLLIGQQSTVSDVPRDLEVVAATPTSLLISWDAPAVTVRYYRITYGETGGNSPVQ |
| | EFTVPGSKSTATISGLKPGVDYTITVYAVTGRGDSPASSKPISINYRTEIDKPSQMQVTDVQDNSISV |
| | KWLPSSSPVTGYRVTTTPKNGPGPTKTKTAGPDQTEMTIEGLQPTVEYVVSVYAQNPSGESQPLV |
| | QTAVTNIDRPKGLAFTDVDVDSIKIAWESPQGQVSRYRVTYSSPEDGIHELFPAPDGEEDTAELQG |
| | LRPGSEYTVSVVALHDDMESQPLIGTQSTAIPAPTDLKFTQVTPTSLSAQWTPPNVQLTGYRVRVT |
| | PKEKTGPMKEINLAPDSSSVVVSGLMVATKYEVSVYALKDTLTSRPAQGVVTTLENVSPPRRARV |
| | TDATETTITISWRTKTETITGFQVDAVPANGQTPIQRTIKPDVRSYTITGLQPGTDYKIYLYTLNDNA |
| | RSSPVVIDASTAIDAPSNLRFLATTPNSLLVSWQPPRARITGYIIKYEKPGSPPREVVPRPRPGVTEAT |
| | ITGLEPGTEYTIYVIALKNNQKSEPLIGRKKTDELPQLVTLPHPNLHGPEILDVPSTVQKTPFVTHPG |
| | YDTGNGIQLPGTSGQQPSVGQQMIFEEHGFRRTTPPTTATPIRHRPRPYPPNVGEEIQIGHIPREDVD |
| | YHLYPHGPGLNPNASTGQEALSQTTISWAPFQDTSEYIISCHPVGTDEEPLQFRVPGTSTSATLTGLT |
| | RGATYNVIVEALKDQQRHKVREEVVTVGNSVNEGLNQPTDDSCFDPYTVSHYAVGDEWERMSES |
| | GFKLLCQCLGFGSGHFRCDSSRWCHDNGVNYKIGEKWDRQGENGQMMSCTCLGNGKGEFKCDP |
| | HEATCYDDGKTYHVGEQWQKEYLGAICSCTCFGGQRGWRCDNCRRPGGEPSPEGTTGQSYNQYS |
| | QRYHQRTNTNVNCPIECFMPLDVQADREDSRE |
| Col1a1 | MFSFVDLRLLLLLAATALLTHGQEEGQVEGQDEDIPPITCVQNGLRYHDRDVWKPEPCRICVCDN |
| | GKVLCDDVICDETKNCPGAEVPEGECCPVCPDGSESPTDQETTGVEGPKGDTGPRGPRGPAGPPGR |
| | DGIPGQPGLPGPPGPPGPPGPPGLGGNFAPQLSYGYDEKSTGGISVPGPMGPSGPRGLPGPPGAPGP |
| | QGFQGPPGEPGEPGASGPMGPRGPPGPPGKNGDDGEAGKPGRPGERGPPGPQGARGLPGTAGLPG |
| | MKGHRGFSGLDGAKGDAGPAGPKGEPGSPGENGAPGQMGPRGLPGERGRPGAPGPAGARGNDG |
| | ATGAAGPPGPTGPAGPPGFPGAVGAKGEAGPQGPRGSEGPQGVRGEPGPPGPAGAAGPAGNPGA |
| | DGQPGAKGANGAPGIAGAPGFPGARGPSGPQGPGGPPGPKGNSGEPGAPGSKGDTGAKGEPGPVG |
| | VQGPPGPAGEEGKRGARGEPGPTGLPGPPGERGGPGSRGFPGADGVAGPKGPAGERGSPGPAGPK |
| | GSPGEAGRPGEAGLPGAKGLTGSPGSPGPDGKTGPPGPAGQDGRPGPPGPPGARGQAGVMGFPGP |
| | KGAAGEPGKAGERGVPGPPGAVGPAGKDGEAGAQGPPGPAGPAGERGEQGPAGSPGFQGLPGPA |
| | GPPGEAGKPGEQGVPGDLGAPGPSGARGERGFPGERGVQGPPGPAGPRGANGAPGNDGAKGDAG |
| | APGAPGSQGAPGLQGMPGERGAAGLPGPKGDRGDAGPKGADGSPGKDGVRGLTGPIGPPGPAGA |
| | PGDKGESGPSGPAGPTGARGAPGDRGEPGPPGPAGFAGPPGADGQPGAKGEPGDAGAKGDAGPP |
| | GPAGPAGPPGPIGNVGAPGAKGARGSAGPPGATGFPGAAGRVGPPGPSGNAGPPGPPGPAGKEGG |
| | KGPRGETGPAGRPGEVGPPGPPGPAGEKGSPGADGPAGAPGTPGPQGIAGQRGVVGLPGQRGERG |
| | FPGLPGPSGEPGKQGPSGASGERGPPGPMGPPGLAGPPGESGREGAPGAEGSPGRDGSPGAKGDRG |
| | ETGPAGPPGAPGAPGAPGPVGPAGKSGDRGETGPAGPAGPVGPVGARGPAGPQGPRGDKGETGE |
| | QGDRGIKGHRGFSGLQGPPGPPGSPGEQGPSGASGPAGPRGPPGSAGAPGKDGLNGLPGPIGPPGP |
| | RGRTGDAGPVGPPGPPGPPGPPGPPSAGFDFSFLPQPPQEKAHDGGRYYRADDANVVRDRDLEVD |
| | TTLKSLSQQIENIRSPEGSRKNPARTCRDLKMCHSDWKSGEYWIDPNQGCNLDAIKVFCNMETGE |
| | TCVYPTQPSVAQKNWYISKNPKDKRHVWFGESMTDGFQFEYGGQGSDPADVAIQLTFLRLMSTE |
| | ASQNITYHCKNSVAYMDQQTGNLKKALLLQGSNEIEIRAEGNSRFTYSVTVDGCTSHTGAWGKTV |
| | IEYKTTKTSRLPIIDVAPLDVGAPDQEFGFDVGPVCFL |
| TGFB1 | MPPSGLRLLPLLLPLLWLLVLTPGRPAAGLSTCKTIDMELVKRKRIEAIRGQILSKLRLASPPSQGEV |
| | PPGPLPEAVLALYNSTRDRVAGESAEPEPEPEADYYAKEVTRVLMVETHNEIYDKFKQSTHSIYMF |
| | FNTSELREAVPEPVLLSRAELRLLRLKLKVEQHVELYQKYSNNSWRYLSNRLLAPSDSPEWLSFDV |
| | TGVVRQWLSRGGEIEGFRLSAHCSCDSRDNTLQVDINGFTTGRRGDLATIHGMNRPFLLLMATPLE |
| | RAQHLQSSRHRRALDTNYCFSSTEKNCCVRQLYIDFRKDLGWKWIHEPKGYHANFCLGPCPYIWS |
| | LDTQYSKVLALYNQHNPGASAAPCCVPQALEPLPIVYYVGRKPKVEQLSNMIVRSCKCS |

### REFERENCES

Vulto et al. Lab Chip, 2011,11, 1596-1602
Mondrinos et al. (2021) Science Advances, doi: 10.1126/sciadv.abe9446
1 Huh, D. et al. Microfabrication of human organs-on-chips. Nat Protoc 8, 2135-2157, doi:10.1038/nprot.2013.137 (2013).
2 Naderi, A., Bhattacharjee, N. & Folch, A. Digital Manufacturing for Microfluidics. Annu Rev Biomed Eng 21, 325-364, doi:10.1146/annurev-bioeng-092618-020341 (2019).
3 Au, A. K., Lee, W. & Folch, A. Mail-order microfluidics: evaluation of stereolithography for the production of microfluidic devices. Lab Chip 14, 1294-1301, doi:10.1039/c31c51360b (2014).
4 Novak, R. et al. Scalable Fabrication of Stretchable, Dual Channel, Microfluidic Organ Chips. J Vis Exp, doi:10.3791/58151 (2018).
5 de Almeida Monteiro Melo Ferraz, M., Nagashima, J. B., Venzac, B., Le Gac, S. & Songsasen, N. 3D printed mold leachates in PDMS microfluidic devices. Sci Rep 10, 994, doi:10.1038/s41598-020-57816-y (2020).
6 Kress, S. et al. 3D Printing of Cell Culture Devices: Assessment and Prevention of the Cytotoxicity of Photopolymers for Stereolithography. Materials (Basel) 13, doi:10.3390/ma13133011 (2020).
7 Villegas, M., Cetinic, Z., Shakeri, A. & Didar, T. F. Fabricating smooth PDMS microfluidic channels from low-resolution 3D printed molds using an omniphobic lubricant-infused coating. Anal Chim Acta 1000, 248-255, doi:10.1016/j.aca.2017.11.063 (2018).
8 Huh, D. et al. Reconstituting organ-level lung functions on a chip. Science 328, 1662-1668, doi:10.1126/science.1188302 (2010).
9 Polacheck, W. J., Charest, J. L. & Kamm, R. D. Interstitial flow influences direction of tumor cell migration through competing mechanisms. Proc Natl Acad Sci USA 108, 11115-11120, doi:10.1073/pnas.1103581108 (2011).
10 Vulto, P. et al. Phaseguides: a paradigm shift in microfluidic priming and emptying. Lab Chip 11, 1596-1602, doi:10.1039/c01c00643b (2011).
11 Trietsch, S. J. et al. Membrane-free culture and real-time barrier integrity assessment of perfused intestinal epithelium tubes. Nat Commun 8, 262, doi:10.1038/s41467-017-00259-3 (2017).
12 Wang, X. et al. Engineering anastomosis between living capillary networks and endothelial cell-lined microfluidic channels. Lab Chip 16, 282-290, doi:10.1039/c5lc01050k (2016).
13 Zervantonakis, I. K. et al. Three-dimensional microfluidic model for tumor cell intravasation and endothelial barrier function. Proc Natl Acad Sci USA 109, 13515-13520, doi:10.1073/pnas.1210182109 (2012).
14 van Duinen, V. et al. Perfused 3D angiogenic sprouting in a high-throughput in vitro platform. Angiogenesis 22, 157-165, doi:10.1007/s10456-018-9647-0 (2019).
Mondrinos, M. J. et al. Surface-directed engineering of tissue anisotropy in microphysiological models of musculoskeletal tissue. Sci Adv 7, doi:10.1126/sciadv.abe9446 (2021).
16 Wang, Y. I., Carmona, C., Hickman, J. J. & Shuler, M. L. Multiorgan Microphysiological Systems for Drug Development: Strategies, Advances, and Challenges. Adv Healthc Mater 7, doi:10.1002/adhm.201701000 (2018).
17 McAleer, C. W. et al. Multi-organ system for the evaluation of efficacy and off-target toxicity of anticancer therapeutics. Sci Transl Med 11, doi:10.1126/scitranslmed.aav1386 (2019).
18 Sung, J. H. et al. Recent Advances in Body-on-a-Chip Systems. Anal Chem 91, 330-351, doi:10.1021/acs.analchem.8b05293 (2019).
19 Sung, J. H., Wang, Y. & Shuler, M. L. Strategies for using mathematical modeling approaches to design and interpret multi-organ microphysiological systems (MPS). APL Bioeng 3, 021501, doi:10.1063/1.5097675 (2019).
Liu, Y. & Cao, X. Characteristics and Significance of the Pre-metastatic Niche. Cancer Cell 30, 668-681, doi:10.1016/j.ccell.2016.09.011 (2016).
21 Malhab, L. J. B. et al. Chronic Inflammation and Cancer: The Role of Endothelial Dysfunction and Vascular Inflammation. Curr Pharm Des 27, 2156-2169, doi:10.2174/1381612827666210303143442 (2021).
22 Mondrinos, M. J. et al. Pulmonary endothelial protein kinase C-delta (PKCdelta) regulates neutrophil migration in acute lung inflammation. Am J Pathol 184, 200-213, doi:10.1016/j.ajpath.2013.09.010 (2014).
23 Mondrinos, M. J., Yi, Y. S., Wu, N. K., Ding, X. & Huh, D. Native extracellular matrix-derived semipermeable, optically transparent, and inexpensive membrane inserts for microfluidic cell culture. Lab Chip, doi:10.1039/c71c00317j (2017).
24 Park, S. E., Georgescu, A., Oh, J. M., Kwon, K. W. & Huh, D. Polydopamine-Based Interfacial Engineering of Extracellular Matrix Hydrogels for the Construction and Long-Term Maintenance of Living Three-Dimensional Tissues. ACS Appl Mater Interfaces 11, 23919-23925, doi:10.1021/acsami.9b07912 (2019).
25 Ngo, P., Ramalingam, P., Phillips, J. A. & Furuta, G. T. Collagen gel contraction assay. Methods Mol Biol 341, 103-109, doi:10.1385/1-59745-113-4:103 (2006).
26 Venzac, B. et al. PDMS Curing Inhibition on 3D-Printed Molds: Why? Also, How to Avoid It? Anal Chem 93, 7180-7187, doi:10.1021/acs.analchem.0c04944 (2021).
27 McDonald, J. C. et al. Fabrication of microfluidic systems in poly(dimethylsiloxane). Electrophoresis 21, 27-40, doi:10.1002/(SICI)1522-2683(20000101)21:1<27::AID-ELPS27>3.0.CO;2-C (2000).
28 Duffy, D. C., McDonald, J. C., Schueller, O. J. & Whitesides, G. M. Rapid Prototyping of Microfluidic Systems in Poly(dimethylsiloxane). Anal Chem 70, 4974-4984, doi:10.1021/ac980656z (1998).
29 Sun, L. et al. Tailoring Materials with Specific Wettability in Biomedical Engineering. Adv Sci (Weinh) 8, e2100126, doi:10.1002/advs.202100126 (2021).
30 Cortese, B. et al. Superhydrophobicity due to the hierarchical scale roughness of PDMS surfaces. Langmuir 24, 2712-2718, doi:10.1021/la702764x (2008).
31 Kim, S., Chung, M., Ahn, J., Lee, S. & Jeon, N. L. Interstitial flow regulates the angiogenic response and phenotype of endothelial cells in a 3D culture model. Lab Chip 16, 4189-4199, doi:10.1039/c6lc00910g (2016).
32 de Matos-Neto, E. M. et al. Systemic Inflammation in Cachexia—Is Tumor Cytokine Expression Profile the Culprit? Front Immunol 6, 629, doi:10.3389/fimmu.2015.00629 (2015).
33 Mondrinos, M. J., Kennedy, P. A., Lyons, M., Deutschman, C. S. & Kilpatrick, L. E. Protein kinase C and acute respiratory distress syndrome. Shock 39, 467-479, doi:10.1097/SHK.0b013e318294f85a (2013).
34 Moya, M. L., Hsu, Y. H., Lee, A. P., Hughes, C. C. & George, S. C. In vitro perfused human capillary networks. Tissue Eng Part C Methods 19, 730-737, doi:10.1089/ten.TEC.2012.0430 (2013).
35 Polacheck, W. J., Kutys, M. L., Tefft, J. B. & Chen, C. S. Microfabricated blood vessels for modeling the vascular transport barrier. Nat Protoc 14, 1425-1454, doi:10.1038/s41596-019-0144-8 (2019).
36 Polacheck, W. J. et al. A non-canonical Notch complex regulates adherens junctions and vascular barrier function. Nature 552, 258-262, doi:10.1038/nature24998 (2017).
37 Ansari, A., Trehan, R., Watson, C. & Senyo, S. Increasing Silicone Mold Longevity: A Review of Surface Modification Techniques for PDMS-PDMS Double Casting. Soft Mater 19, 388-399, doi:10.1080/1539445x.2020.1850476 (2021).
38 Qin, D., Xia, Y. & Whitesides, G. M. Soft lithography for micro- and nanoscale patterning. Nat Protoc 5, 491-502, doi:10.1038/nprot.2009.234 (2010).
39 Wu, H., Huang, B. & Zare, R. N. Construction of microfluidic chips using polydimethylsiloxane for adhesive bonding. Lab Chip 5, 1393-1398, doi:10.1039/b510494g (2005).
40 Mondrinos, M. J., Jones, P. L., Finck, C. M. & Lelkes, P. I. Engineering de novo assembly of fetal pulmonary organoids. Tissue Eng Part A 20, 2892-2907, doi:10.1089/ten.TEA.2014.0085 (2014).
41 Holthofer, H. et al. Ulex europaeus I lectin as a marker for vascular endothelium in human tissues. Lab Invest 47, 60-66 (1982).
42 Zhang, K., Zuo, W., Chen, Y., Meng, D. & Zhang, L. Beyond a Gaussian Denoiser: Residual Learning of Deep CNN for Image Denoising. IEEE Trans Image Process 26, 3142-3155, doi:10.1109/TIP.2017.2662206 (2017).
43 Corliss, B. A. et al. REAVER: A program for improved analysis of high-resolution vascular network images. Microcirculation 27, e12618, doi:10.1111/micc.12618 (2020).

1. A composition comprising:
(a) a solid substrate comprising a first compartment and a second compartment; the first and second compartments in fluid communication with each other;
(b) a plurality of cancer cells;
(c) a plurality of endothelial cells; and
wherein the cancer cells and the endothelial cells are in separate compartments in fluid communication; and
wherein the composition is membrane-free.

2. The composition of claim 1 wherein the composition is not more than 2 millimeters in length.

3. The composition of claim 1, wherein the endothelial cells are structurally organized in an in vitro blood vessel structure fluidly connecting the cancer cells with a group of non-cancer cells.

4. The composition of claim 1, wherein a layer of hydrogel is positioned at the interface of the first and second compartments.

5. (canceled)

6. The composition of claim 1, wherein the hydrogel has a density above about 75 mg/per mL of hydrogel volume.

7.-8. (canceled)

9. The composition of claim 1, wherein the cancer cells are from a biopsy sample is triple negative breast cancer.

10.-11. (canceled)

12. The composition of any of claims 1 through 8, wherein the cancer cells are carcinoma cells from a human lung, human breast or human colon.

13.-16. (canceled)

17. The composition of claim 1, wherein one or a plurality of compartments has a volume of from about 30 to about 50 millimeter cubed.

18. The composition of claim 1, further comprising stromal cells in a layer of hydrogel.

19.-20. (canceled)

21. The composition of claim 1, wherein the stromal cells are at density from about 500,000 cells per milliliter of volume of the compartment to about 1,500,000 cells per milliliter of volume of the compartment.

22.-24. (canceled)

25. A method of assaying the toxicity or therapeutic effectiveness of an agent on a cancer cell comprising:
a. contacting the composition of claim 1 with an agent.

26. The method of claim 28 further comprising a step of (b) monitoring the cells for morphologic changes or changes of expression profile of cells after step (a).

27. The method of claim 25, wherein the agent is chosen from one or a combination of: an environmental agent, a small molecule therapeutic, a biologic immunotherapy, or a modified T cell.

28. The method of claim 27, wherein the agent is a biologic immunotherapy that is an antibody or antibody fragment thereof.

29. The method of claim 27, wherein the agent is a modified cell that is a CAR-T cell.

30. (canceled)

31. A method of manufacturing a cell culture comprising:
(a) seeding a plurality of cancer cells; and
(b) seeding a plurality of endothelial cells for a time period sufficient for the endothelial cells to fluidically connect the first and second compartment of the composition of claim 1.

32. The method of claim 31, wherein the time period is no less than about seven days.

33. The method of claim 31, wherein the composition further comprises a layer of stromal cells and extracellular matrix protein or proteins positioned at the interface of the first and second compartment, and wherein the time period is sufficient to deposit extracellular protein density around the plurality of cancer cells equivalent to from about 6 KPa to about 10 KPa.

34. The method of claim 33 further comprising allowing the cells to divide until there are from about 400,000 cells per milliliter to about 1,000,000 of stromal cells per milliliter of volume in a vessel.

35. The method of claim 31, wherein the composition is capable of fluid exchange through diffusion between the first or second compartments.
